(19) United States
(12) Patent Application Publication
(10) Pub. No.: US 2022/0229065 A1
(43) Pub. Date: Jul. 21, 2022

(54) SENSORS AND METHODS FOR RAPID MICROBIAL DETECTION

(71) Applicant: BAMBU VAULT LLC, Lowell, MA (US)

(72) Inventors: Prakash RAI, Lowell, MA (US); Satish C. AGRAWAL, Sudbury, MA (US); Michael P. FILOSA, Medfield, MA (US)

(73) Assignee: BAMBU VAULT LLC, Lowell, MA (US)

(21) Appl. No.: 17/613,424

(22) PCT Filed: May 22, 2020
(86) PCT No.: PCT/US2020/034390

§ 371 Date: Nov. 22, 2021

Related U.S. Application Data
(60) Provisional application No. 62/960,793, filed on Jan. 14, 2020.
(60) Provisional application No. 62/852,694, filed on May 24, 2019.
(60) Provisional application No. 62/852,698, filed on May 24, 2019.

(51) Int. Cl. G01N 33/58

(52) U.S. Cl. CPC G01N 33/582; G01N 2333/986; G01N 33/581

(57) ABSTRACT

The disclosure provides biosensors, diagnostic compositions, diagnostic particles, theranostic particles thereof and methods of use thereof to detect drug resistant microbes and destroy them using an exogenous source.

### CROSS-REFERENCE TO RELATED APPLICATIONS

The present application claims the benefit of priority to U.S. Provisional Application No. 62/852,694, filed May 24, 2019, U.S. Provisional Application No. 62/852,698, filed May 24, 2019, and U.S. Provisional Application No. 62/960,793, filed, Jan. 14, 2020, each of which are incorporated herein by reference in its entirety for all purposes.

### FIELD OF INVENTION

The disclosure provides biosensors, diagnostic particles, theragnostic particles thereof and methods of use thereof to rapidly detect drug resistant microbes and destroy them using an exogenous source. Rationally designed libraries are devised for the detection of microbes using screening tools that are well established in the field.

### BACKGROUND OF THE INVENTION

Microbial infection can cause serious complications during wound healing after surgery. It is important to accelerate tissue regeneration in order to minimize the possibility of bacterial infection. Conventional treatments for microbial infection are antibiotics (e.g., penicillin or cephalosporin) which are becoming less efficient owing to the emergence of antibiotic-resistant bacterial strains. Rather than using routine antibiotic therapies, a better approach is to practice good wound management, e.g. keep the area free from bacteria before, during and after surgery, and carefully monitor the wound site for infection during healing.

Unfortunately, due to widespread use (and even abuse) of broad-spectrum antibiotics, microbes, especially bacteria, have evolved to develop multiple ways to resist treatments. Drug-resistant microbial infections are increasingly becoming a major healthcare problem worldwide, especially in the hospital setting with surgical site infections (SSI). The Centers for Disease Control and Prevention (CDC) released a report in November 2019 that underscores the threat of antibiotic resistance in the U.S., where more than 2.8 million antibiotic-resistant infections occur each year and more than 35,000 people die as a result. The CDC estimates that there were more than 323,000 methicillin resistant Staphylococcus aureus (MRSA) cases in hospitalized patients in the U.S. and more than 10,000 deaths in 2017. Some reports suggest that the actual numbers may be higher than these CDC estimates. The growing incidence of MRSA infections are adding to the expanding healthcare costs. Current ways to diagnose resistant SSI's such as MRSA can take up to 48-72 h and during this time, the patient is put on conventional antibiotics that can have severe side effects. Recently the FDA approved a test that can diagnose MRSA in 5 hours, but it needs specialized equipment. A swift diagnosis of drug resistant microbes is thus crucial for appropriate treatment and improves the success of the treatment of HAI.

The existing technique of swabbing the infected wound and culturing is costly and time-consuming. This delay between swabbing and obtaining a positive or negative result undoubtedly causes untimely or unnecessary treatment. Therefore, there exists a need for new methods to rapidly detect and destroy any microbial infection before it spreads from a specific site into the blood.

The ability to detect deadly drug-resistant microbial infections in minutes instead of the several hours it currently takes will dramatically improve the management of hospital-acquired infections (HAI) and community-acquired infections (CAI). In July 2019, six babies and six adults (hospital staff) at the University of Pittsburgh Medical Center Children's Hospital NICU, were confirmed to have a MRSA infection. It was reported that some of the staff were showing symptoms while the infants were not, suggesting the staff had the infection before it was transmitted to the infant patients. A MRSA infection outbreak in the hospital setting can be deadly given the presence of immuno-comprised patients and/or infants who haven't developed an immune system. After an MRSA outbreak, affected patients are isolated and medical staff wear protective gear until it is safe. Usually the isolation will last for as long as the patient is in the hospital which adds to the healthcare costs. Rapid detection and disinfection of equipment, instruments, hospital beds, railings, and all the other hospital associated areas would help reduce the spread of MRSA and the associated costs. Novel molecules can be rationally designed for rapid detection of microbes. Using rationally designed libraries for screening against microbes can yield such molecules with excellent sensitivity and specificity.

For decades, hospitals have worked to get doctors, nurses and other health care workers to wash their hands and prevent the spread of germs. However, a new study (https://academic.oup.com/cid/article/69/11/1837/5445425) suggests they should expand those efforts to their patients, too. In the study, 14 percent of 399 hospital patients tested had “superbug” antibiotic-resistant bacteria on their hands or nostrils very early in their hospital stay, the research finds. And nearly a third of tests for such bacteria on objects that patients commonly touch in their rooms, such as the nurse call button, came back positive. An additional 6 percent of the patients who didn't have multidrug-resistant organisms, or MDROs, on their hands at the start of their hospitalization tested positive for them on their hands later in their stay. One-fifth of the objects tested in their rooms had similar superbugs on them, too. The research team of this study cautions that the presence of MDROs on patients or objects in their rooms does not necessarily mean that patients will get sick with antibiotic-resistant bacteria. Further, they note that health care workers' hands are still the primary mode of microbe transmission to patients. Within the hospital, healthcare workers (HCWs) are often exposed to infections. Any transmissible disease can occur in the hospital setting and may affect HCWs. HCWs are not only at risk of acquiring infections but also of being a source of infection to patients. Therefore, both the patient and the HCW need to be protected from contracting or transmitting hospital-acquired infections. A quick and inexpensive way to identify hospital beds, equipment's instruments, health care workers, patients and/or even visitors that may be carrying MDRO's like MRSA would be highly beneficial in stopping the spread of these HAI or CAI. Approximately 75% of community acquired MRSA infections are localized to the skin and soft tissues and can generally be effectively treated, if detected early. However, these strains exhibit enhanced virulence, spread more rapidly, cause more serious diseases, affect life support organs, and may cause widespread infection (sepsis), toxic shock syndrome, and pneumonia.

Therefore, there is a need for early and rapid detection of microbes like MRSA. The present invention provides a solution to meet such need.

### SUMMARY OF THE INVENTION

In an embodiment, this disclosure provides a biosensor for the detection of drug resistant bacteria comprising a first material FG responsive to an antibacterial inactivating factor secreted by the drug resistant bacteria; and a spectroscopic probe D, wherein FG is coupled to the spectroscopic probe, wherein FG masks the activity of D, wherein the antibacterial inactivating factor causes FG to decouple from D, resulting in a detectable optical response.

In some embodiments, the spectroscopic probe D is selected from the group consisting of a fluorophore, a chromophore, an infrared chromophore, a visible light chromophore, and combinations thereof.

### BRIEF DESCRIPTION OF THE DRAWINGS

FIG. 1 is a flowchart of the feedback loop for identifying optimal diagnostic particle structure guided by ECT/EDP.

FIG. 2 is a flowchart of the feedback loop for identifying optimal theranostic particle structure guided by ECT/EDP/TCT.

FIG. 3 illustrates the particle size distribution measured by Horiba LA-950 particle size analyzer in de-ionized water with pH 7.4.

FIG. 4 illustrates the degradation of Epolight™ 1117 measured at 1064 nm wavelength after exposure to 80° C.

FIG. 5 illustrates schematic transwell plate for TCT and cross-section showing the two cell types.

FIG. 6 illustrates the controlled heat generation from laser excited Epolight™ 1117 IR dye-loaded particles dispersed in gelatin. A red 50° C. thermochromic dye was suspended in gelatin as an indicator of heat generation by the color change from red color to colorless. Spots 1, 4, 5, 6, 7 of FIG. 6 were exposed to laser irradiation from a Lutronic laser with a pulse width of 10 ns operated under Q-switched mode. Spots 2 and 3 were exposed with the Lutronic laser with a pulse width of 350 [is. Spots 8-16 were exposed with a semiconductor laser using various pulse widths from 10-250 ms.

FIG. 7 illustrates the suspension of red thermochromic dye prior to laser exposure.

FIG. 8 illustrates the color change at spot 9 after two exposures with a semiconductor laser operated at a wavelength of 980 nm with a pulse width of 250 ms to produce a total fluence of 70.7 J/cm2.

FIGS. 9A, 9B and 9C illustrate the melting of gelatin and decolorization of red dye without any clearing of the IR dye at the spots 15 and 16 after laser irradiation at 980 nm and a total fluence of 14.7 J/cm2 (FIG. 9B, Spot 15) and 14.1 J/cm2 (FIG. 9C, Spot 16).

### DETAILED DESCRIPTION OF THE INVENTION

The pace of diagnostic processes in clinical microbiology laboratories has largely been unchanged for almost 100 years, as availability of diagnostic results essentially depended on the growth of bacteria. Using traditional approaches, it takes at least 24 hours for obtaining growth from clinical specimens, and an additional 24 hours for down-stream isolate characterization (i.e., biochemical identification and phenotypic susceptibility testing). Consequently, therapeutic decisions are commonly made empirically until the availability of species identification and resistance patterns.

The emergence of pathogens carrying acquired resistance determinants, e.g, methicillin-resistant Staphylococcus aureus (MRSA), extended spectrum β-lactamase-(ESBL) producing Enterobacteriaceae, or carbapenem-resistant Gram-negative rods, has resulted in increasingly broad empiric treatment regimens, often including glycopeptides and broad-spectrum β-lactams such as piperacillin-tazobactam or carbapenems. The resulting overuse of these reserved agents itself drives the emergence and spread of multi-resistant organisms. The situation is aggravated by the often unsuccessful recovery of pathogens from patients receiving prior broad-spectrum antibiotics and, in consequence, unavailability of subsequent drug susceptibility data. Moreover, it is a common problem that the successful empiric broad-spectrum therapy remains in place although microbiological test results justify de-escalation. Therefore, it is evident that overtreatment is, at least partially, linked to the discrepancy between traditional microbiological procedures and the clinical need for more rapid results.

### Definitions

As used in the preceding sections and throughout the rest of this specification, unless defined otherwise, all the technical and scientific terms used herein have the same meaning as is commonly understood by one of skill in the art to which this invention belongs. All patents and publications referred to herein are incorporated by reference in their entireties.

Amino acids are represented in three-letter code or one letter code, as illustrated in the table below.

| Amino acid | Three letter code | One letter code |
|---|---|---|
| alanine | Ala | A |
| arginine | Arg | R |
| asparagine | Asn | N |
| aspartic acid | Asp | D |
| asparagine or aspartic acid | Asx | B |
| cysteine | Cys | C |
| glutamic acid | Glu | E |
| glutamine | Gln | Q |
| glutamine or glutamic acid | Glx | Z |
| glycine | Gly | G |
| histidine | His | H |
| isoleucine | Ile | I |
| leucine | Leu | L |
| lysine | Lys | K |
| methionine | Met | M |
| phenylalanine | Phe | F |
| proline | Pro | P |
| serine | Ser | S |
| threonine | Thr | T |
| tryptophan | Trp | W |
| tyrosine | Tyr | Y |
| valine | Val | V |

As used, herein the term “alkyl” refers to a straight or branched hydrocarbon chain radical consisting solely of carbon and hydrogen atoms, containing no unsaturation, having from one to ten carbon atoms (e.g., (C₁₋₁₀)alkyl or C₁₋₁₀ alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Examples of “alkyl” as used herein include, but are not limited to, methyl, ethyl, propyl, isopropyl, isobutyl, n-butyl, tert-butyl, isopentyl, and n-pentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —N(R^{a})₂, where each R^{a} is independently hydrogen, fluoroalkyl, carbocyclyl, carbocyclylalkyl, aryl, aralkyl, heterocycloalkyl, heterocycloalkylalkyl, heteroaryl or heteroarylalkyl.

As used herein, the term “aryl” refers to a benzene ring or to a fused benzene ring system, for example anthracene, phenanthrene, or naphthalene ring systems. Examples of “aryl” groups include, but are not limited to, phenyl, biphenyl, naphthyl, indenyl, azulenyl, fluorenyl, anthracenyl, phenanthrenyl, tetrahydronaphthyl, indanyl, phenanthridinyl and the like. Unless otherwise indicated, the term “aryl” also includes each possible positional isomer of an aromatic hydrocarbon radical, such as in 1-naphthyl, 2-naphthyl, 5-tetrahydronaphthyl, 6-tetrahydronaphthyl, 1-phenanthridinyl, 2-phenanthridinyl, 3-phenanthridinyl, 4-phenanthridinyl, 7-phenanthridinyl, 8-phenanthridinyl, 9-phenanthridinyl and 10-phenanthridinyl and the like. One preferred aryl group is phenyl.

As used herein the term “halogen” refers to fluorine, chlorine, bromine, or iodine.

As used herein the term “haloalkyl” refers to an alkyl group, as defined herein that is substituted with at least one halogen. Examples of branched or straight chained “haloalkyl” groups useful in the present invention include, but are not limited to, methyl, ethyl, propyl, isopropyl, n-butyl, and t-butyl substituted independently with one or more halogens, e.g., fluoro, chloro, bromo, and iodo. The term “haloalkyl” should be interpreted to include such substituents such as —CF₃, —CH₂—CH₂—F, —CH₂—CF₃, and the like.

As used herein, the term “oxo” refers to a group ═O.

As used herein the term “alkoxy” refers to a group —ORₐ, where Rₐ is alkyl as herein defined.

As used herein the term “cyano” refers to a group —CN.

As used herein throughout the present specification, the phrase “optionally substituted” or variations thereof denote an optional substitution, including multiple degrees of substitution, with one or more substituent group, preferably one or two. The phrase should not be interpreted to be imprecise or duplicative of substitution patterns herein described or depicted specifically.

Esters of the compounds of the present invention are independently selected from the groups consisting of (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted by, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters, and (5) mono-, di- or triphosphate esters. The phosphate esters may be further esterified by, for example, a C₁₋₂₀ alcohol or reactive derivative thereof, or by a 2, 3-di (C₆₋₂₄)acyl glycerol.

In such esters, unless otherwise specified, any alkyl moiety present advantageously contains from 1 to 18 carbon atoms, particularly from 1 to 6 carbon atoms, more particularly from 1 to 4 carbon atoms. Any cycloalkyl moiety present in such esters advantageously contains from 3 to 6 carbon atoms. Any aryl moiety present in such esters advantageously comprises a phenyl group.

Ethers of the compounds of the present invention include, but are not limited to methyl, ethyl, butyl and the like.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocyclyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C₂₋₁₀)alkenyl or C₂₋₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Carboxaldehyde” refers to a —(C═O)H radical.

“Carboxyl” refers to a —(C═O)OH radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. (C₃₋₁₀)cycloalkyl or C₃₋₁₀ cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{d}, and NR^{d}R^{d} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon), and heteroalicyclic (bonded through a ring carbon), each of which moiety may itself be optionally substituted. The R₂ of —N(R)₂ of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety.

The terms “a,” “an,” and “the” as used herein, generally are construed to cover both the singular and the plural forms.

The term “about” as used herein, generally refers to a particular numeric value is within an acceptable error range as determined by one of ordinary skill in the art, which will depend in part on how the numeric value is measured or determined, i.e., the limitations of the measurement system. For example, “about” can mean a range of ±20%, ±10%, or ±5% of a given numeric value.

The term “absorption” of energy as used herein, generally refers to the process of matter taking up exogenous energy that transforms the state of that matter to a higher electronic state when interacting with an exogenous source described herein. The process of absorption leads to an attenuation in the intensity of the energy of the exogenous source.

The term “body chemicals” as used herein, generally refers to the existing chemicals in any one of the bodily fluids, neutrophil media, macrophage media, or any complete cell growth media.

The term “bodily fluid” as used herein, generally refers to the natural fluid found in one of the fluid compartments of the human body. The principal fluid compartments are intracellular and extracellular. A much smaller segment, the transcellular compartment, includes fluid in the tracheobronchial tree, the gastrointestinal tract, and the bladder; cerebrospinal fluid; and the aqueous humor of the eye. The bodily fluid includes blood plasma, serum, cerebrospinal fluid, or saliva. In an embodiment, the bodily fluid contains neutrophils and macrophages.

The terms “antimicrobial inactivating factor” as used herein refers to an enzyme secreted by the microbes that degrades the antibiotic, thereby inactivating it. An existing cellular enzyme is modified to react with the antibiotic in such a way that it no longer affects the microorganism. For example, the penicillinases are a group of β-lactamase enzymes that cleave the β-lactam ring of the penicillin molecule. Alternatively, a specific enzyme modifies the antibiotic in a way that it loses its activity, e.g., streptomycin; the antibiotic is chemically modified so that it will no longer bind to the ribosome to block protein synthesis. Some of the well-characterized “antimicrobial inactivating factors” include β-lactamase, erythromycin (macrolide) esterase, and amphenicolhydrolase. The FG is the material interacting with the antimicrobial inactivating factor.

As used herein, “β-lactam” refers to an antibiotic containing a β-lactam ring in its molecular structure. As is well known to those skilled in the art, β-lactams encompass for example derivatives of penicillin, cephalosporins, monobactams and carbapenemes.

The term “biocompatibility” as used herein, refers to the capability of a material implanted in the body to perform with an appropriate host response in a specific application without causing deleterious changes.

The term “biocompatible polymer” as used herein, generally refers to polymers that are intended to interface with biological systems to evaluate, treat, augment or replace any tissue, organ or function of the body. Some of the characteristic properties of the biocompatible polymers include “not having toxic or injurious effects on biological systems,” “the ability of a polymer to perform with an appropriate host response in a specific application,” and “ability of a polymer to perform its desired function with respect to a medical therapy, without eliciting any undesirable local or systemic effects in the recipient or beneficiary of that therapy, but generating an appropriate beneficial cellular or tissue response in that specific situation, and optimizing the clinically relevant performance of that therapy.”

As used herein, the term “chromophore” refers to a molecule or a part of a molecule responsible for its color. Color arises when a molecule absorbs certain wavelengths of visible light and transmits or reflects others. A molecule having an energy difference between two different molecular orbitals falling within the range of the visible spectrum may absorb visible light and thus be aptly characterized as a chromophore. Visible light incident on a chromophore may be absorbed thus exciting an electron from a ground state molecular orbital into an excited state molecular orbital.

“Chitosan” refers to a cationic polysaccharide derived from chitin, a biopolymer found in the shells of crustaceans. Generally, chitosan is obtained by removing about 50% or more of acetyl groups of C2 acetamide from chitin, and chitosan generally has a degree of acetylation of less than 50%. Chitosan comprises (1,4)-linked N-acetyl-D-glucosamine and D-glucosamine units. Chitosan exhibits relatively poor water solubility.

As used herein, the term “click chemistry” refers to copper (I)-catalyzed alkyne-azide cycloaddition (CuAAC), or strain-promoted alkyne-azide cycloaddition (SPAAC), or thiol-yne click reactions.

“Degree of acetylation” refers to the ratio or percentage of amine groups along the backbone of a chitosan or chitosan derivative molecule (such as glycol chitosan or glycol chitin) that are acetylated.

The term “EDC-NHS” refers to chemical reactions to form amide bonds. First, 1-ethyl-3-(3-dimethylaminopropyl) carbodiimide hydrochloride (EDC) reacts with a molecule containing a carboxylic-acid group, forming an amine-reactive O-acyl isourea intermediate. This intermediate may further react with N-hydroxysuccinimide (NHS) or N-hydroxysulfosuccinimide (sulfo-NHS) to form a semi-stable amine-reactive NHS ester, which further reacts with a compound containing an amine, yielding a conjugate of the two molecules (the carboxylic acid and the amine) joined by a stable amide bond.

The term “Efficacy Determination Protocol” for the biosensor in the diagnostic particle as used herein, generally refers to the method used for determining the degree of the degradation of the biosensor inside a diagnostic particle, wherein the biosensor interacts with body chemicals. After being treated with body chemicals for a period of time that simulates the use environment, the stability of the biosensor is evaluated by measuring the amount of the biosensor preserved. Various analytical tools, like UV-VIS-NIR, NMR, HPLC, LCMS, etc., would be used to quantify the concentration of the biosensor in the extracts and control. The details of the Efficacy Determination Protocol are described in Example section of the disclosure. In some instances, if the degradation of the biosensor is less than 90% after being subject to the body chemical, then the diagnostic particle is considered passing the Efficacy Determination Protocol.

The term “Efficacy Determination Protocol” for the second material in the theragnostic particle as used herein, generally refers to the method used for determining the degree of the degradation of the second material inside a theragnostic particle, wherein the second material interacts with body chemicals. After being treated with body chemicals for a period of time that simulates the use environment, the stability of the second material is evaluated by measuring the amount of the second material preserved. Various analytical tools, like UV-VIS-NIR, NMR, HPLC, LCMS, etc., would be used to quantify the concentration of the second material in the extracts and control. The details of the Efficacy Determination Protocol are described in Example section of the disclosure. In some instances, if the degradation of the second material is less than 90% after being subject to the body chemical, then the theragnostic particle is considered passing the Efficacy Determination Protocol.

The term “energy-to-heat conversion efficiency” describes the percentage of absorbed exogenous energy that is converted into heat, as determined by a rise in temperature.

The term “Extractable Cytotoxicity Test” as used herein, generally refers to an in vitro leaching protocol (using physiologically relevant media that contains serum proteins at physiological temperature) can be used to extract the material from the particles. The extract can then be used as is (“neat” or 1×) or in serial dilutions (up to 10,000× dilutions) with the media in a cytotoxicity test against healthy cells (different cells will be chosen depending upon the application) as a surrogate measurement for the porosity of the particles. The neat or dilution of the extract that kills 30% of the cells can be measured and is referred to as an IC₃₀. Likewise, the neat or dilution of the extract that kills 10% of the cells can be measured and is referred to as an IC₁₀. The neat or dilution of the extract that kills 20% of the cells or below can be measured and is referred to as an IC₂₀. The neat or dilution of the extract that kills 40% or below of the cells can be measured and is referred to as an IC₄₀. The neat or dilution of the extract that kills 50% or below of the cells can be measured and is referred to as an IC₅₀. The neat or dilution of the extract that kills 60% or below of the cells can be measured and is referred to as an IC₆₀. The neat or dilution of the extract that kills 70% or below of the cells can be measured and is referred to as an IC₇₀. The neat or dilution of the extract that kills 80% or below of the cells can be measured and is referred to as an IC₈₀. The neat or dilution of the extract that kills 90% or below of the cells can be measured and is referred to as an IC₉₀. Details of the Extractable Cytotoxicity Test are described in Examples section of the disclosure. The Extractable Cytotoxicity Test is compliant with the international standards: ISO-10993-5 “Tests for cytotoxicity-in vitro methods”. In some instances, if the neat or dilution concentration of the material in the leachate is independently less than IC₁₀, IC₃₀, IC₄₀, IC₅₀, IC₆₀, IC₇₀, IC₈₀, or IC₉₀, the particle passes the Extractable Cytotoxicity Test.

The term “electromagnetic radiation” (EMR) as used herein, generally refers to a complex system of radiant energy composed of waves and energy bundles that are organized according to the length of the propagating wave. It includes radio waves, microwaves, infrared (IR) radiation, visible light, LED light, ultraviolet light, X-rays, and gamma rays.

The term “energy fluence” as used herein, generally refers to the areal density of the energy contained within the light and expressed in the units of energy per unit area, for example, joules/m² or joules/cm².

As used herein, the term “esterase” refers to an enzyme from the group of hydrolases splitting esters into an acid and an alcohol. Esterases differ according to their substrate specificity, their protein structure and their biological function. Esterases particularly comprise triphosphoric monoester hydrolases, sulfatases, diphosphoric monoester hydrolases, phosphoric triester hydrolases, exodeoxyribonucleases, exoribonucleases, exonucleases, deoxyribonucleases, ribonucleases, endodeoxyribonucleases and endoribonucleases.

The term “feedback loop” as used herein, generally refers to a feedback loop based on the Extractable Cytotoxicity Test and/or Efficacy Determination Protocol, which have been utilized to evaluate if a particle needs to be rendered less porous by altering the chemistry of the particle fabrication. In the Extractable Cytotoxicity Test, when cell death is less than 30% then the particles are considered to have passed the Extractable Cytotoxicity Test. The Extractable Cytotoxicity Test is compliant with the international standards: ISO-10993-5 “Tests for cytotoxicity-in vitro methods.”

As used herein, the term “fluorophore” refers to a group of atoms within a molecule that is responsible for the capability of this molecule to emit fluorescence light after excitation with the appropriate wavelengths of light. They are generally substances composed of several conjugated aromatic rings, or they are planar or cyclic molecules having one or more it bonds.

The term “hydrogel” as used herein refers to a three-dimensional network made of cross-linked hydrophilic or amphiphilic polymers that are swollen in liquid without dissolving in them. Hydrogels have the capability to absorb a large amount of water. Hydrogels are low-volume-fraction 3D networks of molecules, fibers or particles with intermediate voids, filled with water or aqueous media. Hydrogels can be classified into two classes: one class is physical gel resulting from physical association of polymer chains, and the other class is chemical gels (or irreversible gel) in which the network is linked by covalent bonds. The inclusion of functional groups as pendant groups or on the backbone of the 3D network allows the synthesis of hydrogels that swell in response to a variety of stimuli including temperature, electromagnetic fields, chemicals and biomolecules.

The term “hydrophilic,” as used herein, refers to the property of having affinity for water. For example, hydrophilic polymers (or hydrophilic polymer segments) are polymers (or polymer segments) which are primarily soluble in aqueous solutions and/or have a tendency to absorb water. In general, the more hydrophilic a polymer is, the more that polymer tends to dissolve in, mix with, or be wetted by water. Generally, materials with a water contact angle of less than 90° are considered to be hydrophilic.

The term “hydrophobic,” as used herein, refers to the property of lacking affinity for, or even repelling water. For example, the more hydrophobic a polymer (or polymer segment), the more that polymer (or polymer segment) tends to not dissolve in, not mix with, or not be wetted by water. Generally, materials with a water contact angle of greater than 90° are considered to be hydrophobic.

The term “infrared radiation” or “infrared” (IR) as used herein, generally refers to electromagnetic radiation (EMR) with longer wavelengths than those of visible light. IR wavelengths extend from the nominal red edge of the visible spectrum at 700 nm (frequency 430 THz), to 1 mm (300 GHz). A wide range of substances absorbs IR, causing them to increase in temperature as the vibrations dissipate as heat.

The term “localized surface plasmon resonance” (LSPRs, localized SPRs) as used herein refers to collective electron charge oscillations in metallic nanoparticles that are excited by light. In contrast with the case of bulk metal, when light having various wavelengths is emitted onto a material existing on a local surface such as metal nanoparticles, polarization occurs on the surface of metal nanoparticles and exhibits a unique characteristic of increasing the intensity of the electric field. Electrons formed by polarization form a group (plasmon) and locally vibrate on the surface of the metal nanoparticles. This phenomenon is called localized surface plasmon resonance (LSPR). They exhibit enhanced near-field amplitude at the resonance wavelength.

The term “a material” as used herein, including “the first material” and “the second material”, refers to the material that interacts with an exogenous source described in the disclosure.

The term “Material Process Stability” as used herein refers to the preservation of the optical and physical characteristics of the second material under conditions of use such that it can deliver heat as intended upon stimulation by the exogenous source.

The term “near infrared radiation” (NIR) as used herein, generally refers to commonly used subdivision scheme for Infrared EMR with wavelengths extending from 750 nm (400 THz) to 1400 nm (214 THz).

The term “Nd:YAG” as used herein, generally refers to Neodymium-doped Yttrium Aluminum Garnet (YAG) a widely used solid-state crystal composed of yttrium and aluminum oxides and a small amount of the rare earth neodymium.

The term “photothermal conversion efficiency” describes the percentage of absorbed radiant energy that is converted into heat, as determined by a rise in temperature.

The term “photothermal therapy” (PTT) as used herein refers to a minimally invasive therapy in which photonic energy is converted into heat in order to kill cells such as cancer cells, microbes, virus, and bacteria.

The term “Polydispersity Index (PdI)” is defined as the square of the ratio of standard deviation (σ) of the particle diameter distribution divided by the mean particle diameter (2a), as illustrated by the formula: PdI=(σ/2a)². PdI is used to estimate the degree of non-uniformity of a size distribution of particles, and larger PdI values correspond to a larger size distribution in the particle sample. PdI can also indicate particle aggregation along with the consistency and efficiency of particle surface modifications. A sample is considered monodisperse when the PdI value is less than 0.1.

The term “power” as used herein, generally refers to the rate at which energy is emitted from a laser and is expressed in watts or milliwatts.

The term “power density (irradiance)” as used herein, generally refers to the quotient of incident laser power on a unit surface area, expressed as watts/cm² (W/cm²).

The term “pulse” as used herein, generally refers to the brief span of time for which, the focused and scanned laser beam interacts with a given point on the skin (usually ranging from picoseconds to milliseconds).

The term “Q-Switch” as used herein, generally refers to an optical device (e.g., Pockels cell) that controls the storage or release of laser energy from a laser optical cavity. Q-switching is a means of creating very short pulses (5-100 ns) with extremely high peak powers. Q stands for quality.

The term “synergistic,” or “synergistic effect” or “synergism” as used herein, generally refers to an effect such that the one or more effects of the combination of compositions is greater than the one or more effects of each component alone, or they can be greater than the sum of the one or more effects of each component alone. The synergistic effect can be greater than a percent value selected from the group consisting of about 10%, 20%, 30%, 40%, 50%, 60%, 75%, 100%, 110%, 120%, 150%, 200%, 250%, 350%, and 500% than the effect on a subject with one of the components alone, or the additive effects of each of the components when administered individually. The effect can be any of the measurable effects described herein. Advantageously, such synergy between the agents when combined, may allow for the use of smaller doses of one or both agents, may provide greater efficacy at the same doses, and may prevent or delay the build-up of multi-drug resistance. The combination index (CI) method of Chou and Talalay may be used to determine the synergy, additive or antagonism effect of the agents used in combination. When the CI value is less than 1, there is synergy between the compounds used in the combination; when the CI value is equal to 1, there is an additive effect between the compounds used in the combination and when CI value is more than 1, there is an antagonistic effect. The synergistic effect may be attained by co-formulating the agents of the pharmaceutical combination. The synergistic effect may be attained by administering two or more agents as separate formulations or in one particle, administered simultaneously or sequentially.

The term “therapeutic index” (TI) as used herein refers to a quantitative measurement of the relative safety of a drug. It is a comparison of the amount of a therapeutic agent that causes the therapeutic effect to the amount that causes toxicity.

$$TI = \frac{T D 50}{E D 50} ,$$

where ED₅₀ is median effective dose and TD₅₀ is the median toxic dose. The median effective dose (ED₅₀) is the dose at which 50% of the subjects exhibit the required effect of the drug. The median toxic dose (TD₅₀) is the dose required to produce a defined toxic effect in 50% of subjects. For many drugs, there are severe toxicities that occur at sublethal doses in humans, and these toxicities often limit the maximum dose of a drug. A high therapeutic index (TI) is preferable for a drug to have a favorable safety and efficacy profile.

The term “thermal cytotoxicity test” as used herein refers to an in vitro test specifically designed to test the compositions and the specific exogenous source(s) for their ability to kill the microbial cells while sparing the healthy host cells. The thermal cytotoxicity test is a trans-well assay wherein two different cells types, one being the microbial cells with the other type being the healthy (host) cells, are grown in the same well and exposed to different doses of the composition and the exogenous source. Viabilities of the two cells types are assessed a day after exposure of the cells to the compositions and exogenous source using standard colorimetric assays. Different types of microbial or normal (host) cells can be selected for this test for different applications. The composition and light dose(s) that do not kill any more than 30% of the healthy (host) cells but kill at least 70% of the microbial cells are considered passing the thermal cytotoxicity test.

The term “thermal relaxation time (TRT)” as used herein, generally refers to a simplified mathematical model to estimate the time taken for the target to dissipate about 50% of the incident thermal energy. It is related to the size of the targeted particle, e.g., 10 picoseconds (˜4 nm particle), 400 picoseconds (˜50 nm particle), a few nanoseconds (particles ranging in size from 40-300 nm), 200-1000 nanoseconds (melanosomes, ˜0.5 μm), to hundreds of milliseconds (e.g., leg venules). Longer TRT means the target takes longer time to cool to 50% of the temperature achieved. For spherical targets with radius R the TRT may be determined using Eqn. (I). TRT=R²/6.75 k, Eqn. (I) where k is thermal diffusivity. For R=10 nanometers, 50 nanometers, and 5 nanometers, TRT is about 160 picoseconds, 4 nanoseconds, and 40 picoseconds, respectively. Even if the epidermis is a strong competing absorber, it can be spared as long as the TRT of the target is longer than that of epidermis (3-5 milliseconds).

As used herein, the term “visible light” refers to a portion of the electromagnetic spectrum that is visible to the human eye. A typical human eye will respond to wavelengths from about 380 to 740 nanometers. The spectrum does not contain all the colors that the human eyes and brain can distinguish. Unsaturated colors such as pink, or purple variations like magenta, for example, are absent because they can only be made from a mix of multiple wavelengths. Colors containing only one wavelength are also called pure colors or spectral colors. Colors that can be produced by visible light of a narrow band of wavelengths (monochromatic light) are called pure spectral colors. The spectral various color include violet (380-450 nm), blue (450-485 nm), cyan (485-500 nm), green (500-565 nm), yellow (565-590 nm), orange (590-625 nm) and red (625-740 nm).

### Biosensors for Rapid Detection of Drug Resistant Microbes.

In an embodiment, this disclosure provides a biosensor for the rapid detection of drug resistant bacteria comprising a first material FG responsive to an antibacterial inactivating factor secreted by the drug resistant bacteria; and a spectroscopic probe D, wherein FG is coupled to the spectroscopic probe, wherein FG masks the activity of D, wherein the antibacterial inactivating factor causes FG to decouple from D, resulting in a detectable optical response.

In an embodiment, the biosensor described herein has a Formula (1) D-FG, wherein: (i) FG comprises a material responsive to an antimicrobial inactivating factor secreted by a microbes; and (ii) D is a spectroscopic probe, wherein FG is conjugated to the spectroscopic probe D via a covalent bond.

In some embodiments, the spectroscopic probe D is selected from the group consisting of a fluorophore, a chromophore, an infrared chromophore, a visible light chromophore, and combinations thereof.

### The Fragmentable Group (FG)

Many drug-resistant bacteria have developed biochemical mechanisms to deactivate antibiotics used to combat them. These mechanisms include enzymes that can break down the chemical structures that are essential to the action of the antibiotic, usually by hydrolysis.

In some embodiments, the antimicrobial inactivating factors include enzymes secreted by the microbes that are selected from the group consisting of β-lactamase, erythromycin (macrolide) esterase, chloramphenicol (phenicol) hydrolase, and combinations thereof. In some embodiments, the enzymes secreted by the microbe is β-lactamase. In some embodiments, the β-lactamase is selected from the group consisting of penicillinases, cephalosporinases, carbenicillinases, oxacillinases, carbapenemases including the metallo-β-lactamases, extended spectrum β-lactamases (ESBL), and combinations thereof. In some embodiments, the enzymes secreted by the microbe is erythromycin esterase. In some embodiments, the enzymes secreted by the microbe is chloramphenicol (phenicol) hydrolase.

The hydrolytic inactivation of antibiotics has been demonstrated for many antimicrobial agents, among which the β-lactam resistance mediated by β-lactamase is the most well-known. β-lactamases are responsible for resistance to β-lactams such as penicillins, cephalosporins, monobactams, cephamycins, and carbapenems. In addition, the macrolide esterase and an amphenicol hydrolase were also shown to mediate the related drug resistance.

β-Lactamases are bacterial enzymes that inactivate β-lactam containing antibiotics by hydrolyzing the β-lactam rings, such as opening the amide bond of the β-lactam ring. β-lactamases are widely distributed in bacterial organisms but are not found in mammalian cells. β-lactamases are found in gram positive and gram-negative bacteria. β-lactamases are numerous and diversified. β-lactamases are divided into two main classifications: (1) the Bush classification determined in 1989, and updated in 1995 and again in 2009, classifies β-lactamases in relation to their preferred substrate from among penicillin, oxacillin, carbenicillin, cefaloridine, cefotaxime and imipenem, and in relation to their susceptibility to clavulanic acid, a β-lactamase inhibitor; (2) the Ambler classification proposed in 1980 is based on the protein sequence of β-lactamases. β-lactamases are classified into four molecular classes A to D. Classes A, C and D have a serine residue at their active site and class B, or metallo-β-lactamases, have zinc at their active site. According to the Bush classification, β-lactamases conferring multidrug resistance include carbapenemases, AmpC cephalosporinases, and extended-spectrum β-lactamases (ESBL). Other antibiotic inactivating factors include erythromycin esterases and amphenicol hydrolase.

AmpC cephalosporinases are β-lactamases that confer resistance to cephalosporin antibiotics (including cephamycins) in Enterobacter, Nitrobacteria, Morganella, Serratia, and P. aeruginosa. AmpC β-lactamases are class C enzymes. AmpC β-lactamases hydrolyze broad and extended-spectrum cephalosporins (i.e., cephamycins (CMY) and oxyimino-β lactams, e.g. CMY-family AmpC). AmpC or cephalosporinases exhibits a greater hydrolysis for cephalosporins in comparison to benzylpenicillin. These enzymes are inhibited or partially inhibited by class A β-lactamase inhibitors such as clavulanate or tazobactam.

Extended-spectrum β-lactamases (ESBLs) are β-lactamases that hydrolyze cephalosporins with an oxyimino chain. ESBL hydrolyzes late-generation cephalosporins (such as cefotaxime (CTX-M)). ESBL causes resistance to most β-lactam antibiotics with the exceptions of the cephamycins (cefoxitin, cefotetan) and carbapenems. Extended-spectrum β-lactamases (ESBL) cause resistance to most β-lactam antibiotics with the exceptions of the cephamycins (cefoxitin, cefotetan) and carbapenems. The most common bacteria carrying ESBL are Klebsiella spp. and Escherichia coli. Less commonly Enterobacter, Serratia, Morganella, Proteus, and Pseudomonas aeruginosa spp. may harbor these genes. ESBL-producing bacteria are also often resistant to aminoglycosides and quinolones. These enzymes are usually inhibited by β-lactamase inhibitors such as clavulanic acid, sulbactam, and tazobactam. ESBL-producing bacteria are also often resistant to aminoglycosides and quinolones. ESBLs include the TEM family, SHV family as well as others, and CTX-M family, which are class A β-lactamases.

Carbapenemases are enzymes that can inactivate the carbapenems (meropenem, imipenem-cilastatin, ertapenem, and doripenem). Carbapenemases are a diverse group of β-β-lactamases that include enzymes belonging to class A, B and D. Class A carbapenemases include KPC-1, KPC-2, KPC-3 and KPC-4. Class B carbapenemases include the IMP family, VIM family, GIM-1 and SPM-1 as well as others. Class D carbapenemases include OXA-23, OXA-24, OXA-25, OXA-26, and OXA-27, OXA-40 and OXA-40 as well as others. These enzymes may also be able to inactivate all classes of β-lactam antibiotics and are resistant to β-lactamase inhibitors. Organisms found to carry these MDR genes include P. aeruginosa, Acinetobacter, Stenotrophomonas, Klebsiella, Serratia, Enterobacter, E. coli, and Citrobacter. In 2009, a new carbapenemase was isolated in pathogens from New Delhi, India, called the New Delhi metallo-β-lactamase-1 (NDM-1). Bacteria harboring the NDM-1 include Klebsiella, E. coli, Enterobacter, Nitrobacteria, Morganella, Providencia, Acinetobacter, and P. aeruginosa.

In some embodiments, the FG comprises a fragment derived from a β-lactam antibiotic, macrolide antibiotic, or amphenicol antibiotic.

In some embodiments, the FG is derived from a β-lactam antibiotic. In some embodiments, the β-lactam antibiotic is selected from the group consisting of benzylpenicillin, phenoxymethylpenicillin, propicillin, pheneticillin, azidocillin, clometocillin, penamecillin, cloxacillin, dicloxacillin, flucloxacillin, oxacillin, nafcillin, methicillin, amoxicillin, ampicillin, pivampicillin, hetacillin, bacampicillin, metampicillin, talampicillin, epicillin, ticarcillin carbenicillin, carindacillin, temocillin, piperacillin, azlocillin, mezlocillin, mecillinam, pivmecillinam, sulbenicillin, faropenem, ritipenem, ertapenem, antipseudomonal, doripenem, imipenem, meropenem, biapenem, panipenem, cephalothin (also known as cefalotin), cefazolin, cefazaflur, cefalexin, cefadroxil, cefapirin, cefazedone, cefazaflur, cefradin, cefroxadin, ceftezole, cefaloglycin, cefacetril, cefalonium, cefaloridin, cefalotin, cefalonium, cefapirin, cefatrizine, cefazedon, cefaclor, cefotetan, cephamycin, cefoxitin, cefprozil, cefuroxime, axetil, cefamandole, cefminox, cefonicid, ceforanide, cefotiam, cefbuperazone, cefuzonam, cefmetazole, carbacephem, loracarbef, cefixime, ceftriaxone, antipseudomonal, ceftazidime, cefoperazone, cefdinir, cefcapene, cefdaloxime, ceftizoxime, cefmenoxime, cefotaxime, cefpiramide, cefpodoxime, ceftibuten, cefditoren, cefetamet, cefodizime, cefpimizole, cefsulodin, cefteram, ceftiolene, oxacephem, flomoxef, latamoxef, cefozopran, cefpirome, cefquinome, ceftaroline, fosamil, ceftolozane, ceftobiprole, ceftiofur, cefquinome, and cefovecin.

In some embodiments, the FG is derived from the cephalosporin class of antibiotics

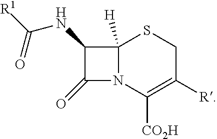

In some embodiments, the cephalosporin is selected from the group consisting of cephalothin (also known as cefalotin), cefazolin, cefazaflur, cefalexin, cefadroxil, cefapirin, cefazedone, cefazaflur, cefradin, cefroxadin, ceftezole, cefaloglycin, cefacetril, cefalonium, cefaloridin, cefalotin, cefalonium, cefapirin, cefatrizine, cefazedon, cefaclor, cefotetan, cephamycin, cefoxitin, cefprozil, cefuroxime, cefamandole, cefminox, cefonicid, ceforanide, cefotiam, cefbuperazone, cefuzonam, cefmetazole, cefixime, ceftriaxone, ceftazidime, cefoperazone, cefdinir, cefcapene, cefdaloxime, ceftizoxime, cefmenoxime, cefotaxime, cefpiramide, cefpodoxime, ceftibuten, cefditoren, cefetamet, cefodizime, cefpimizole, cefsulodin, cefteram, ceftiolene, cefozopran, cefpirome, cefquinome, ceftaroline, ceftolozane, ceftobiprole, ceftiofur, cefquinome, and cefovecin.

Cephalosporins are usually classified into cephalosporins of first, second, third or fourth generation based on their spectrum of activity and their greater or lesser resistance or stability against β-lactamases. This classification is well known to persons skilled in the art (Barber et al. (2004) Adv Biochem Eng Biotechnol. 88:179-215).

In some embodiments, the FG is derived from the first generation cephalosporins selected from the group consisting of cephazolin, cephalothin, cephapirin, cephaloridin, cephalexin, cephradine, and cefadroxil. In some embodiments, the FG is derived from the first generation cephalosporin cephalothin

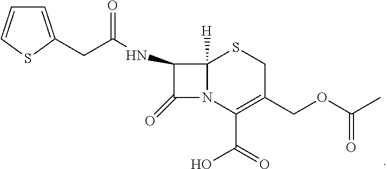 [SMILES: CC(=O)OCC1=C(C(=O)O)N2C(=O)[C@@H](NC(=O)Cc3cccs3)[C@H]2SC1]

In some embodiments, the FG is derived from a second generation cephalosporin selected from the group consisting of cefamandole, cefuroxime, cefonicide, ceforanide, cefatrizine, cefotiam, cefprozil, loracarbef, cefotixin, and cefaclor.

In some embodiments, the FG is derived from the third generation cephalosporins selected from the group consisting of cefcapene, cefcapene pivoxil, cefdaloxime, cefdaloxime civoxil, cefdinir, cefditoren, cefixime, cefinenoxime, cefodizime, cefoperazone cefotaxime s-oxide, cefotaxime, benzathine cefotaxime, desacetylcefotaxime, cefpimizole, cefpiramide cefpodoxime, ceftazidime, cefteram, cefteram pivaloyloxymethyl ester, ceftibuten, trans-ceftibuten, ceftiofur, desfuroylceftiofur, ceftiolene, ceftizoxime, ceftriaxone, and the salts thereof.

In some embodiments, the FG is derived from the third generation cephalosporins selected from the group consisting of ceftriaxone, cefotaxime, ceftazidime, cefixime, cefpodoxime, cefsulodin, cefatamet, cefoperazone, ceftibuten,), and the salts thereof.

In some embodiments, the FG is derived from the fourth generation cephalosporins selected from the group consisting of cefepime and cefpirome.

In some embodiments, the FG is derived from the β-lactam antibiotic deactivated by cephalosporinase and is selected from the group consisting of penicillins, cephalothin, first generation cephalosporins, and some 2nd generation and third generation cephalosporins.

In some embodiments, the FG is derived from the β-lactam antibiotic deactivated by penicillinase specifically and is selected from the group consisting of penicillins and cephalothin.

In some embodiments, the FG is derived from the β-lactam antibiotic deactivated by carbenicillinase and is selected from the group consisting of cephalothin, carbenicillins, penicillins, and cloxacillins.

In some embodiments, the FG is derived from the β-lactam antibiotic deactivated by oxacillinase and is selected from the group consisting of cephalothin, cloxacillins, penicillins and carbenicillins.

In some embodiments, the FG is derived from the β-lactam antibiotic deactivated by carbapenemases and is selected from the group consisting of cephalothin, penicillins, cephalosporins, and carbapenemes.

In some embodiments, the FG is derived from the β-lactam antibiotic deactivated by metallo-β-lactamase and is selected from the group consisting of cephalothin, penicillins, cephalosporins, and carbapenemes.

In some embodiments, the FG is derived from the β-lactam antibiotic deactivated by extended spectrum β-lactamase and is selected from the group consisting of cephalothin, penicillins, cephalosporins, and third generation cephalosporins.

In some embodiments, the FG comprises a fragment having

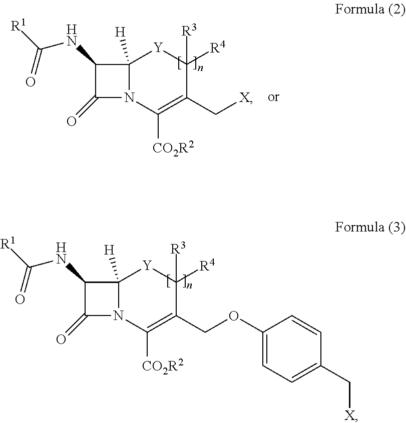

wherein R¹ is selected from the group consisting of —CH₂—CN, —CH₂—S—CH₂—CN, —CH₂—CF₃, —CH₂—CHF₂, —CH₂—O-Ph, —CH(-Me)(—O-Ph), —CH(-Et)(O-Ph), —CH₂-Ph,

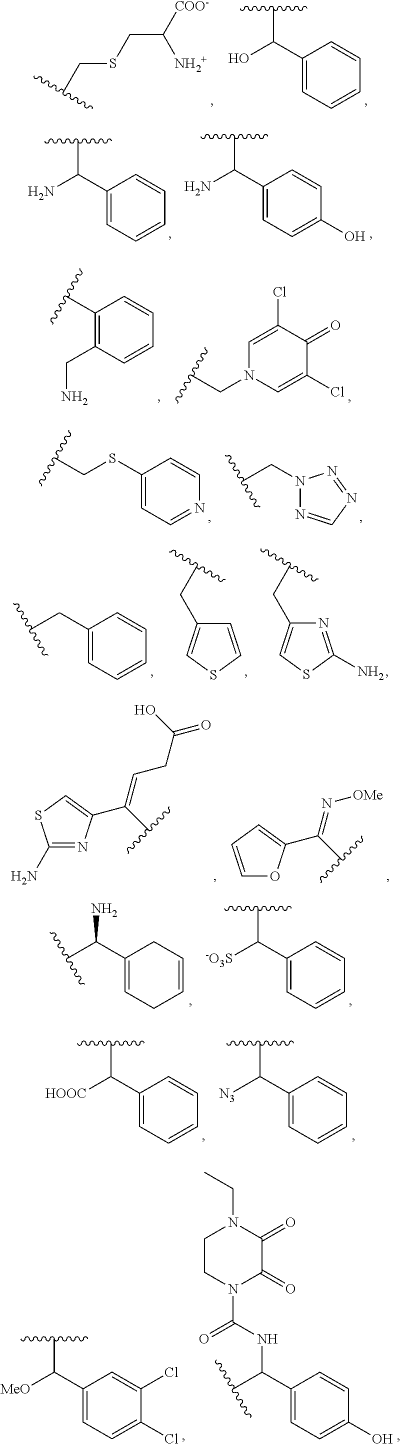

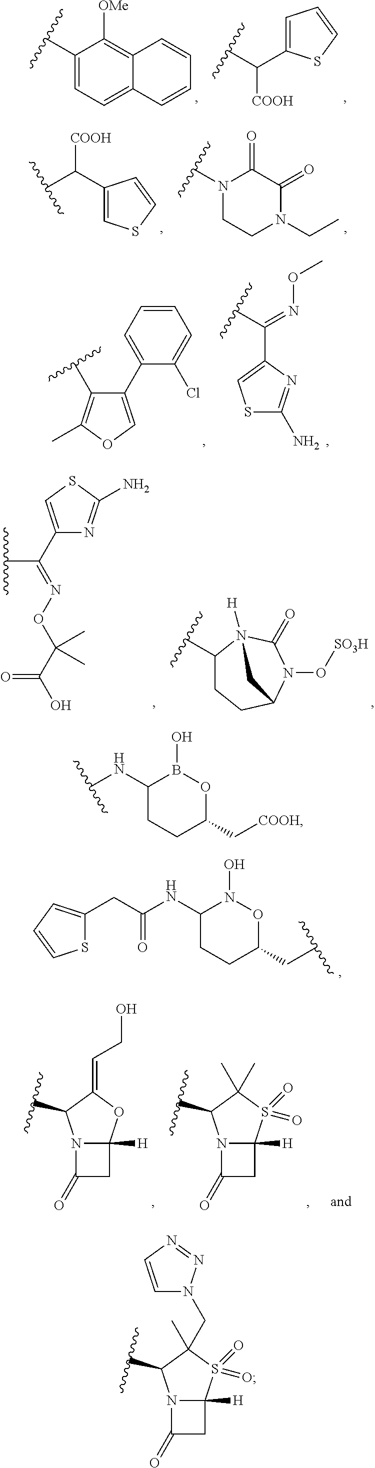

R², R³, and R⁴ are each independently selected from the group consisting of H, substituted and unsubstituted C₁-C₁₂ alkyl group, substituted and unsubstituted C₁-C₁₂ alkenyl group, substituted and unsubstituted C₁-C₁₂ alkynyl group, and substituted and unsubstituted aryl group;

Y is a bond, S, or O; and

X represents the point of attachment to the spectroscopic probe D.

Macrolide antibiotics such as azithromycin and erythromycin are mainstays of modern antibacterial chemotherapy, and like all antibiotics, they are vulnerable to resistance. One mechanism of macrolide resistance is via drug inactivation by enzymatic hydrolysis of the macrolactone ring catalyzed by erythromycin esterases, EreA and EreB.

In some embodiments, FG is derived from erythromycin. In some embodiments, the FG comprises a fragment having

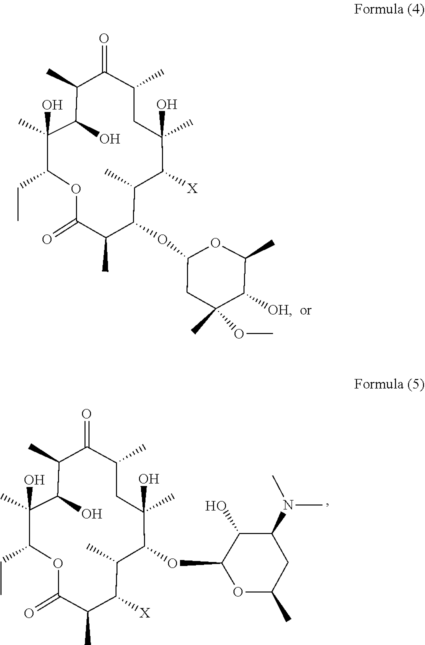

wherein X represents the point of attachment to the spectroscopic probe D.

In some embodiments, the FG is derived from an amphenicol antibiotic. Amphenicols are a class of antibiotics with a phenylpropanoid structure. They function by blocking the enzyme peptidyl transferase on the 50S ribosome subunit of bacteria. For example, chloramphenicol and florfenicol are broad-spectrum antibiotics. The inactivation of chloramphenicol by numerous bacteria has been detected repeatedly. Hydrolysis of chloramphenicol has been recognized in cell extracts of Escherichia coli expressing a chloramphenicol acetate esterase gene, EstDL136. When EstDL136 was expressed in E. coli, EstDL136 conferred resistance to both chloramphenicol and florfenicol on E. coli, due to their inactivation.

In some embodiments, the FG comprises a fragment having

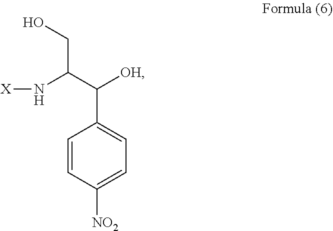

wherein X represents the point of attachment to the spectroscopic probe D.

This molecular design is not limited to beta-lactamase producing pathogens. It can be extended to pathogens like Candida which overproduce lipases or to pathogens that overexpress enzymes such as tyrosinase. Peptidases which cleave specific proteins can also be incorporated into this design to produce color specific to an overexpressed peptidase. In some embodiments, FG is derived from

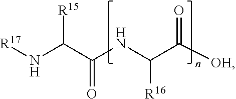

wherein the biosensor is sensitive to peptidase secreted by the drug resistant microbes.

In some embodiments, FG is derived from

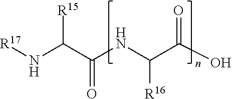

having a peptide sequence selected from the group consisting of Met-Leu-Ala-Arg-Arg-Lys-Pro-Val-Leu-Pro-Ala-Leu-Thr-Ile-Asn-Pro-Thr-Ile (Bacillus anthracis lethal factor); Tyr-Phe-Glu-Gly-Ser-Leu-Gly-Glu-Asp-Asp-Asn-Leu-Asp (Clostridium difficile A toxin)); Leu-Val-Leu-Gly-Ser-Ser-Leu-Val-Leu-Gly-Ser-Ser or Phe-Leu-Leu-Asp-Ala-Ala-Pro-Cys-Glu-Pro (Staphylococcus aureus staphopain A); Ile-Val-Phe-Gly-Gly-Ser-Ile-Val-Phe-Gly-Gly-Ser or Ile-Thr-Phe-Gly-Ala-Ser-Ile-Thr-Phe-Gly-Ala-Ser (Staphylococcus aureus staphopain B); Gly-Phe-Leu-Pro-Arg-His-Arg-Asp-Thr-Gly-Ile-Leu-Asp-Ser (Staphylococcus aureus V8); Gln-Gln-Thr-Gln-Ser-Ser-Lys-Gln-Gln-Thr-Pro-Lys-Ile-Gln (Staphylococcus aureus SspA); Trp-Leu-Tyr-Thr-Ser-Tyr-Leu-Tyr-Ser-Ser (Staphylococcus aureus Spls); Ser-His-Leu-Gly-Leu-Ala-Arg-Ser-Asn-Leu-Asp-Glu-Asp-Ile-Ile-Ala-Glu (Staphylococcus aureus Aureolysin); Val-Ser-Arg-Arg-Arg-Arg-Arg-Gly-Gly-Cys (E coli, OmpT); Ala-Ala-Ile-Lys-Ala-Gly-Ala-Arg-Asp-Lys-Val-Asn-Leu-Gly-Gly-Gln (Streptococcus pyogenes, SpeB); Ser-Ala-Ala-Ile-Lys-Ala-Gly-Ala (Streptococcus pyogenes, SpeC); Asn-X-Cys-Pro-Pro-Tyr-Pro-Cys (Streptococcus pneumonia, IgA specific serine endopeptidase); Leu-tyr-Leu-Tyr-Trp-Leu-Tyr-Leu-Tyr-Trp (Streptococcus pneumonia, ClpP); Val-Lys-Leu-Glu-Gln-Phe-Lys-Glu-Val-Thr-Glu (Streptococcus pneumonia, HtrA); Lys-Arg-Leu-Phe-Lys-Glu-Leu-Lys-Phe-Ser-Leu-Arg-Lys-Tyr (Enterococcus faecalis, SprE); Ser-Ala-Gln-Thr-Phe-Ser-Ala-Leu-Ser-Pro-Thr (Enterococcus faecalis, GelE), and combinations thereof, wherein the biosensor is sensitive to peptidase secreted by the drug resistant microbes.

In some embodiments, FG has a peptide sequence Met-Leu-Ala-Arg-Arg-Lys-Pro-Val-Leu-Pro-Ala-Leu-Thr-Ile-Asn-Pro-Thr-Ile (Bacillus anthracis lethal factor). In some embodiments, A has a peptide sequence Val-Ser-Arg-Arg-Arg-Arg-Arg-Gly-Gly-Cys (E coli, OmpT). In some embodiments, A has a peptide sequence Asn-X-Cys-Pro-Pro-Tyr-Pro-Cys (Streptococcus pneumonia, IgA specific serine endopeptidase). In some embodiments, FG has a peptide sequence Leu-tyr-Leu-Tyr-Trp-Leu-Tyr-Leu-Tyr-Trp (Streptococcus pneumonia, ClpP) or Val-Lys-Leu-Glu-Gln-Phe-Lys-Glu-Val-Thr-Glu (Streptococcus pneumonia, HtrA). In some embodiments, FG has a peptide sequence Gln-Gln-Thr-Gln-Ser-Ser-Lys-Gln-Gln-Thr-Pro-Lys-Ile-Gln (Staphylococcus aureus SspA) or Trp-Leu-Tyr-Thr-Ser-Tyr-Leu-Tyr-Ser-Ser (Staphylococcus aureus Spls).

In some embodiments, FG is

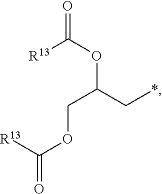

wherein R¹³ is a C12, C14, C16, or C18 linear alkyl or alkenyl group, and wherein the biosensor is effective for detecting Candida albicans.

In some embodiments, FG is

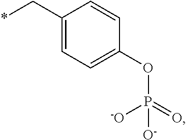

wherein the biosensor is effective for detecting microbes secreting phosphatase.

In some embodiments, FG is

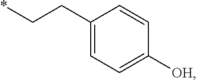 [SMILES: *CCc1ccc(O)cc1]

wherein the biosensor is effective for detecting microbes secreting tyrosinase.

In some embodiments, FG is

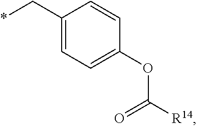

wherein the biosensor is effective for detecting microbes secreting esterase.

In some embodiments, FG is selected from the group consisting of

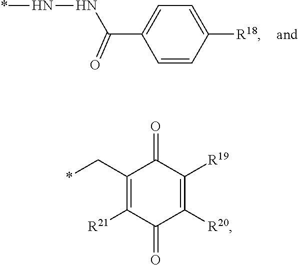

wherein the biosensor is sensitive to redox microenvironment surrounding microbes.

### Bimodal β-Lactamase Sensing Component

This disclosure provides a colorimetric β-lactamase biosensor. The design features of the disclosed colorimetric biosensor have two main components: (1) a chromogenic probe or fluorogenic probe, (2) a bimodal sensing component for β-lactamase having a first material derived from β-lactam antibiotic and a third material derived from β-lactamase inhibitor. β-lactamase degrades the first material to release the chromogenic probe or fluorogenic probe to produce detectable optical response (e.g. change of color, or emitting fluorescence). The third material in the biosensor acts to enhance the selectivity toward microbes that secrete specific type antibiotic inactivating factor (a targeted approach).

Among the β-lactamases, β-lactamase TEM-1 is most widespread in the different bacterial species. It hydrolyzes penicillins most efficiently, but not third generation cephalosporins and it is susceptible to inhibition by clavulanic acid. Penicillinases hydrolyzes generally only penicillins and sometimes early-generation cephalosporins. Bacteria producing β-lactamase TEM-1 and penicillinases can easily be detected with a first material derived from first or second-generation cephalosporins. Class A carbapenemases that hydrolyze penicillins, cephalosporins, and carbapenems (e.g. Klebsiella pneumonia carbapenemase KPC). These enzymes are inhibited or partially inhibited by class A inhibitors such as clavulanate or tazobactam. Thus, the selectivity for the detection of carbapenem resistant microbes over penicillinase can be enhanced by the further addition of a β-lactamase inhibitor (e.g. tazobactam) derived fragment to the R¹ position of the biosensor as disclosed here.

AmpC or cephalosporinases exhibits a greater hydrolysis for cephalosporins in comparison to benzylpenicillin (e.g., CMY-family AmpC).

Oxacillinase enzyme is able to hydrolyze cloxacillin or oxacillin. It is a wide group of ββ-lactamase and some of them can hydrolyze carbapenem such as e.g. OXA-48 or OXA-23.

On the other hand, ESBLs, some cephalosporinases and carbapenemases, when produced by bacteria, make them resistant to 3rd generation cephalosporins (C3GR). Thus, the selective detection of ESBL producing microbes over penicillinases can be achieve by having the first material derived from the third generation cephalosporins.

In some embodiments, the third material is derived from one or more of the following β-lactamase inhibitors: an AmpC inhibitor, a serine β-lactamase inhibitor, and an ESBL inhibitor.

In some embodiments, the third material is derived from an AmpC inhibitor. In some embodiments, the third material is derived from a serine β-lactamase inhibitor in an amount sufficient to inhibit ESBL and an OSBL but not a class A serine carbapenemase. In some embodiments, the third material is derived from an AmpC inhibitor and a serine β-lactamase inhibitor in an amount sufficient to inhibit ESBL and an OSBL but not a class A serine carbapenemase. In some embodiments, the third material is derived from an ESBL inhibitor.

As used herein, the term “AmpC inhibitor” refers to an agent that inhibits the enzymatic activity of AmpC at a particular concentrations, but not the enzymatic activity of serine carbapenemases, metallo-β-lactamases, OSBL and ESBL. Non-limiting examples of an AmpC inhibitor include cloxacillin

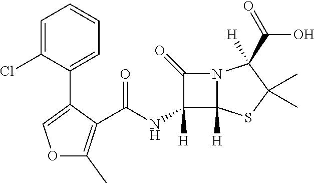 [SMILES: Cc1occ(-c2ccccc2Cl)c1C(=O)N[C@@H]1C(=O)N2[C@@H]1SC(C)(C)[C@@H]2C(=O)O]

(VWR, Pennsylvania, USA), salt forms of cloxacillin, syn2190 (NAEJA Pharmaceutical, Inc., Edmonton, Alberta, Canada), salt forms of cloxacillin (such as a sodium or potassium salt form of cloxacillin), aztreonam

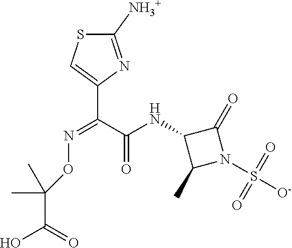 [SMILES: C[C@H]1[C@H](NC(=O)/C(=N\OC(C)(C)C(=O)O)c2csc([NH3+])n2)C(=O)N1S(=O)(=O)[O-]]

(VWR, Pennsylvania, U.S.A.) and boronic acid and derivatives thereof (Focus Synthesis LLC, San Diego, Calif.), and a combination thereof. In one embodiment, the AmpC inhibitor is cloxacillin.

In some embodiments, the biosensor having the fragment of an AmpC inhibitor is used at a concentration of about 1 μM to about 100 mM, about 1 μM to about 75 mM, about 1 μM to about 50 mM, about 1 μM to about 25 mM, about 1 μM to about 10 mM, or about 1 μM to about 5 mM. In some embodiments, the biosensor having the fragment of an AmpC inhibitor is used at a concentration of 100 μM to about 4 mM, about 200 μM to about 4 mM, 500 μM to about 4 mM, about 750 μM to about 4 mM, or about 1 mM to about 4 mM. In some embodiments, the biosensor having the fragment of an AmpC inhibitor is used at a concentration of about 500 μM to about 3 mM, about 1 mM to about 3 mM, about 1.5 mM to about 3 mM, or about 2 mM to about 3 mM. In a particular embodiment, the AmpC inhibitor is cloxacillin. In some embodiments, the biosensor having the fragment of cloxacillin is used at a concentration of about 20 μM to about 5 mM.

As used herein, the term “serine β-lactamase inhibitor” refers to an agent that at particular concentrations inhibits the enzymatic activity of ESBLs and OSBLs, but not class a serine carbapenemases and AmpC. Non-limiting examples of a serine β-lactamase inhibitor include clavulanic acid (GlaxoSmithKline, UK), salt forms of clavulanic acid (such as a sodium salt form of clavulanic acid), tazobactum

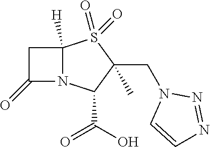 [SMILES: C[C@]1(Cn2ccnn2)[C@H](C(=O)O)N2C(=O)C[C@H]2S1(=O)=O]

(Wyeth Ayerst Research, New York, U.S.A.), sulbactam

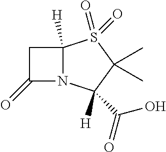

(Pfizer, N.Y., U.S.A.), and a combination thereof. In one embodiment, the serine β-lactamase inhibitor is clavulanic acid.

In some embodiments, a biosensor having a third material derived from clavulanic acid is used in an amount sufficient to inhibit an ESBL and an OSBL but not a class A serine carbapenemase and the concentration of the biosensor is about 10 μM to about 100 mM, about 10 μM to about 75 mM, about 10 μM to about 50 mM, about 10 μM to about 25 mM, about 10 μM to about 10 mM, or about 10 μM to about 5 mM.

In some embodiments, a biosensor having a third material derived from clavulanic acid is used in an amount sufficient to inhibit an ESBL and an OSBL but not a class A serine carbapenemase and the concentration of the biosensor is about 10 μM to about 2 mM, about 25 μM to about 2 mM, 50 μM to about 2 mM, about 75 μM to about 2 mM, or about 100 μM to about 2 mM.

In some embodiments, a biosensor having a third material derived from clavulanic acid is used in an amount sufficient to inhibit an ESBL and an OSBL but not a class A serine carbapenemase and the concentration of the biosensor is about 200 μM to about 2 mM, about 250 μM to about 2 mM, about 300 μM to about 2 mM, about 400 μM to about 2 mM, or about 500 μM to about 2 mM.

In some embodiments, a biosensor having a third material derived from clavulanic acid is used in an amount sufficient to inhibit an ESBL and an OSBL but not a class A serine carbapenemase and the concentration of the biosensor is about 1 μM to about 2 mM, about 1 μM to about 1.5 mM, about 1 μM to about 1 mM, about 1 μM to about 750 μM, about 1 μM to about 500 μM, about 1 μM to about 250 μM, or about 1 μM to 50 μM.

In some embodiments, a biosensor having a third material derived from clavulanic acid is used in an amount sufficient to inhibit an ESBL and an OSBL but not a class a serine carbapenemase and the concentration of the biosensor is of about 500 μM to about 1.5 mM.

In some embodiments, the biosensor has a third material derived from tazobactum (a serine β-lactamase inhibitor).

In some embodiments, a biosensor having a third material derived from tazobactum is used in an amount sufficient to inhibit an ESBL and an OSBL but not a class A serine carbapenemase and the concentration of the biosensor is of about 100 μM to about 1 mM, about 100 μM to about 750 μM, about 100 μM to about 500 μM, or about 100 μM to about 250 μM.

In some embodiments, the biosensor has a third material derived from sulbactam. In some embodiments, a biosensor having a third material derived from sulbactam is used in an amount sufficient to inhibit an ESBL and an OSBL but not a class A serine carbapenemase and the concentration of the biosensor is of about 100 μM to about 5 mM, about 100 μM to about 4 mM, about 100 μM to about 3 mM, about 100 μM to about 2 mM, or about 100 μM to about 1 mM. In some embodiments, the biosensor having a third material derived from sulbactam is used in an amount sufficient to inhibit an ESBL and an OSBL but not a class A serine carbapenemase and the concentration of the biosensor is of about 100 μM to about 750 μM, about 100 μM to about 500 μM, 100 μM to about 250 μM, about 100 μM to about 200 mM, or about 100 μM to about 150 μM.

In some embodiments, the third material is derived from an ESBL inhibitor. As used herein, the term “ESBL inhibitor” refers to an agent that at particular concentrations inhibits the enzymatic activity of ESBLs, but not the enzymatic activity of OSBLs. Non-limiting examples of an ESBL inhibitor include ceftazidime (GlaxoSmithKline, UK), a salt form of ceftazidime, cefotaxime (MP Biomedicals, Solon, Ohio, U.S.A.), a salt form of cefotaxime and a combination thereof.

In some embodiments, the ESBL inhibitor is ceftazidime. In some embodiments, the biosensor having a third material derived from ceftazidime is used at a concentration of about 1 mM to about 20 mM, about 1 mM to about 15 mM, about 1 mM to about 10 mM, about 1 mM to about 7 mM, about 1 mM to about 5 mM, or about 1 mM to about 2 mM.

In some embodiments, the ESBL inhibitor is cefotaxime. In some embodiments, the biosensor having a third material derived from cefotaxime is used at a concentration of about 1 mM to about 20 mM, about 1 mM to about 15 mM, about 1 mM to about 10 mM, about 1 mM to about 7 mM, about 1 mM to about 5 mM, or about 1 mM to about 2 mM.

In some embodiments, the third material is derived from antibiotic ceftazidime

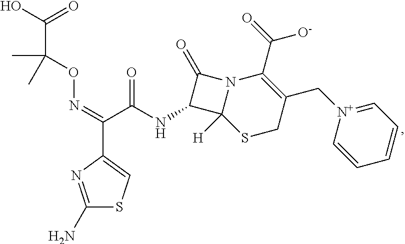 [SMILES: CC(C)(O/N=C(\C(=O)N[C@@H]1C(=O)N2C(C(=O)[O-])=C(C[n+]3ccccc3)CSC12)c1csc(N)n1)C(=O)O]

and cefotaxime

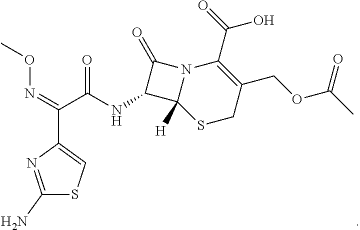 [SMILES: CO/N=C(\C(=O)N[C@@H]1C(=O)N2C(C(=O)O)=C(COC(C)=O)CS[C@H]12)c1csc(N)n1]

In some embodiments, the biosensor comprises a third material derived from the β-lactamase inhibitors suitable for the biosensor disclosed herein include

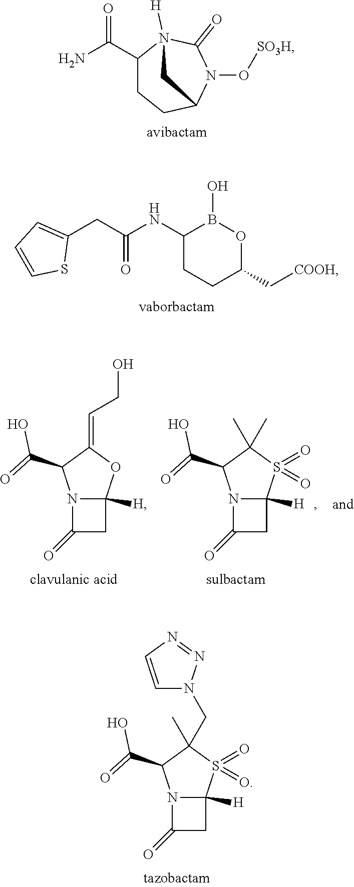

Clavulanate, sulbactam, tazobactam, avibactam, and vaborbactam are β-lactamase inhibitors that have little intrinsic antibacterial activity but inhibit the activity of a number of plasmid-mediated β-lactamases. Only avibactam inhibits the chromosomally mediated AmpC β-lactamases, and none inhibits the class B metallo-carbapenemases, such as New Delhi metallo-β-lactamase. Combination of these agents with ampicillin, amoxicillin, piperacillin, ceftolozane, ceftazidime, and meropenem results in antibiotics with an enhanced spectrum of activity against many, but not all, organisms containing plasmid-mediated β-lactamases. The addition of avibactam to ceftazidime and vaborbactam to meropenem results in enhanced activity against many, but not all, organisms producing carbapenemases. In addition, sulbactam and tazobactam inhibit the chromosomal β-lactamase of many Bacteroides species, extending the spectrum of coverage of combinations with these compounds to include Bacteroides as well.

In some embodiments, this disclosure provides methods for the rapid detection of particular β-lactamases using a detectable β-lactamase substrate and certain β-lactamase inhibitors, for example, serine carbapenemases, metallo-β-lactamases, AmpC, and extended-spectrum β-lactamases (ESBLs). Methods presented herein do not require the production of a bacterial cell extract and only require a small amount of a bacterial sample (e.g., less than 10¹⁰ CFU/ml of bacteria). In some embodiments, methods disclosed herein permit the detection of the presence of such β-lactamases in bacterial samples within as few as 2 to 10 minutes. Detection of the presence of the β-lactamases can provide information for the selection of the appropriate therapeutic regimen for a patient with a bacterial infection.

In some embodiments, a spacer links the β-lactamase inhibitor fragment and the β-lactam antibiotic fragment to each other. In some embodiments, the spacer links the two β-lactamase sensing components via an amide bond formed by NHS/EDC chemistry. In some embodiments, the spacer links the two β-lactamase sensing components via a disulfide (S—S) bond.

In some embodiments, the β-lactamase sensing components comprises β-lactamase inhibitor fragment -(amino-(spacer)x)y-β-lactam antibiotic fragment or β-lactam antibiotic fragment-(spacer)z-β-lactamase inhibitor fragment, wherein the spacer has from 2 to 50 atoms, x, y and z are integers from 5 to 15.

In some embodiments, the spacer links the β-lactamase inhibitor fragment and the β-lactam antibiotic fragment via a degradable bond that is selected from the group consisting of an ester bond, an amide bond, an imine bond, an acetal bond, a ketal bond, and combinations thereof.

In some embodiments, the spacer is selected from the group consisting of polyethylene glycol having 2-50 repeating units, ε-maleimidocaproic acid, para-aminobenzyloxy carbamater, and combinations thereof. In some embodiments, the spacer comprises polyamino acid having 2-30 amino acid residues. In some embodiments, the spacer comprises a linear polylysine, or polyglutamine.

### The Spectroscopic Probe (D)

The principle of producing optical response is based on their ability of a chromophore to co-exist as colorless neutral compounds or colored zwitterionic compound. The color change between the two states of the compound produces a detectable optical response. In some embodiments, the optical response produced by the biosensor is a color change from colored state to colorless state. In some embodiments, the optical response produced by the biosensor is a color change from colorless state to colored state. In some embodiments, the optical response produced by the biosensor is a change from non-fluorescent state to fluorescent state.

In some embodiments, when the biosensors disclosed herein are cleaved by one or more antimicrobial inactivating factors secreted by the drug resistant microbes, the spectroscopic probe is produced. In aqueous solution, the spectroscopic probe will exist as colorless dye or colored dye. The colorimetric profile of the spectroscopic probes are dependent on the electron donating/electron withdrawing properties of the substituents present on the aromatic rings, for example, R⁵, R⁶, R⁷, R⁸, R¹¹, R¹², R¹³, R¹⁴, R¹⁵, R¹⁶, and R¹⁷, as in Formulae (9) and (10). Accordingly, the detection wavelength and minimum detection concentration of the spectroscopic probes can be modulated by altering the number and positioning of the electron donating/electron withdrawing on the aromatic rings of the spectroscopic probes.

In some embodiments, the spectroscopic probe D is selected from the group consisting of a fluorophore, a chromophore, an infrared chromophore, a visible light chromophore, and combinations thereof. In some embodiments, D is derived from a colored dye. In some embodiments, D is a colorless component derived from a leuco dye, wherein reaction of the biosensor with the inactivating factor produces a fluorophore, a chromophore, an infrared chromophore, a visible light chromophore, and combinations thereof.

In some embodiments, the chromophore absorbs wavelengths of the visible light spectrum ranging from 400 nm to 500 nm. In some embodiments, the chromophore absorbs wavelengths of the visible light spectrum ranging from 500 nm to 600 nm. In some embodiments, the chromophore absorbs wavelengths of the visible light spectrum ranging from 600 nm to 700 nm. In some embodiments, the chromophore has the emission wavelength in the visible light spectrum ranging from 400 nm to 1300 nm. In some embodiments, the chromophore has the emission wavelength in the visible light spectrum ranging from 400 nm to 750 nm. Chromophores that either absorb or emit in the yellow, red and near infrared spectrum are preferred, i.e., 550-1300 nm.

In some embodiments, D comprises a structure derived from a xanthene chromophore. In some embodiments, D comprises a structure derived from a fluorescein, a rhodol, or a rhodamine chromophore. In some embodiments, D is a colorless component derived from a leuco dye, wherein reaction of the biosensor with the inactivating factor produces a fluorescein, a rhodol, or a rhodamine chromophore. In some embodiments, the leuco dye is a triarylmethane dye. In some embodiments, the leuco dye is a fluoran dye.

In some embodiments, D comprises a colored component having

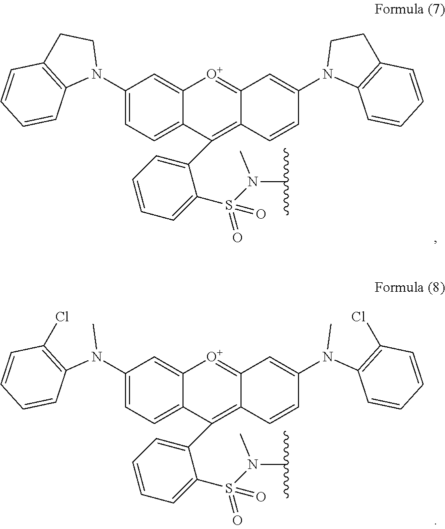 [SMILES: *N(C)S(=O)(=O)c1ccccc1-c1c2ccc(N3CCc4ccccc43)cc2[o+]c2cc(N3CCc4ccccc43)ccc12 ; *N(C)S(=O)(=O)c1ccccc1-c1c2ccc(N(C)c3ccccc3Cl)cc2[o+]c2cc(N(C)c3ccccc3Cl)ccc12]

In some embodiments, D comprises a colorless leuco dye component having

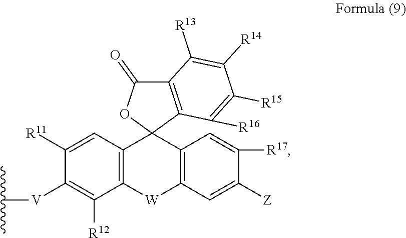

wherein

### W is O, N, S or —CH₂—;

Z is —NR⁹R¹⁰, —O—CH₂-Ph, or V;
R⁹ and R¹⁰ is a substituent each independently selected from the group of H, substituted and unsubstituted C₁-C₁₂ alkyl group, substituted and unsubstituted C₁-C₁₂ alkenyl group, substituted and unsubstituted C₁-C₁₂ alkynyl group, substituted and unsubstituted aryl group, fluoroalkyl, substituted and unsubstituted carbocyclyl, substituted and unsubstituted carbocyclylalkyl, substituted and unsubstituted aralkyl, substituted and unsubstituted heterocycloalkyl, substituted and unsubstituted heterocycloalkylalkyl, substituted and unsubstituted heteroaryl, and substituted and unsubstituted heteroarylalkyl; R¹¹, R¹², R¹³, R¹⁴, R¹⁵, R¹⁶, and R¹⁷ is each independently selected from the group of H, Cl, F, Br, CN, NO₂, —NR⁹R¹⁰, C1-C6 alkyl group, and C1-C6 alkoxyl group; and V represents the point of attachment of the fragmentable group FG.

In some embodiments, D comprises a structure derived from a thiazine, an oxazine, or a phenazine chromophore. In some embodiments, D comprises a colorless leuco dye component having

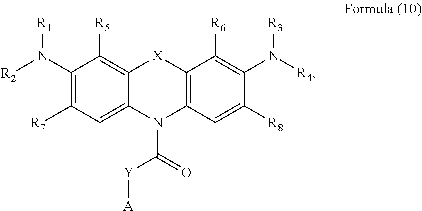

wherein

### X is —CH₂, O, N, or S;

Y is a bond, O or N;
R¹, R², R³, R⁴ are each independently selected from the group consisting of H, substituted and unsubstituted C₁-C₁₂ alkyl group, substituted and unsubstituted C₁-C₁₂ alkenyl group, substituted and unsubstituted C₁-C₁₂ alkynyl group, and substituted and unsubstituted aryl group; R⁵, R⁶, R⁷, and R⁸ is each independently selected from the group consisting of H, Cl, F, Br, CN, NO₂, —NR⁹R¹⁰, C₁-C₆ alkyl group, and C₁-C₆ alkoxyl group; and A represents the point of attachment of the fragmentable group FG.

In some embodiments, D comprises a structure derived from methylene blue. In some embodiments, D comprises a structure having

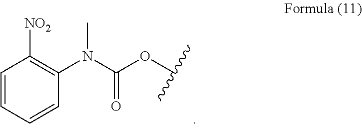 [SMILES: *OC(=O)N(C)c1ccccc1[N+](=O)[O-]]

### SPECIFIC EMBODIMENTS

In some embodiments, the biosensor is a compound selected from those disclosed in Table 1 that comprise Formula (7) or Formula (8).

TABLE 1
Embodiments of biosensors derived from rhodamines.

| Entry No. | Compound | Chemical Name | Optical Response |
|---|---|---|---|
| MF-0001 | 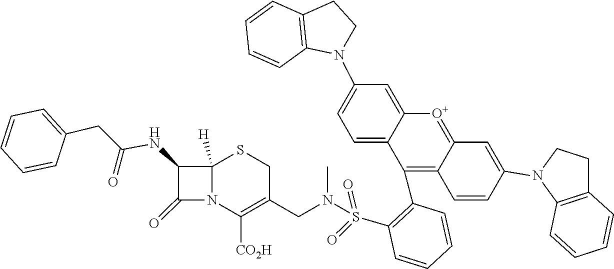 [SMILES: CN(CC1=C(C(=O)O)N2C(=O)[C@@H](NC(=O)Cc3ccccc3)[C@H]2SC1)S(=O)(=O)c1ccccc1-c1c2ccc(N3CCc4ccccc43)cc2[o+]c2cc(N3CCc4ccccc43)ccc12] | 9-(2-(N-(((7R)-2-carboxy- 8-oxo-7-(2-phenylacetamido)- 5-thia-1-azabicyclo[4.2.0]oct- 2-en-3-yl)methyl)-N- methylsulfamoyl)phenyl)- 3,6-di(indolin-1-yl)xanthylium | Cyan to Colorless |
| MF-0002 | 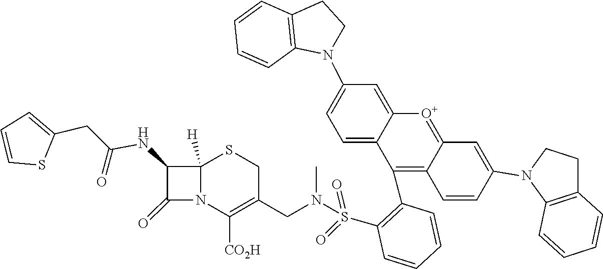 [SMILES: CN(CC1=C(C(=O)O)N2C(=O)[C@@H](NC(=O)Cc3cccs3)[C@H]2SC1)S(=O)(=O)c1ccccc1-c1c2ccc(N3CCc4ccccc43)cc2[o+]c2cc(N3CCc4ccccc43)ccc12] | 9-(2-(N-(((7R)-2-carboxy-8- oxo-7-(2-(thiophen-2-yl)acetamido)- 5-thia-1-azabicyclo[4.2.0]oct-2-en- 3-yl)methyl)-N-methylsulfamoyl) phenyl)-3,6-di(indolin-1-yl)xanthylium | Cyan to Colorless |
| MF-0003 | 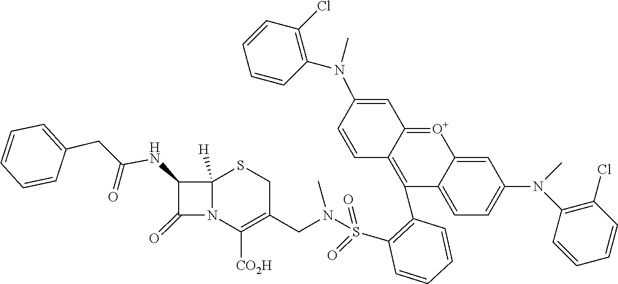 [SMILES: CN(c1ccc2c(-c3ccccc3S(=O)(=O)N(C)CC3=C(C(=O)O)N4C(=O)[C@@H](NC(=O)Cc5ccccc5)[C@H]4SC3)c3ccc(N(C)c4ccccc4Cl)cc3[o+]c2c1)c1ccccc1Cl] | 9-((1r)-2-(N-(((7R)-2-carboxy-8-oxo- 7-(2-phenylacetamido)-5-thia-1- azabicyclo[4.2.0]oct-2-en-3-yl) methyl)-N-methylsulfamoyl)phenyl)- 3,6-bis((2-chlorophenyl) (methyl)amino)xanthylium | Magenta to colorless |
| MF-0004 | 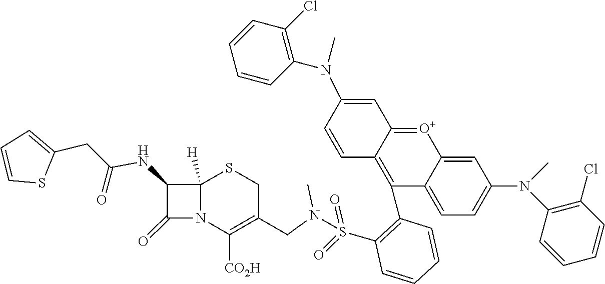 [SMILES: CN(c1ccc2c(-c3ccccc3S(=O)(=O)N(C)CC3=C(C(=O)O)N4C(=O)[C@@H](NC(=O)Cc5cccs5)[C@H]4SC3)c3ccc(N(C)c4ccccc4Cl)cc3[o+]c2c1)c1ccccc1Cl] | 9-((1r)-2-(N-(((7R)-2-carboxy-8-oxo- 7-(2-(thiophen-2-yl)acetamido)-5-thia-1- azabicyclo[4.2.0]oct-2-en-3-yl)methyl)-N- methylsulfamoyl)phenyl)-3,6-bis((2- chlorophenyl)(methyl)amino)xanthylium | Magenta to colorless |
| MF-0005 | 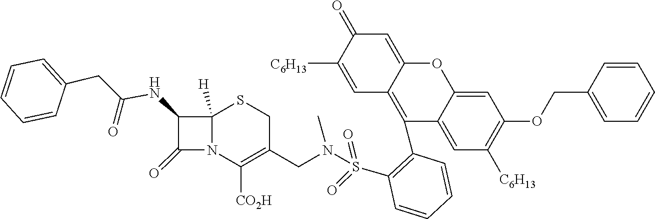 | (7R)-3-(((2-(6-(benzyloxy)-2,7-dihexyl-3- oxo-3H-xanthen-9-yl)-N-methylphenyl) sulfonamido)methyl)-8-oxo-7-(2- phenylacctamido)-5-thia-1-azabicyclo [4.2.0]oct-2-ene-2-carboxylic acid | Yellow to Colorless |
| MF-0006 | 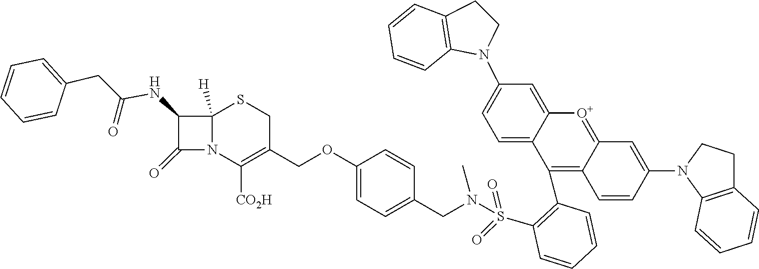 [SMILES: CN(Cc1ccc(OCC2=C(C(=O)O)N3C(=O)[C@@H](NC(=O)Cc4ccccc4)[C@H]3SC2)cc1)S(=O)(=O)c1ccccc1-c1c2ccc(N3CCc4ccccc43)cc2[o+]c2cc(N3CCc4ccccc43)ccc12] | 9-(2-(N-(4-(((7R)-2-carboxy-8-oxo- 7-(2-phenylacetamido)-5-thia-1- azabicyclo[4.2.0]oct-2-en-3-yl)methoxy) benzyl)-N-methylsulfamoyl)phenyl)- 3,6-di(indolin-1-yl)xanthylium | Cyan to colorless |
| MF-0007 | 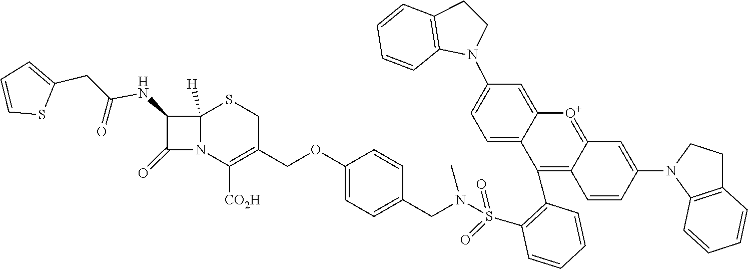 [SMILES: CN(Cc1ccc(OCC2=C(C(=O)O)N3C(=O)[C@@H](NC(=O)Cc4cccs4)[C@H]3SC2)cc1)S(=O)(=O)c1ccccc1-c1c2ccc(N3CCc4ccccc43)cc2[o+]c2cc(N3CCc4ccccc43)ccc12] | 9-(2-(N-(4-(((7R)-2-carboxy-8-oxo-7-(2- (thiophen-2-yl)acctamido)-5-thia-1- azabicyclo[4.2.0]oct-2-en-3-yl)methoxy) benzyl)-N-methylsulfamoyl)phenyl)- 3,6-di(indolin-l-yl)xanthylium | Cyan to colorless |
| MF-0008 | 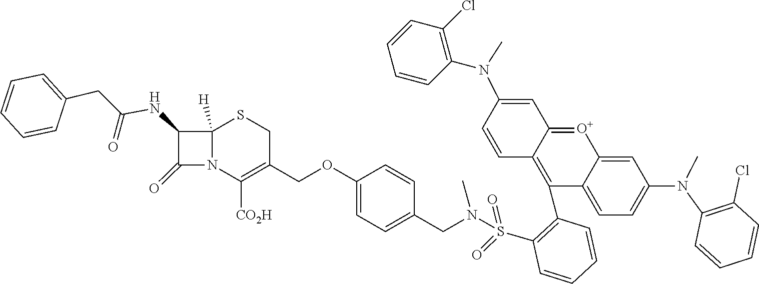 [SMILES: CN(c1ccc2c(-c3ccccc3S(=O)(=O)N(C)Cc3ccc(OCC4=C(C(=O)O)N5C(=O)[C@@H](NC(=O)Cc6ccccc6)[C@H]5SC4)cc3)c3ccc(N(C)c4ccccc4Cl)cc3[o+]c2c1)c1ccccc1Cl] | 9-((1r)-2-(N-(4-(((7R)-2-carboxy-8-oxo- 7-(2-phenylacetamido)-5-thia-1-azabicyclo [4.2.0]oct-2-en-3-yl)methoxy)benzyl)-N- methylsulfamoyl)phenyl)-3,6-bis((2- chlorophenyl)(methyl)amino)xanthylium | Magenta to colorless |
| MF-0009 | 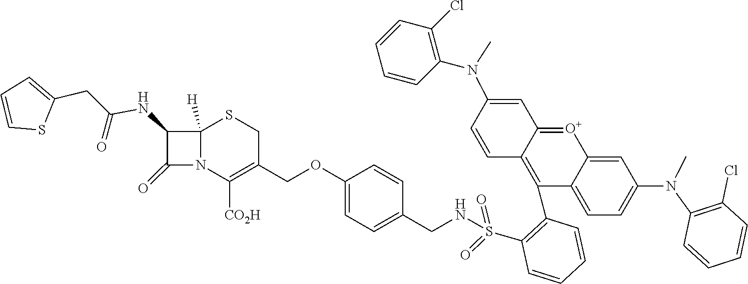 [SMILES: CN(c1ccc2c(-c3ccccc3S(=O)(=O)NCc3ccc(OCC4=C(C(=O)O)N5C(=O)[C@@H](NC(=O)Cc6cccs6)[C@H]5SC4)cc3)c3ccc(N(C)c4ccccc4Cl)cc3[o+]c2c1)c1ccccc1Cl] | 9-((1r)-2-(N-(4-(((7R)-2-carboxy-8- oxo-7-(2-(thiophen-2-yl)acetamido)- 5-thia-1-azabicyclo[4.2.0]oct-2-en- 3-yl)methoxy)benzyl)sulfamoyl) phenyl)-3,6-bis((2-chlorophenyl) (methyl)amino)xanthylium | Magenta to colorless |
| MF-0010 | 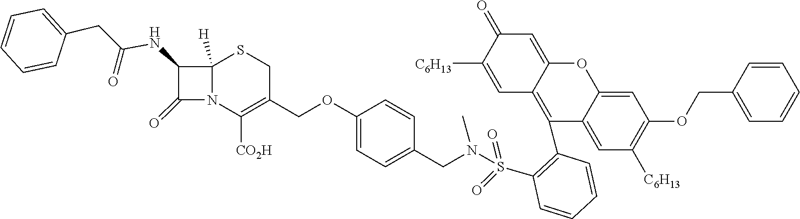 | (7R)-3-((4-(((2-(6-(benzyloxy)-2,7- dihexyl-3-oxo-3H-xanthen-9-yl)-N- methylphenyl)sulfonamido)methyl)phenoxy) methyl)-8-oxo-7-(2-phenylacetamido)-5- thia-1-azabicyclo[4.2.0]oct-2-ene- 2-carboxylic acid | Yellow to Colorless |
| MF-0030 | 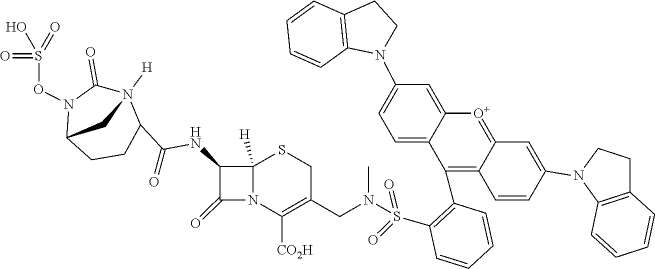 | 9-(2-(N-(((7R)-2-carboxy-8-oxo-7-((1S,5S)- 7-oxo-6-(sulfooxy)-114,6- diazabicyclo[3.2.1]octane-2- carboxamido)-5-thia-1-azabicyclo [4.2.0]oct-2-en-3-yl)methyl)-N- methylsulfamoyl)phenyl)- 3,6-di(indolin-1-yl)xanthylium | Cyan to Colorless |
| MF-0031 | 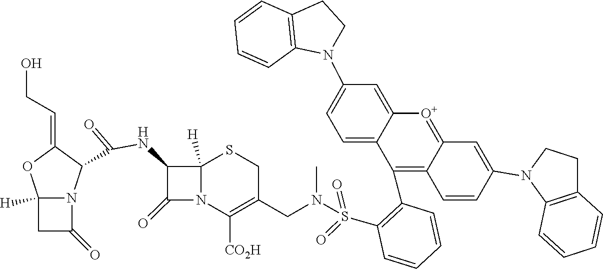 [SMILES: CN(CC1=C(C(=O)O)N2C(=O)[C@@H](NC(=O)[C@H]3/C(=C/CO)O[C@@H]4CC(=O)N43)[C@H]2SC1)S(=O)(=O)c1ccccc1-c1c2ccc(N3CCc4ccccc43)cc2[o+]c2cc(N3CCc4ccccc43)ccc12] | 9-(2-(N-(((7R)-2-carboxy-7-((2R,5R,Z)-3-(2- hydroxyethylidene)-7-oxo-4-oxa-1- azabicyclo[3,2.0]heptane-2-carboxamido)-8- oxo-5-thia-1-azabicyclo[4.2.0]oct-2-en- 3-yl)methyl)-N-methylsulfamoyl) phenyl)-3,6-di(indolin-1-yl)xanthylium | Cyan to Colorless |
| MF-0032 | 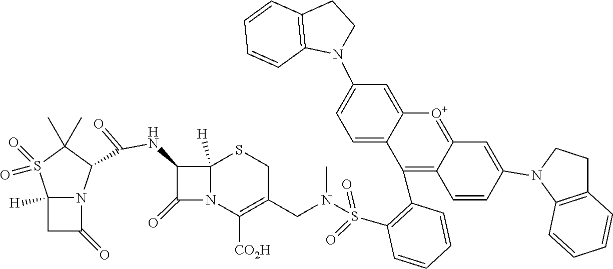 [SMILES: CN(CC1=C(C(=O)O)N2C(=O)[C@@H](NC(=O)[C@@H]3N4C(=O)C[C@H]4S(=O)(=O)C3(C)C)[C@H]2SC1)S(=O)(=O)c1ccccc1-c1c2ccc(N3CCc4ccccc43)cc2[o+]c2cc(N3CCc4ccccc43)ccc12] | 9-(2-(N-(((7R)-2-carboxy-7-((2S,5R)-3,3- dimethyl-4,4-dioxido-7-oxo-4-thia-1- azabicyclo[3.2.0]heptane-2-carboxamido)-8- oxo-5-thia-1-azabicyclo[4.2,0]oct-2-en- 3-yl)methyl)-N-methylsulfamoyl)phenyl)- 3,6-di(indolin-1-yl)xanthylium | Cyan to Colorless |
| MF-0033 | 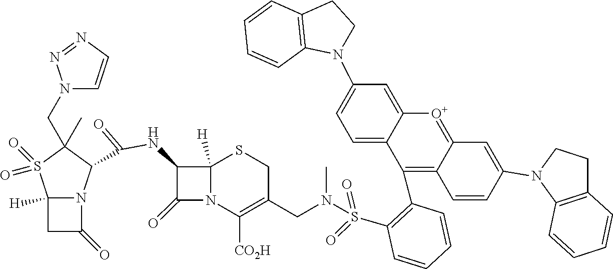 [SMILES: CN(CC1=C(C(=O)O)N2C(=O)[C@@H](NC(=O)[C@@H]3N4C(=O)C[C@H]4S(=O)(=O)C3(C)Cn3ccnn3)[C@H]2SC1)S(=O)(=O)c1ccccc1-c1c2ccc(N3CCc4ccccc43)cc2[o+]c2cc(N3CCc4ccccc43)ccc12] | 9-(2-(N-(((7R)-7-((2S,3R,5R)-3-((1H-1,2,3- triazol-1-yl)methyl)-3-methyl- 4,4-dioxido-7-oxo-4-thia-1- azabicyclo[3.2.0]heptane-2-carboxamido)-2- carboxy-8-oxo-5-thia-1-azabicyclo[4.2.0] oct-2-en-3-yl)methyl)-N-methylsulfamoyl) phenyl)-3,6-di(indolin-1-yl)xanthylium | Cyan to Colorless |
| MF-0034 | 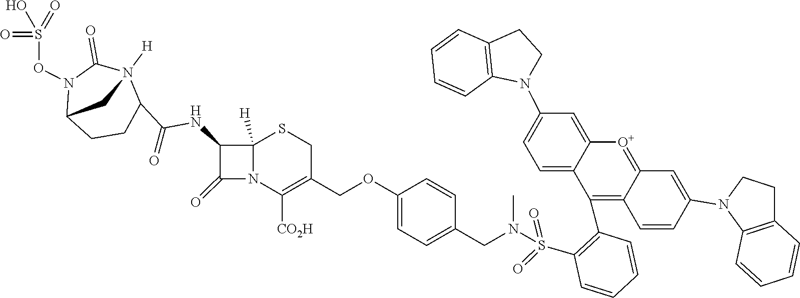 | 9-(2-(N-(4-(((7R)-2-carboxy-8-oxo- 7-((1S,5S)-7-oxo-6-(sulfooxy)-114,6- diazabicyclo[3.2.1]octane-2-carboxamido)-5- thia-1-azabicyclo[4.2.0]oct-2-en-3-yl) methoxy)benzyl)-N-methylsulfamoyl) phenyl)-3,6-di(indolin-1-yl)xanthylium | Cyan to Colorless |
| MF-0035 | 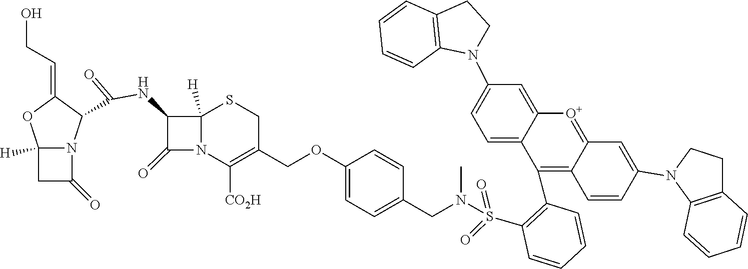 [SMILES: CN(Cc1ccc(OCC2=C(C(=O)O)N3C(=O)[C@@H](NC(=O)[C@H]4/C(=C/CO)O[C@@H]5CC(=O)N54)[C@H]3SC2)cc1)S(=O)(=O)c1ccccc1-c1c2ccc(N3CCc4ccccc43)cc2[o+]c2cc(N3CCc4ccccc43)ccc12] | 9-(2-(N-(4-(((7R)-2-carboxy-7- ((2R,5R,Z)-3-(2-hydroxyethylidene)- 7-oxo-4-oxa-1-azabicyclo[3,2.0] heptane-2-carboxamido)-8-oxo-5-thia-1- azabicyclo[4.2.0]oct-2-en-3-yl)methoxy) benzyl)-N-methylsulfamoyl)phenyl)- 3,6-di(indolin-1-yl)xanthylium | Cyan to Colorless |
| MF-0036 | 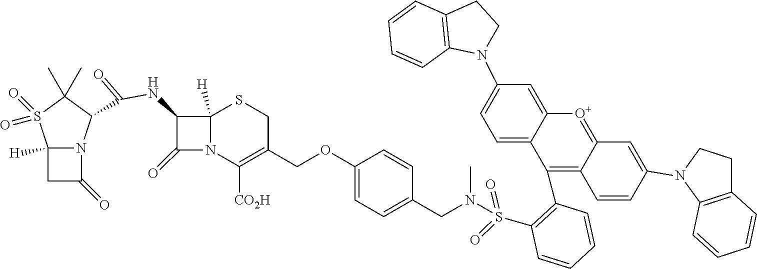 [SMILES: CN(Cc1ccc(OCC2=C(C(=O)O)N3C(=O)[C@@H](NC(=O)[C@@H]4N5C(=O)C[C@H]5S(=O)(=O)C4(C)C)[C@H]3SC2)cc1)S(=O)(=O)c1ccccc1-c1c2ccc(N3CCc4ccccc43)cc2[o+]c2cc(N3CCc4ccccc43)ccc12] | 9-(2-(N-(4-(((7R)-2-carboxy-7-((2S,5R)-3,3- dimethyl-4,4-dioxido-7-oxo-4-thia-1- azabicyclo[3,2.0]heptane-2-carboxamido)-8- oxo-5-thia-1-azabicyclo[4.2,0]oct-2-en-3-yl) methoxy)benzyl)-N-methylsulfamoyl) phenyl)-3,6-di(indolin-l-yl)xanthylium | Cyan to Colorless |
| MF-0037 | 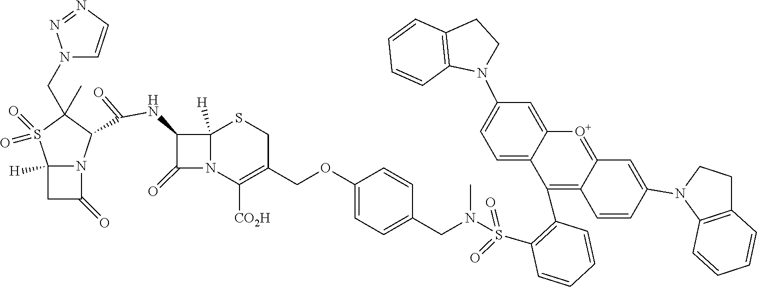 [SMILES: CN(Cc1ccc(OCC2=C(C(=O)O)N3C(=O)[C@@H](NC(=O)[C@@H]4N5C(=O)C[C@H]5S(=O)(=O)C4(C)Cn4ccnn4)[C@H]3SC2)cc1)S(=O)(=O)c1ccccc1-c1c2ccc(N3CCc4ccccc43)cc2[o+]c2cc(N3CCc4ccccc43)ccc12] | 9-(2-(N-(4-(((7R)-7-((2S,3R,5R)-3- ((1H-1,2,3-triazol-1-yl)methyl)-3- methyl-4,4-dioxido-7-oxo-4-thia-1- azabicyclo[3.2.0]heptane-2- carboxamido)-2-carboxy-8-oxo-5- thia-1-azabicyclo[4.2.0]oct-2-en- 3-yl)methoxy)benzyl)-N-methylsulfamoyl) phenyl)-3,6-di(indolin-1-yl)xanthylium | Cyan to Colorless |
| MF-0038 | 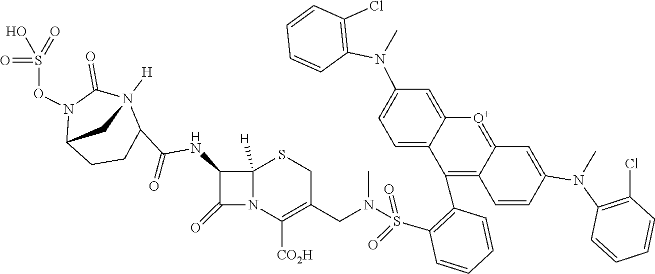 | 9-(2-(N-(((7R)-2-carboxy-8-oxo-7-((1S,5S)- 7-oxo-6-(sulfooxy)-114,6-diazabicyclo [3.2.1]octane-2-carboxamido)-5-thia-1- azabicyclo[4.2.0]oct-2-en-3-yl) methyl)-N-methylsulfamoyl)phenyl)-3,6- bis((2-chlorophenyl)(methyl)amino) xanthylium | Magenta to colorless |
| MF-0039 | 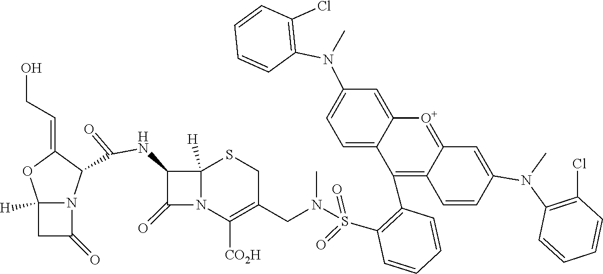 [SMILES: CN(c1ccc2c(-c3ccccc3S(=O)(=O)N(C)CC3=C(C(=O)O)N4C(=O)[C@@H](NC(=O)[C@H]5/C(=C/CO)O[C@@H]6CC(=O)N65)[C@H]4SC3)c3ccc(N(C)c4ccccc4Cl)cc3[o+]c2c1)c1ccccc1Cl] | 9-(2-(N-(((7R)-2-carboxy-7-((2R,5R,Z)-3-(2- hydroxyethylidene)-7-oxo-4-oxa-1- azabicyclo[3.2.0]heptane-2-carboxamido)-8- oxo-5-thia-1-azabicyclo[4.2.0]oct-2-en- 3-yl)methyl)-N-methylsulfamoyl) phenyl)-3,6-bis((2-chlorophenyl) (methyl)amino)xanthylium | Magenta to colorless |
| MF-0040 | 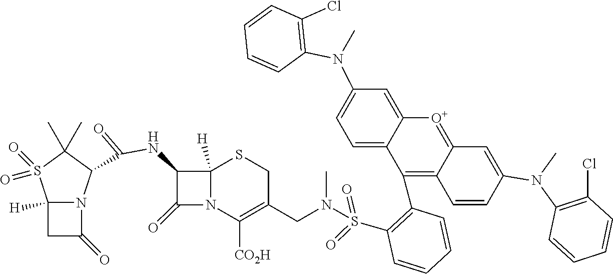 [SMILES: CN(c1ccc2c(-c3ccccc3S(=O)(=O)N(C)CC3=C(C(=O)O)N4C(=O)[C@@H](NC(=O)[C@@H]5N6C(=O)C[C@H]6S(=O)(=O)C5(C)C)[C@H]4SC3)c3ccc(N(C)c4ccccc4Cl)cc3[o+]c2c1)c1ccccc1Cl] | 9-(2-(N-(((7R)-2-carboxy-7-((2S,5R)-3,3- dimethyl-4,4-dioxido-7-oxo-4-thia-1- azabicyclo[3,2.0]heptane-2-carboxamido)-8- oxo-5-thia-1-azabicyclo[4.2,0]oct-2-en- 3-yl)methyl)-N-methylsulfamoyl)phenyl)- 3,6-bis((2-chlorophenyl)(methyl)amino) xanthylium | Magenta to colorless |
| MF-0041 | 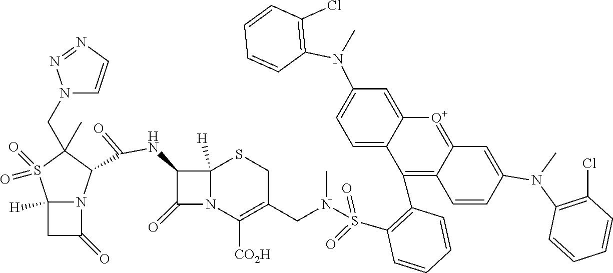 [SMILES: CN(c1ccc2c(-c3ccccc3S(=O)(=O)N(C)CC3=C(C(=O)O)N4C(=O)[C@@H](NC(=O)[C@@H]5N6C(=O)C[C@H]6S(=O)(=O)C5(C)Cn5ccnn5)[C@H]4SC3)c3ccc(N(C)c4ccccc4Cl)cc3[o+]c2c1)c1ccccc1Cl] | 9-(2-(N-(((7R)-7-((2S,3R,5R)-3-((lH-l,2,3- triazol-1-yl)methyl)-3-methyl-4,4- dioxido-7-oxo-4-thia-1-azabicyclo[3,2.0] heptane-2-carboxamido)-2-carboxy- 8-oxo-5-thia-1-azabicyclo[4.2.0]oct-2-en- 3-yl)methyl)-N-methylsulfamoyl) phenyl)-3,6-bis((2-chlorophenyl)(methyl) amino)xanthylium | Magenta to colorless |
| MF-0042 | 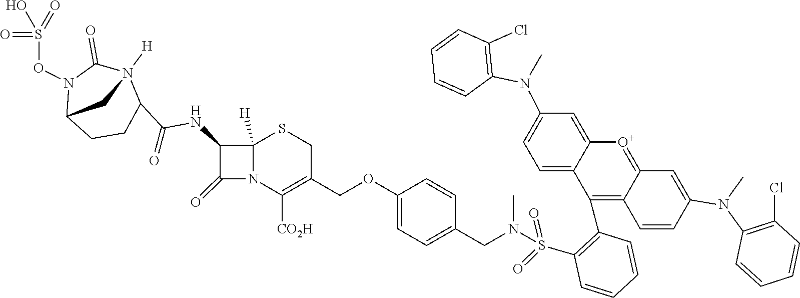 | 9-(2-(N-(4-(((7R)-2-carboxy-8-oxo- 7-((1S,5S)-7-oxo-6-(sulfooxy)-114,6- diazabicyclo[3.2.1]octane-2-carboxamido)- 5-thia-1-azabicyclo[4.2.0]oct-2-en- 3-yl)methoxy)benzyl)-N-methylsulfamoyl) phenyl)-3,6-bis((2-chlorophenyl) (methyl)amino)xanthyiium | Magenta to colorless |
| MF-0043 | 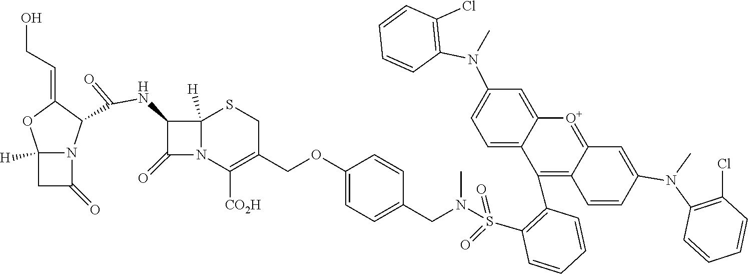 [SMILES: CN(c1ccc2c(-c3ccccc3S(=O)(=O)N(C)Cc3ccc(OCC4=C(C(=O)O)N5C(=O)[C@@H](NC(=O)[C@H]6/C(=C/CO)O[C@@H]7CC(=O)N76)[C@H]5SC4)cc3)c3ccc(N(C)c4ccccc4Cl)cc3[o+]c2c1)c1ccccc1Cl] | 9-(2-(N-(4-(((7R)-2-carboxy-7- ((2R,5R,Z)-3-(2-hydroxyethylidene)- 7-oxo-4-oxa-1-azabicyclo[3,2.0] heptane-2-carboxamido)-8-oxo-5-thia-1- azabicyclo[4.2.0]oct-2-en-3-yl) methoxy)benzyl)-N-methylsulfamoyl) phenyl)-3,6-bis((2-chlorophenyl) (methyl)amino)xanthylium | Magenta to colorless |
| MF-0044 | 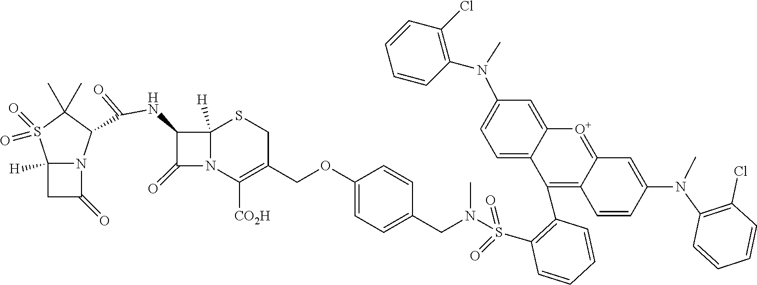 [SMILES: CN(c1ccc2c(-c3ccccc3S(=O)(=O)N(C)Cc3ccc(OCC4=C(C(=O)O)N5C(=O)[C@@H](NC(=O)[C@@H]6N7C(=O)C[C@H]7S(=O)(=O)C6(C)C)[C@H]5SC4)cc3)c3ccc(N(C)c4ccccc4Cl)cc3[o+]c2c1)c1ccccc1Cl] | 9-(2-(N-(4-(((7R)-2-carboxy-7-((2S,5R)-3,3- dimethyl-4,4-dioxido-7-oxo-4-thia-1- azabicyclo[3.2.0]heptane-2-carboxamido)-8- oxo-5-thia-1-azabicyclo[4.2,0]oct-2-en-3-yl) methoxy)benzyl)-N-methylsulfamoyl) phenyl)-3,6-bis((2-chlorophenyl (methyl)amino)xanthylium | Magenta to colorless |
| MF-0045 | 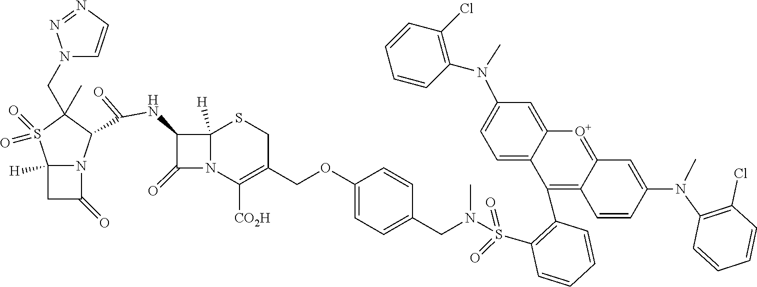 [SMILES: CN(c1ccc2c(-c3ccccc3S(=O)(=O)N(C)Cc3ccc(OCC4=C(C(=O)O)N5C(=O)[C@@H](NC(=O)[C@@H]6N7C(=O)C[C@H]7S(=O)(=O)C6(C)Cn6ccnn6)[C@H]5SC4)cc3)c3ccc(N(C)c4ccccc4Cl)cc3[o+]c2c1)c1ccccc1Cl] | 9-(2-(N-(4-(((7R)-7-((2S,3R,5R)-3- ((1H-1,2,3-triazol-1-yl)methyl)-3-methyl- 4,4-dioxido-7-oxo-4-thia-1- azabicyclo[3,2.0]heptane-2-carboxamido)-2- carboxy-8-oxo-5-thia-1-azabicyclo [4.2.0]oct-2-en-3-yl)methoxy)benzyl)-N- methylsulfamoyl)phenyl)-3,6-bis((2- chlorophenyl)(methyl)amino)xanthylium | Magenta to colorless |

In some embodiments, the biosensor is a compound selected from those disclosed in Table 2 that comprise Formula (9).

TABLE 2
Embodiments of biosensors derived from leuco rhodols.

| Entry No. | Compound | Chemical Name | Optical Response |
|---|---|---|---|
| MF-0011 | 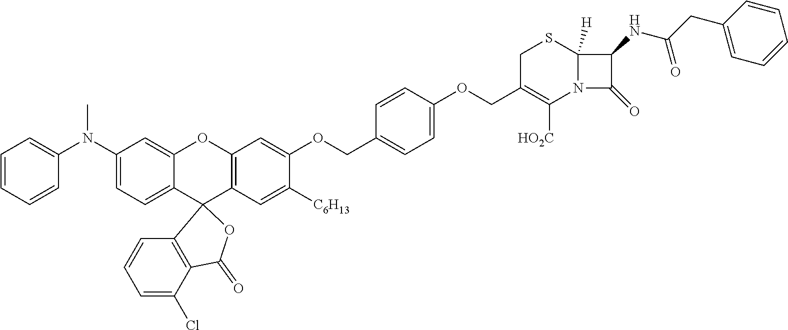 | (7S)-3-((4-(((2′-hexyl-6′- (methyl(phenyl)amino)-3- oxo-3H- spiro[isobenzofuran-1,9′- xanthen]-3′- yl)oxy)methyl)phenoxy) methyl)-8-oxo-7-(2- phenylacetamido)-5-thia-1- azabicyclo[4.2.0]oct-2- ene-2-carboxylic acid | Colorless to Red |
| MF-0012 | 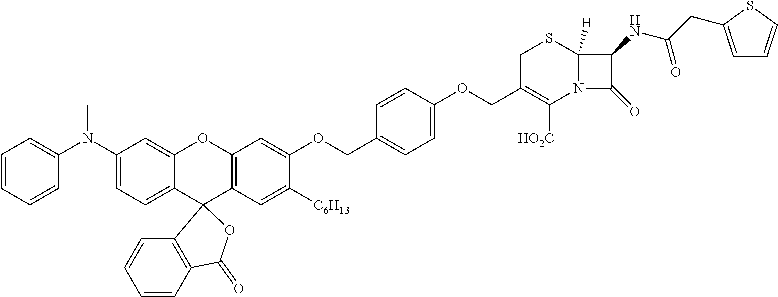 | (7S)-3-((4-(((2′-hexyl-6′- (methyl(phenyl)amino)-3- oxo-3H- spiro[isobenzofuran-1,9′- xanthen]-3′- yl)oxy)methyl)phenoxy) methyl)-8-oxo-7-(2- (thiophen-2-yl) acetamido)-5-thia-1- azabicyclo[4.2.0]oct-2- ene-2-carboxylic acid | Colorless to Red |
| MF-0013 | 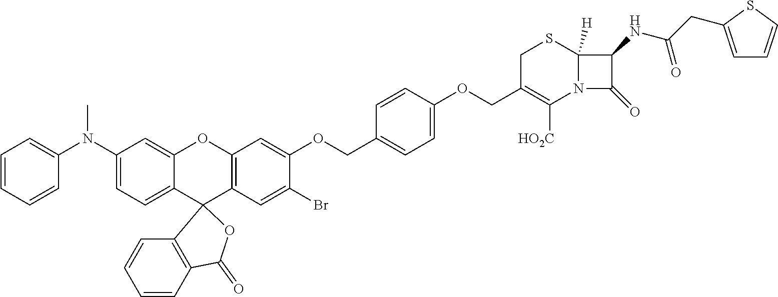 [SMILES: CN(c1ccccc1)c1ccc2c(c1)Oc1cc(OCc3ccc(OCC4=C(C(=O)O)N5C(=O)[C@H](NC(=O)Cc6cccs6)[C@@H]5SC4)cc3)c(Br)cc1C21OC(=O)c2ccccc21] | (7S)-3-((4-(((2′-bromo-6′- (methyl(phenyl)amino)-3- oxo-3H- spiro[isobenzofuran-1,9′- xanthen]-3′- yl)oxy)methyl)phenoxy) methyl)-8-oxo-7-(2- (thiophen-2-yl) acetamido)-5-thia-1- azabicyclo[4.2.0]oct-2- ene-2-carboxylic acid | Colorless to Red |
| MF-0014 | 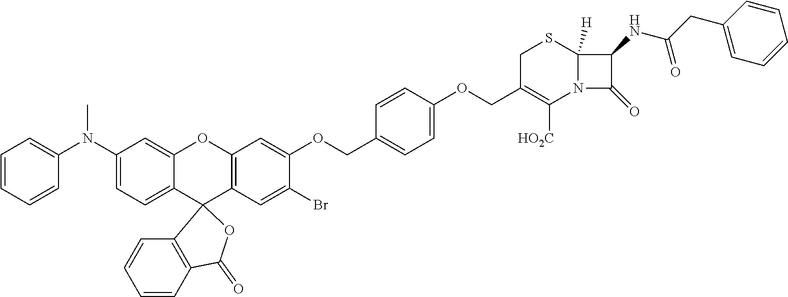 [SMILES: CN(c1ccccc1)c1ccc2c(c1)Oc1cc(OCc3ccc(OCC4=C(C(=O)O)N5C(=O)[C@H](NC(=O)Cc6ccccc6)[C@@H]5SC4)cc3)c(Br)cc1C21OC(=O)c2ccccc21] | (7S)-3-((4-(((2′-bromo-6′- (methyl(phenyl)amino)-3- oxo-3H- spiro[isobenzofuran-1,9′- xanthen]-3′- yl)oxy)methyl)phenoxy) methyl)-8-oxo-7-(2- phenylacetamido)- 5-thia-1- azabicyclo[4.2.0]oct-2- ene-2-carboxylic acid | Colorless to Red |
| MF-0015 | 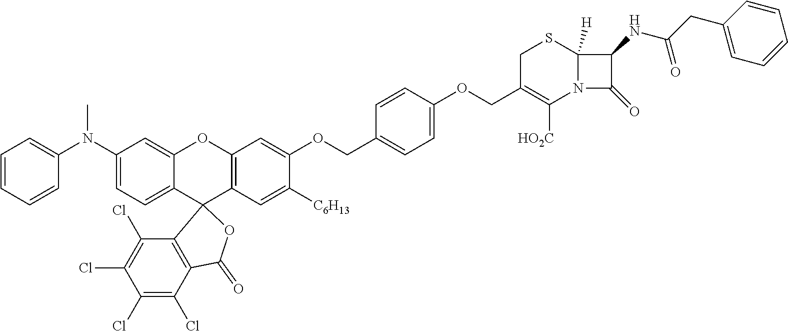 | (7S)-8-oxo-7-(2- phenylacetamido)-3-((4- (((4,5,6,7-tetrachloro-2′- hexyl-6′- (methyl(phenyl)amino)-3- oxo-3H- spiro[isobenzofuran-1,9′- xanthen]-3′- yl)oxy)methyl)phenoxy) methyl)-5-thia-1- azabicyclo[4.2.0]oct-2- ene-2-carboxylic acid | Colorless to Magenta |
| MF-0016 | 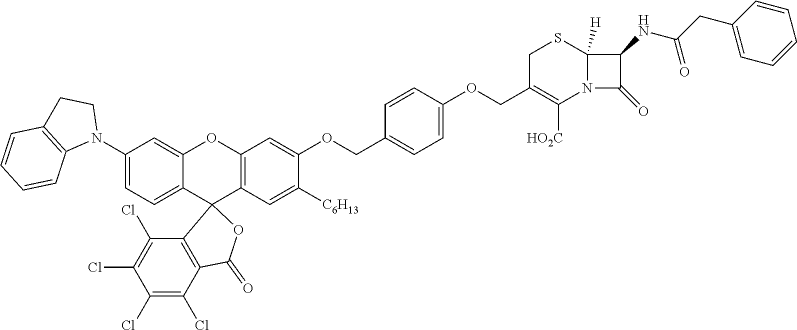 | (7S)-8-oxo-7-(2- phenylacetamido)-3-((4- (((4,5,6,7-tetrachloro-2′- hexyl-6′- (indolin-1-yl)-3- oxo-3H- spiro[isobenzofuran-1,9′- xanthen]-3′- yl)oxy)methyl)phenoxy) methyl)-5-thia-1- azabicyclo[4.2.0]oct-2- ene-2-carboxylic acid | Colorless to Violet |
| MF-0017 | 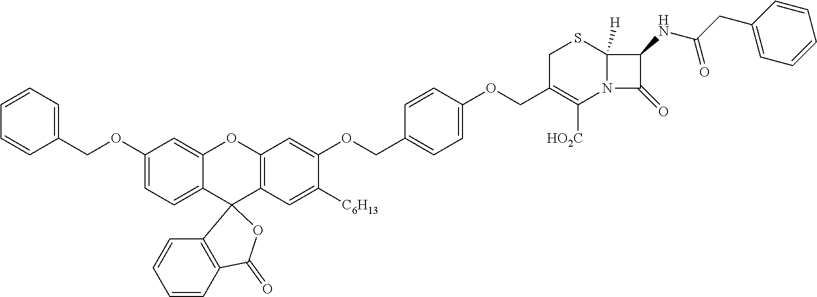 | (7S)-3-((4-(((6′- (benzyloxy)-2′-hexyl-3- oxo-3H- spiro[isobenzofuran-1,9′- xanthen]-3′- yl)oxy)methyl)phenoxy) methyl)-8-oxo-7-(2- phenylacetamido)-5-thia- 1-azabicyclo[4.2.0]oct-2- ene-2-carboxylic acid | Colorless to Yellow |
| MF-0018 | 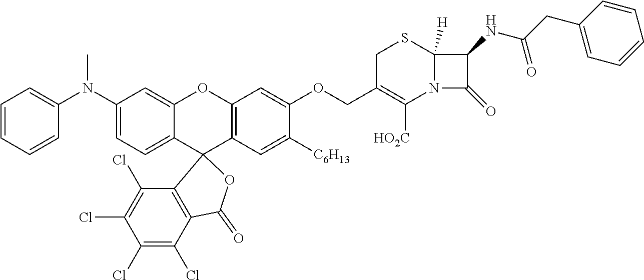 | (7S)-8-oxo-7-(2- phenylacetamido)-3-((4- (((4,5,6,7-tetrachloro-2′- hexyl-6′- (methyl(phenyl)amino)-3- oxo-3H- spiro[isobenzofuran-1,9′- xanthen]-3′- yl)oxy)methyl)-5-thia-1- azabicyclo[4.2.0]oct-2- ene-2-carboxylic acid | Colorless to Magenta |
| MF-0019 | 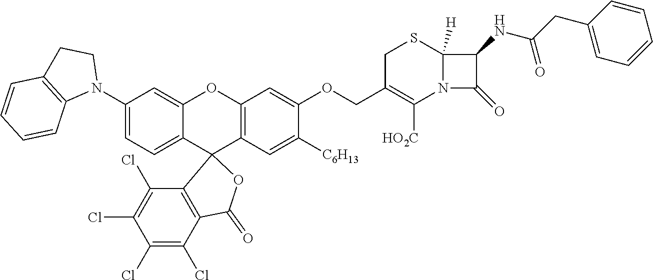 | (7S)-8-oxo-7-(2- phenylacetamido)-3-((4- (((4,5,6,7-tetrachloro-2′- hexyl-6′- (indolin-1-yl)-3- oxo-3H- spiro[isobenzofuran-1,9′- xanthen]-3′- yl)oxy)methyl)-5-thia-1- azabicyclo[4.2.0]oct-2- ene-2-carboxylic acid | Colorless to Violet |
| MF-0020 | 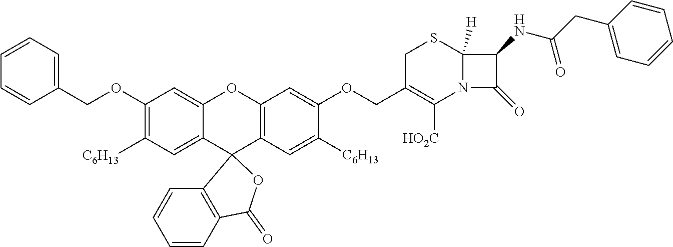 | (7S)-3-(((3′-(benzyloxy)- 2′,7′-dihexyl-3-oxo-3H- spirofisobenzofuran-1,9′- xanthen]-6′- yl)oxy)methyl)-8-oxo-7- (2-phenylacetamido)-5- thia-1- azabicyclo[4.2.0]oct-2- ene-2-carboxylic acid | Colorless to Yellow |
| MF-0025 | 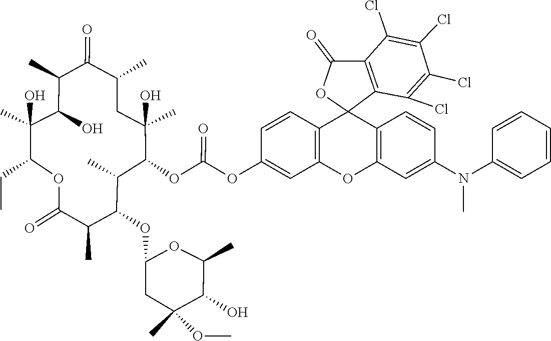 [SMILES: CC[C@H]1OC(=O)[C@H](C)[C@@H](O[C@H]2C[C@@](C)(OC)[C@@H](O)[C@H](C)O2)[C@H](C)[C@@H](OC(=O)Oc2ccc3c(c2)Oc2cc(N(C)c4ccccc4)ccc2C32OC(=O)c3c(Cl)c(Cl)c(Cl)c(Cl)c32)[C@](C)(O)C[C@@H](C)C(=O)[C@H](C)[C@@H](O)[C@]1(C)O] | (3R,4S,5S,6R,7R,9R,11R, 12R,13S,14R)-14-ethyl- 7,12,13-trihydroxy-4- (((2R,4R,5S,6S)-5- hydroxy-4-methoxy-4,6- dimethyltetrahydro-2H- pyran-2-yl)oxy)- 3,5,7,9,11,13-hexamethyl- 2,10-dioxo-1- oxacyclotetradecan-6-yl (4,5,6,7-tetrachloro-3′- (methyl(phenyl)amino)-3- oxo-3H- spiro[isobenzofuran-1,9′- xanthen]-6′-yl) carbonate | Colorless to Magenta |
| MF-0027 | 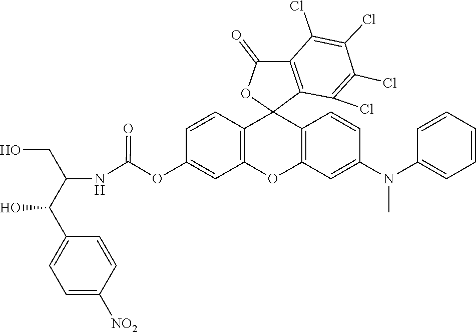 [SMILES: CN(c1ccccc1)c1ccc2c(c1)Oc1cc(OC(=O)NC(CO)[C@@H](O)c3ccc([N+](=O)[O-])cc3)ccc1C21OC(=O)c2c(Cl)c(Cl)c(Cl)c(Cl)c21] | 4,5,6,7-tetrachloro-3′- (methyl(phenyl)amino)-3- oxo-3H- spiro[isobenzofuran-1,9′- xanthen]-6′-yl ((1S)-1,3- dihydroxy-1-(4- nitrophenyl)propan-2- yl)carbamate | Colorless to Magenta |
| MF-0029 | 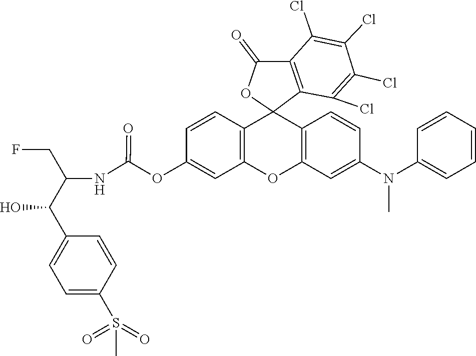 [SMILES: CN(c1ccccc1)c1ccc2c(c1)Oc1cc(OC(=O)NC(CF)[C@@H](O)c3ccc(S(C)(=O)=O)cc3)ccc1C21OC(=O)c2c(Cl)c(Cl)c(Cl)c(Cl)c21] | 4,5,6,7-tetrachloro-3′- (methyl(phenyl)amino)-3- oxo-3H- spirofisobenzofuran-1,9′- xanthen]-6′-yl ((1S)-3- fluoro-1-hydroxy-1-(4- (methylsulfonyl)phenyl) propan-2-yl)carbamate | Colorless to Magenta |
| MF-0050 | 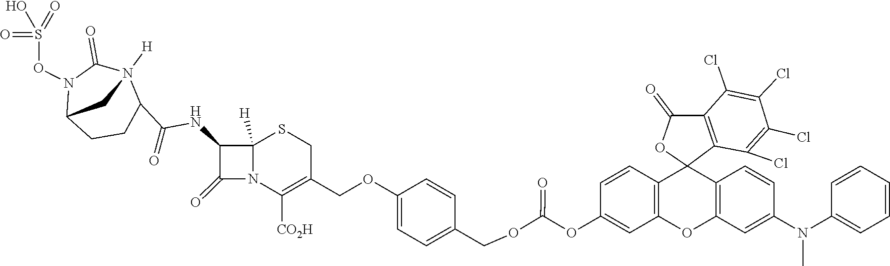 | (7R)-8-oxo-7-((1S,5S)-7- oxo-6-(sulfooxy)-114,6- diazabicyclo[3.2.1]octane- 2-carboxamido)-3-((4- (((((4,5,6,7-tetrachloro-3′- (methyl(phenyl)amino)-3- oxo-3H- spiro[isobenzofuran-1,9′- xanthen]-6′- yl)oxy)carbonyl)oxy)methyl) phenoxy)methyl)-5-thia-1- azabicyclo[4.2,0]oct-2- ene-2-carboxylic acid | Colorless to Magenta |
| MF-0051 | 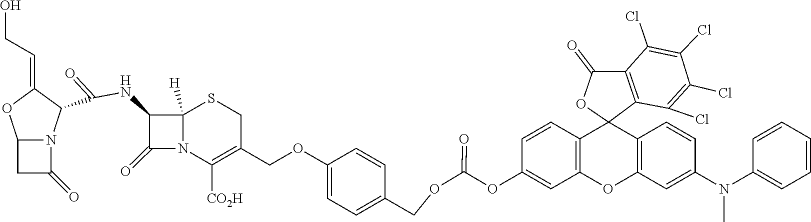 [SMILES: CN(c1ccccc1)c1ccc2c(c1)Oc1cc(OC(=O)OCc3ccc(OCC4=C(C(=O)O)N5C(=O)[C@@H](NC(=O)[C@H]6/C(=C/CO)OC7CC(=O)N76)[C@H]5SC4)cc3)ccc1C21OC(=O)c2c(Cl)c(Cl)c(Cl)c(Cl)c21] | (7R)-7-((2R,5R,Z)-3-(2- hydroxyethylidene)-7-oxo-4-oxa- 1-azabicyclo[3.2.0]heptane- 2-carboxamido)-8-oxo-3- ((4-(((((4,5,6,7-tetrachloro-3′- (methyl(phenyl)amino)-3- oxo-3H-spiro[isobenzofuran- 1,9′-xanthen]-6′- yl)oxy)carbonyl)oxy)methyl) phenoxy)methyl)-5- thia-1- azabicyclo[4.2,0]oct-2- ene-2-carboxylic acid | Colorless to Magenta |
| MF-0052 | 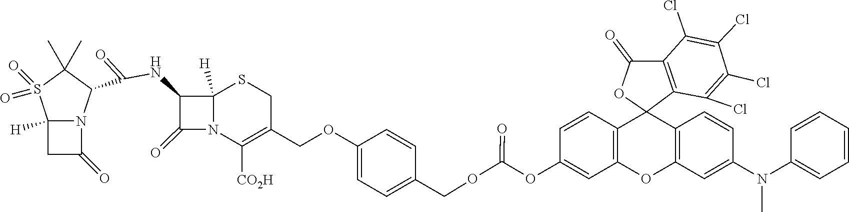 [SMILES: CN(c1ccccc1)c1ccc2c(c1)Oc1cc(OC(=O)OCc3ccc(OCC4=C(C(=O)O)N5C(=O)[C@@H](NC(=O)[C@@H]6N7C(=O)C[C@H]7S(=O)(=O)C6(C)C)[C@H]5SC4)cc3)ccc1C21OC(=O)c2c(Cl)c(Cl)c(Cl)c(Cl)c21] | (7R)-7-((2S,5R)-3,3-dimethyl- 4,4-dioxido-7-oxo-4-thia-1- azabicyclo[3.2.0]heptane- 2-carboxamido)-8-oxo-3- ((4-(((((4,5,6,7- tetrachloro-3′- (methyl(phenyl)amino)-3- oxo-3H-spiro[isobenzofuran- 1,9′-xanthen]-6′- yl)oxy)carbonyl)oxy)methyl) phenoxy)methyl)-5- thia-1-azabicyclo[4.2.0]oct-2- ene-2-carboxylic acid | Colorless to Magenta |
| MF-0053 | 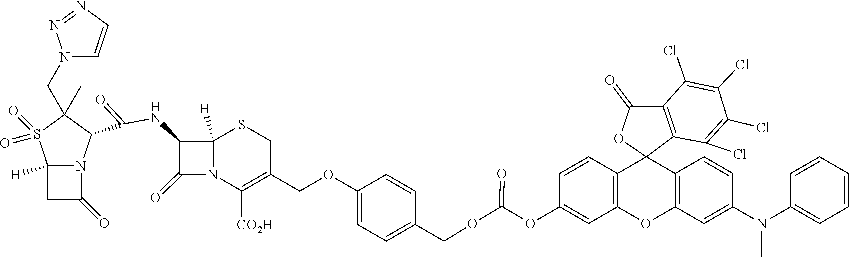 [SMILES: CN(c1ccccc1)c1ccc2c(c1)Oc1cc(OC(=O)OCc3ccc(OCC4=C(C(=O)O)N5C(=O)[C@@H](NC(=O)[C@@H]6N7C(=O)C[C@H]7S(=O)(=O)C6(C)Cn6ccnn6)[C@H]5SC4)cc3)ccc1C21OC(=O)c2c(Cl)c(Cl)c(Cl)c(Cl)c21] | (7R)-7-((2S,3R,5R)-3- ((1H-1,2,3-triazol-1- yl)mcthyl)-3-methyl-4,4- dioxido-7-oxo-4-thia-- azabicyclo[3.2.0]heptane- 2-carboxamido)-8-oxo-3- ((4-(((((4,5,6,7- tetrachloro-3′- (methyl(phenyl)amino)-3- oxo-3H- spiro[isobenzofuran-1,9′- xanthen]-6′- yl)oxy)carbonyl)oxy)methyl) phenoxy)methyl)-5- thia-1-azabicyclo[4.2,0]oct-2- ene-2-carboxylic acid | Colorless to Magenta |
| MF-0054 | 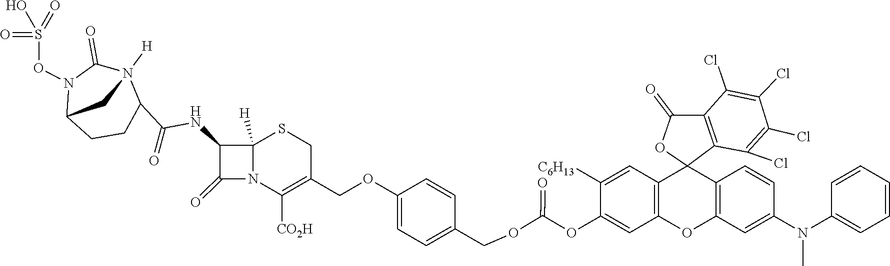 | (7R)-8-oxo-7-((1S,5S)-7- oxo-6-(sulfooxy)-114,6- diazabicyclo[3.2.1]octane- 2-carboxamido)-3-((4- (((((4,5,6,7-tetrachloro-2′- hexyl-6′-(indolin-1-yl)-3- oxo-3H- spiro[isobenzofuran-1,9′- xanthen]-3′- yl)oxy)carbonyl)oxy)methyl) phenoxy)methyl)-5- thia-1- azabicyclo[4.2.0]oct-2- ene-2-carboxylic acid | Colorless to violet |
| MF-0055 | 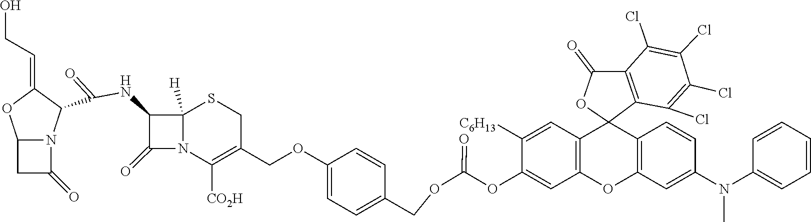 | (7R)-7-((2R,5R,Z)-3-(2- hydroxyethylidene)-7-oxo- 4-oxa-1-azabicyclo[3,2.0] heptane-2-carboxamido)-8- oxo-3-((4-(((((4,5,6,7- tetrachloro-2′-hexyl-6′- (indolin-1-yl)-3-oxo-3H- spiro[isobenzofuran-1,9′- xanthen]-3′- yl)oxy)carbonyl)oxy)methyl) phenoxy)methyl)-5- thia-1-azabicyclo[4.2.0]oct-2- ene-2-carboxylic acid | Colorless to violet |
| MF-0056 | 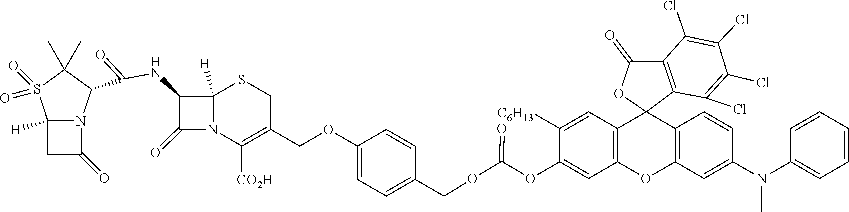 | (7R)-7-((2S,5R)-3,3-dimethyl- 4,4-dioxido-7-oxo-4-thia-1- azabicyclo[3.2.0]heptane- 2-carboxamido)-8-oxo-3- (4-(((((4,5,6,7-tetrachloro-2′- hexyl-6′-(indolin-1-yl)-3-oxo- 3H-spiro[isobenzofuran-1,9′- xanthen]-3′- yl)oxy)carbonyl)oxy)methyl) phenoxy)methyl)-5- thia-1- azabicyclo[4.2.0]oct-2- ene-2-carboxylic acid | Colorless to violet |
| MF-0057 | 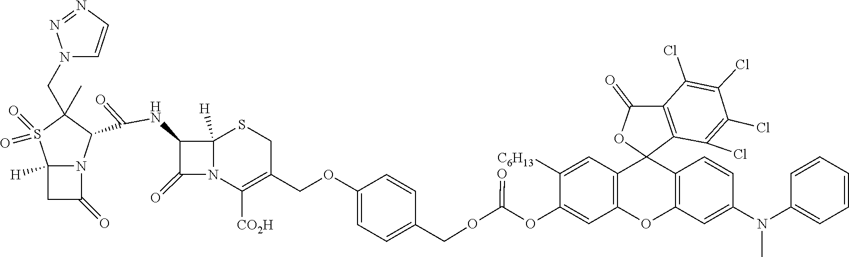 | (7R)-7-((2S,5R)-3- ((1H-1,2,3-triazol-1-yl) methyl)-3-methyl-4,4- dioxido-7-oxo-4-thia-1- azabicyclo[3.2.0]heptane- 2-carboxamido)-8-oxo-3- ((4-(((((4,5,6,7-tetrachloro- 2′-hexyl-6′-(indolin-1-yl)-3- oxo-3H-spiro[isobenzofuran- 1,9′-xanthen]-3′-yl)oxy)carbonyl) oxy)methyl)phenoxy)methyl)- 5-thia-1-azabicyclo[4.2.0]oct-2- ene-2-carboxylic acid | Colorless to violet |

In an embodiment, the biosensor is a compound derived from dithiofluorescein and cephalosporin represented by Formula (12)

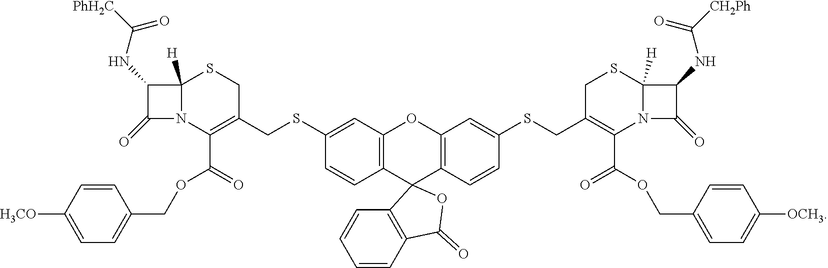

In some embodiments, the biosensor is a compound selected from those disclosed in Table 3 that comprise Formula (11).

TABLE 3
Embodiments of biosensors derived from 2-nitroaniline.

| Entry | | | Optical |
|---|---|---|---|
| No. | Compound | Chemical Name | Response |
| MF-0058 | 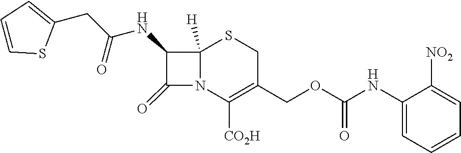 [SMILES: O=C(Cc1cccs1)N[C@@H]1C(=O)N2C(C(=O)O)=C(COC(=O)Nc3ccccc3[N+](=O)[O-])CS[C@H]12] | (6R,7R)-3-((((2- nitrophenyl)carbamoyl) oxy)methyl)-8-oxo-7- (2-(thiophen-2- yl)acetamido)-5-thia-1- azabicyclo[4.2.0]oct-2- ene-2-carboxylic acid | Colorless to Yello |
| MF-0059 | 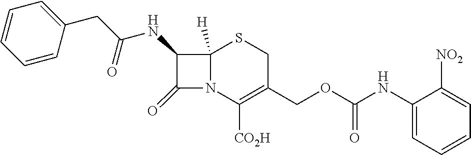 [SMILES: O=C(Cc1ccccc1)N[C@@H]1C(=O)N2C(C(=O)O)=C(COC(=O)Nc3ccccc3[N+](=O)[O-])CS[C@H]12] | (6R,7R)-3-((((2- nitrophenyl)carbamoyl) oxy)methyl)-8-oxo-7-(2- phenylacetamido)-5- thia-1- azabicyclo[4.2.0]oct-2- ene-2-carboxylic acid | Colorless to Yellow |

In some embodiments, the biosensor is a compound selected from those disclosed in Table 1.

TABLE 1
Embodiments of biosensors derived from Methylene Blue

| | | | Microbe |
|---|---|---|---|
| | | | inactivating |
| Entry | Compound | Chemical Name | factor |
| MF- 0060 | 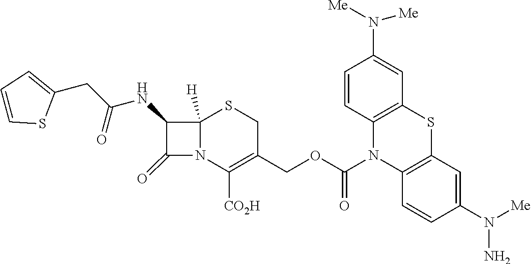 [SMILES: CN(C)c1ccc2c(c1)Sc1cc(N(C)N)ccc1N2C(=O)OCC1=C(C(=O)O)N2C(=O)[C@@H](NC(=O)Cc3cccs3)[C@H]2SC1] | (6R,7R)-3- (((3,7-bis (dimethyl- amino)- 10H- phenothiazine- 10- carbonyl)oxy) methyl)-8-oxo- 7-(2-(thiophen- 2-yl) acetamido)- 5-thia-1- azabicyclo[4.2.0] oct-2-ene-2- carboxylic acid | Bacteria lactamase |
| MF- 0061 | 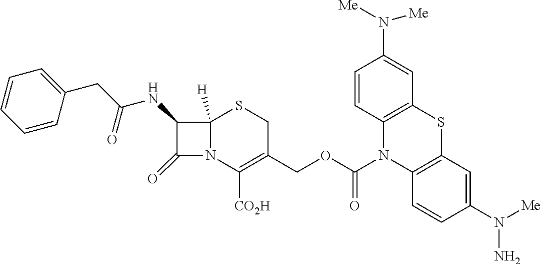 [SMILES: CN(C)c1ccc2c(c1)Sc1cc(N(C)N)ccc1N2C(=O)OCC1=C(C(=O)O)N2C(=O)[C@@H](NC(=O)Cc3ccccc3)[C@H]2SC1] | 6R,7R)-3- (((3,7-bis (dimethyl- amino)- 10H- phenothiazine- 10- carbonyl)oxy) methyl)-8-oxo- 7-(2-phenyl- acetamido)- 5-thia-1- azabicyclo [4.2.0] oct-2-ene-2- carboxylic acid | Bacteria lactamase |
| MF- 0062 | 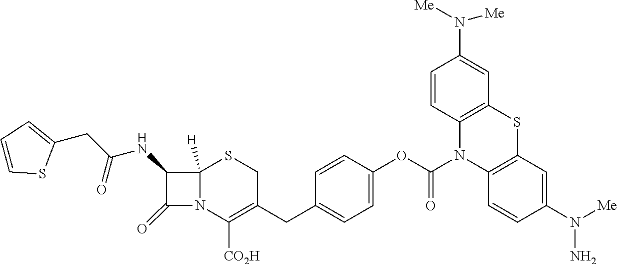 [SMILES: CN(C)c1ccc2c(c1)Sc1cc(N(C)N)ccc1N2C(=O)Oc1ccc(CC2=C(C(=O)O)N3C(=O)[C@@H](NC(=O)Cc4cccs4)[C@H]3SC2)cc1] | (6R,7R)-3- (((3,7-bis (dimethyl- amino)-10H- phenothiazine- 10- carbonyl)oxy) benzyl)-8-oxo- 7-(2-(thiophen- 2-yl) acetamido)- 5-thia-1- azabicyclo [4.2.0] oct-2-ene-2- carboxylic acid | Bacteria lactamase |
| MF- 0063 | 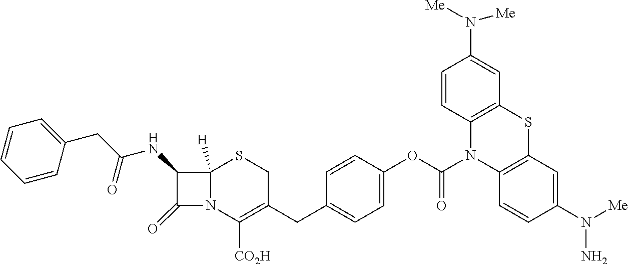 [SMILES: CN(C)c1ccc2c(c1)Sc1cc(N(C)N)ccc1N2C(=O)Oc1ccc(CC2=C(C(=O)O)N3C(=O)[C@@H](NC(=O)Cc4ccccc4)[C@H]3SC2)cc1] | (6R,7R)-3- (((3,7-bis (dimethyl- amino)- 10H- phenothiazine- 10- carbonyl)oxy) benzyl)-8-oxo- 7-(2-(phenyl- acetamido)- 5-thia-1- azabicyclo [4.2.0] oct-2-ene-2- carboxylic acid | Bacteria lactamase |
| MF- 0064 | 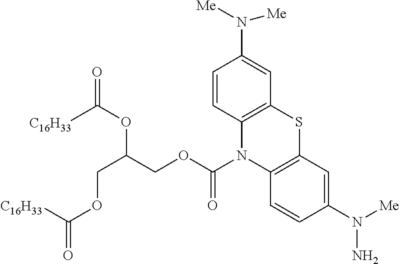 | 3-((3,7-bis (dimethyl- amino)- 10H- phenothiazine- 10- carbonyl)oxy) propane-1,2- diyl dihepta- decanoate | Candida albicans lipase |
| MF- 0065 | 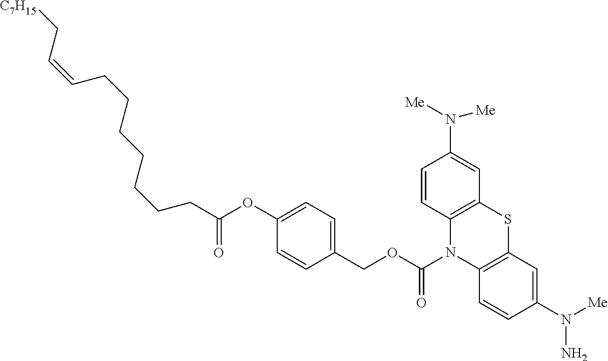 | (Z)-4- (oleoyloxy) benzyl 3,7-bis (dimethyl- amino)- 10H- pheno- thiazine- 10- carboxylate | Microbial esterase-lipase |
| MF- 0066 | 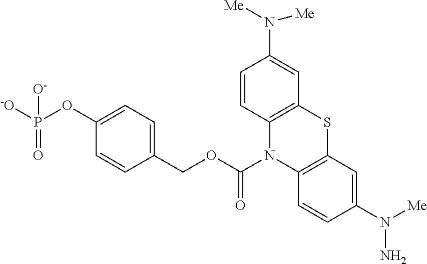 | 4-(((3,7-bis (dimethy- lamino)- 10H- pheno- thiazine- 10- carbonyl) oxy)methyl) phenyl phosphate | phosphatase |
| MF- 0067 | 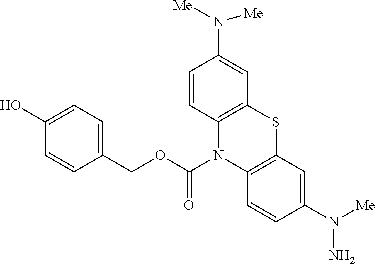 [SMILES: CN(C)c1ccc2c(c1)Sc1cc(N(C)N)ccc1N2C(=O)OCc1ccc(O)cc1] | 4- hydroxy- phenethyl 3,7-bis (dimethyl- amino)- 10H- pheno- thiazine- 10- carboxylate | tyrosinase |
| MF- 0068 | 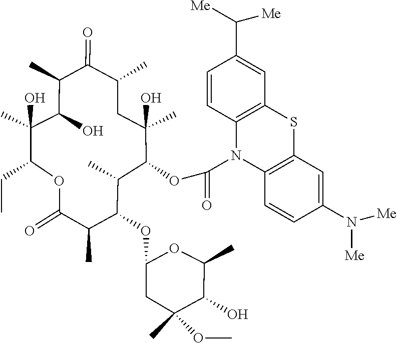 [SMILES: CC[C@H]1OC(=O)[C@H](C)[C@@H](O[C@H]2C[C@@](C)(OC)[C@@H](O)[C@H](C)O2)[C@H](C)[C@@H](OC(=O)N2c3ccc(C(C)C)cc3Sc3cc(N(C)C)ccc32)[C@](C)(O)C[C@@H](C)C(=O)[C@H](C)[C@@H](O)[C@]1(C)O] | (3R,4S,5S,6R, 7R,9R,11R,12R, 13S,14R)-14- ethyl-7,12,13- trihydroxy-4- (((2R,4R,5S, 6S)-5-hydroxy- 4-methoxy- 4,6-dimethyl- tetrahydro-2H- pyran-2-yl)oxy)- 3,5,7,9,11,13- hexamethyl- 2,10-dioxooxacy- clotetradecan- 6-yl 3,7-bis (dimethyl- amino)-10H- phenothiazine- 10-carboxylate | Esterase secreted by Erythromycin resistant bacteria |
| MF- 0069 | 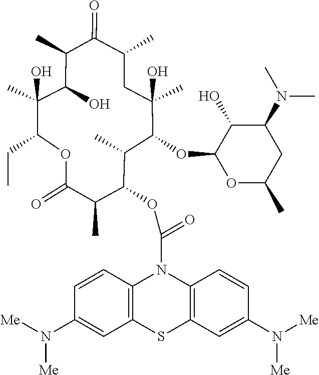 [SMILES: CC[C@H]1OC(=O)[C@H](C)[C@@H](OC(=O)N2c3ccc(N(C)C)cc3Sc3cc(N(C)C)ccc32)[C@H](C)[C@@H](O[C@@H]2O[C@H](C)C[C@H](N(C)C)[C@H]2O)[C@](C)(O)C[C@@H](C)C(=O)[C@H](C)[C@@H](O)[C@]1(C)O] | (3R,4S,5S,6R, 7R,9R,11R, 12R,13S,14R)- 6-(((2S,3R, 4S,6R)-4- (dimethyl- amino)-3- hydroxy-6- methyl- tetrahydro-2H- pyran-2-yl)oxy)- 14-ethyl-7,12,13- trihydroxy- 3,5,7,9,11,13- hexamethyl- 2,10-dioxooxacy- clotetradecan- 4-yl 3,7-bis (dimethy- lamino)-10H- phenothiazine- 10-carboxylate | Esterase secreted by Erythromycin resistant bacteria |
| MF- 0070 | 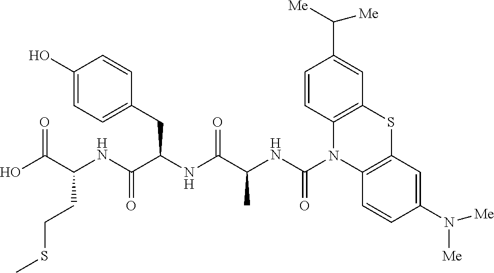 [SMILES: CSCC[C@@H](NC(=O)[C@@H](Cc1ccc(O)cc1)NC(=O)[C@H](C)NC(=O)N1c2ccc(C(C)C)cc2Sc2cc(N(C)C)ccc21)C(=O)O] | (3,7-bis (dimethyl- amino)- 10H- phenothiazine- 10- carbonyl)- alanyl-tyrosyl- methionine | peptidase |
| MF- 0071 | 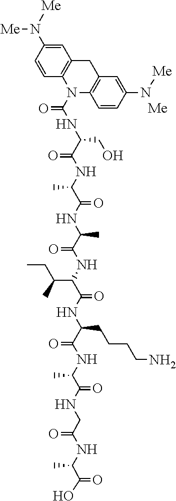 | (3,7-bis (dimethyl- amino)- 10H- phenothiazine- 10- carbonyl)- seryl-alanyl- alanyl- isoleucyl- lysyl- alanyl-glycyl- alanine | Streptococcus pyogenes SpeC |
| MF- 0072 | 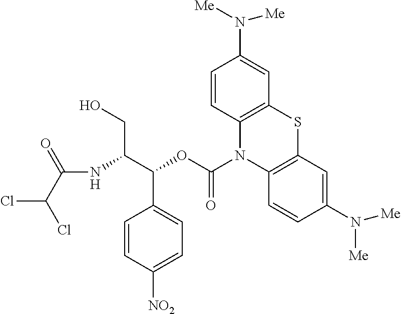 [SMILES: CN(C)c1ccc2c(c1)Sc1cc(N(C)C)ccc1N2C(=O)O[C@H](c1ccc([N+](=O)[O-])cc1)[C@@H](CO)NC(=O)C(Cl)Cl] | (1R,2R)- 2-(2,2- dichioro- acetamido)-3- hydroxy-1-(4- nitrophenyl) propyl 3,7-bis (dimethyl- amino)-10H- phenothiazine- 10- carboxylate | Cloramphenicol antibiotic resistant organisms |
| MF- 0073 | 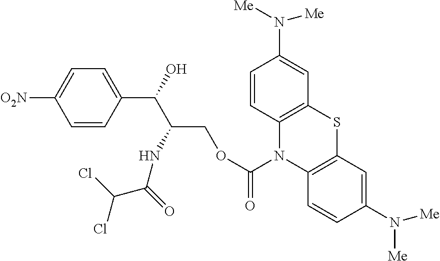 [SMILES: CN(C)c1ccc2c(c1)Sc1cc(N(C)C)ccc1N2C(=O)OC[C@H](NC(=O)C(Cl)Cl)[C@@H](O)c1ccc([N+](=O)[O-])cc1] | (2S,3S)-2- (2,2- dichioro- acetamido)-3- hydroxy-3-(4- nitrophenyl) propyl 3,7-bis (dimethyl- amino)-10H- phenothiazine- 10- carboxylate | Cloramphenicol antibiotic resistant organisms |
| MF- 0074 | 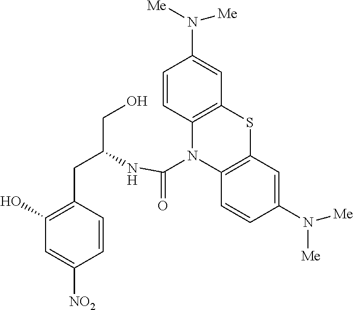 | N-((1S,2S)- 1,3-dihydroxy- 1-(4- nitrophenyl) propan-2- yl)-3,7-bis (dimethy- lamino)- 10H- phenothiazine- 10- carboxamide | Cloramphenicol antibiotic resistant organisms |
| MF- 0075 | 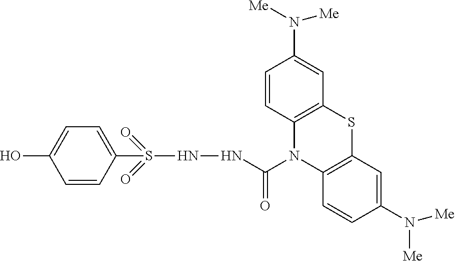 [SMILES: CN(C)c1ccc2c(c1)Sc1cc(N(C)C)ccc1N2C(=O)NNS(=O)(=O)c1ccc(O)cc1] | N′-(3,7-bis (dimethy- lamino)- 10H- phenothiazine- 10-carbonyl)- 4-hydroxy- benzene- sulfono- hydrazide | Oxidizing environment |
| MF- 0076 | 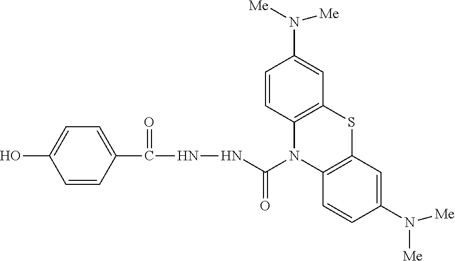 [SMILES: CN(C)c1ccc2c(c1)Sc1cc(N(C)C)ccc1N2C(=O)NNC(=O)c1ccc(O)cc1] | 3,7-bis (dimethyl- amino)-N′- (4- hydroxy- benzoyl)- 10H- phenothiazine- 10- carbohydrazide | Oxidizing environment |
| MF- 0077 | 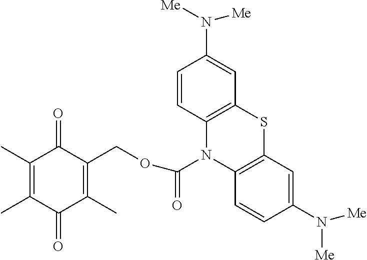 [SMILES: CC1=C(C)C(=O)C(COC(=O)N2c3ccc(N(C)C)cc3Sc3cc(N(C)C)ccc32)=C(C)C1=O] | (2,4,5- trimethyl-3,6- dioxocy- clohexa- 1,4-dien-l-yl) methyl 3,7-bis (dimethyl- amino)-10H- phenothiazine- 10- carboxylate | glutathione |
| MF- 0078 | 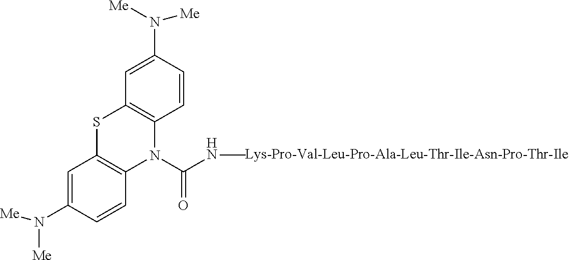 [SMILES: CNC(=O)N1c2ccc(N(C)C)cc2Sc2cc(N(C)C)ccc21] | (3,7-bis (dimethyi- amino)-10H- phenothiazine- 10-carbonyl)- methionyl- leucyl-alanyl- arginyl-arginyl- lysyl-prolyl- valyl-leucyl- prolyl-alanyl- leucyl-threonyl- isoleucyl- asparaginyl- prolyl-threonyl- isoleucine | Bacillus anthracis lethal factor |
| MF- 0079 | 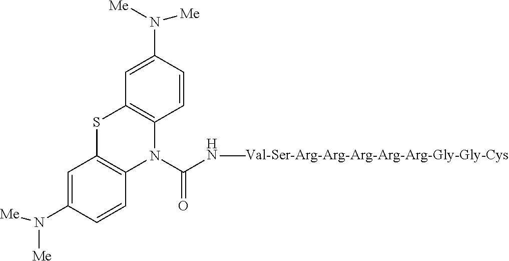 [SMILES: CNC(=O)N1c2ccc(N(C)C)cc2Sc2cc(N(C)C)ccc21] | (3,7-bis (dimethyl- amino)-10H- phenothiazine- 10-carbonyl)- valyl-seryl- arginyl- arginyl- arginyl- arginyl- arginyl- glycyl- glycyl- cysteine | E coli, Omp T |
| MF- 0080 | 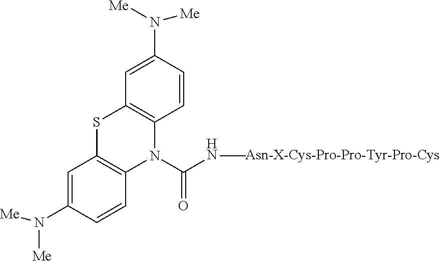 [SMILES: CNC(=O)N1c2ccc(N(C)C)cc2Sc2cc(N(C)C)ccc21] | (3,7-bis (dimethyl- amino)-10H- phenothiazine- 10-carbonyl)- carbonyl)- asparaginyl- X-cysteinyl- prolyl-prolyl- tyrosyl- prolyl- cysteine | Streptococcus pneumonia, IgA specific serine endopeptidase |
| MF- 0081 | 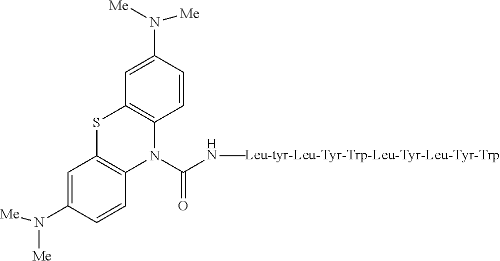 [SMILES: CNC(=O)N1c2ccc(N(C)C)cc2Sc2cc(N(C)C)ccc21] | (3,7-bis (dimethyl- amino)-10H- phenothiazine- 10-carbonyl)- leucyl-tyrosyl- leucyl-tyrosyl- trptophyl- leucyl-tyrosyl- leucyl-tyrosyl- trptophan | Streptococcus pneumonia, ClpP |
| MF- 0082 | 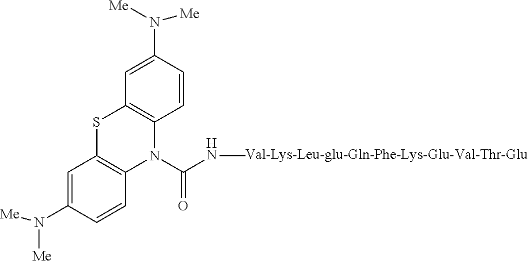 [SMILES: CNC(=O)N1c2ccc(N(C)C)cc2Sc2cc(N(C)C)ccc21] | (3,7-bis (dimethyl- amino)-10H- phenothiazine- 10-carbonyl)- valyl-lysyl- leucyl- glutamyl- glutaminyl- phenylalanyl- lysyl-glutamyl- valyl- threonyl- glutamic acid | Streptococcus pneumonia, HtrA |
| MF- 0083 | 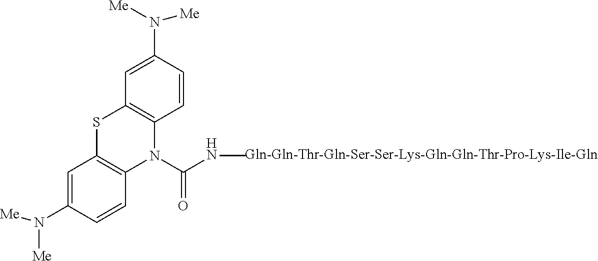 [SMILES: CNC(=O)N1c2ccc(N(C)C)cc2Sc2cc(N(C)C)ccc21] | (3,7-bis (dimethyl- amino)-10H- phenothiazine- 10-carbonyl)- glutaminyl- glutaminyl- threonyl- glutaminyl- seryl-seryl- lysyl- glutaminyl- glutaminyl- threonyl- prolyl-lysyl- isoleucyl- | Staphylococcus aureus SspA |
| | | glutamine | |
| MF- 0084 | 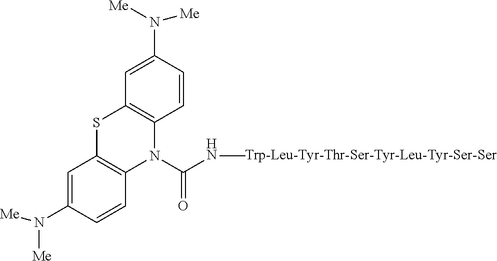 [SMILES: CNC(=O)N1c2ccc(N(C)C)cc2Sc2cc(N(C)C)ccc21] | (3,7-bis (dimethyl- amino)-10H- phenothiazine- 10-carbonyl)- tryptophyl- leucyl-tyrosyl- threonyl- seryl-tyrosyl- leucyl- tyrosyl- seryl-serine | Staphylococcus aureus Spls |
| MF- 0021 | 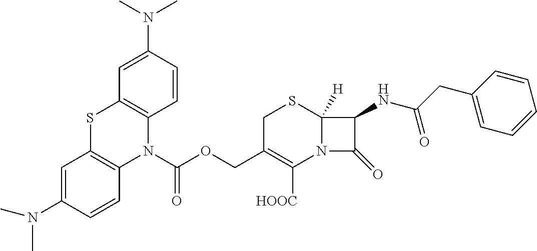 [SMILES: CN(C)c1ccc2c(c1)Sc1cc(N(C)C)ccc1N2C(=O)OCC1=C(C(=O)O)N2C(=O)[C@H](NC(=O)Cc3ccccc3)[C@@H]2SC1] | (7S)-3-(((3,7- bis(dimethyl- amino)-10H- phenothiazine- 10-carbonyl) oxy)methyl)-8- oxo-7-(2- phenyl- acetamido)- 5-thia-1- azabicyclo [4.2.0]oct-2- ene-2- carboxylic acid | Colorless to Blue |
| MF- 0022 | 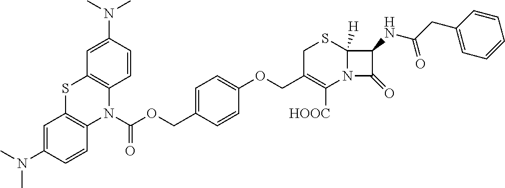 [SMILES: CN(C)c1ccc2c(c1)Sc1cc(N(C)C)ccc1N2C(=O)OCc1ccc(OCC2=C(C(=O)O)N3C(=O)[C@H](NC(=O)Cc4ccccc4)[C@@H]3SC2)cc1] | (7S)-3-((4- (((3,7-bis (dimethyl- amino)-10H- phenothiazine- 10-carbonyl) oxy)methyl) phenoxy) methyl)-8-oxo-7- (2-phenyl- acetamido)-5- thia-1- | Colorless to Blue |
| | | azabicyclo[4.2.0] | |
| | | oct-2-ene-2- | |
| | | carboxylic acid | |
| MF- 0023 | 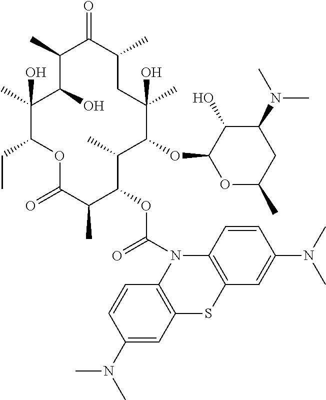 [SMILES: CC[C@H]1OC(=O)[C@H](C)[C@@H](OC(=O)N2c3ccc(N(C)C)cc3Sc3cc(N(C)C)ccc32)[C@H](C)[C@@H](O[C@@H]2O[C@H](C)C[C@H](N(C)C)[C@H]2O)[C@](C)(O)C[C@@H](C)C(=O)[C@H](C)[C@@H](O)[C@]1(C)O] | (3R,4S,5S, 6R,7R,9R,11R, 12R,13S,14R)- 6-(((2S,3R,4S, 6R)-4- (dimethyl- amino)-3- hydroxy-6- methyl- tetrahydro-2H- pyran-2-yl)oxy)- 14-ethyl- 7,12,13- trihydroxy- 3,5,7,9,11,13- hexamethyl-2,10- dioxooxacy- clotetradecan- 4-yl 3,7- bis(dimethyl- amino)-10H- phenothiazine- 10-carboxylate | Colorless to Blue |
| MF- 0024 | 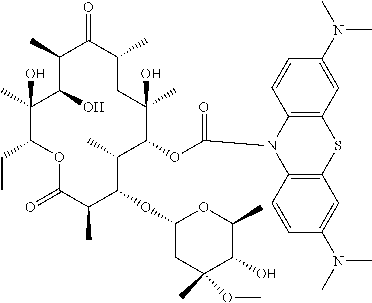 [SMILES: CC[C@H]1OC(=O)[C@H](C)[C@@H](O[C@H]2C[C@@](C)(OC)[C@@H](O)[C@H](C)O2)[C@H](C)[C@@H](OC(=O)N2c3ccc(N(C)C)cc3Sc3cc(N(C)C)ccc32)[C@](C)(O)C[C@@H](C)C(=O)[C@H](C)[C@@H](O)[C@]1(C)O] | (3R,4S,5S,6R,7R, 9R,11R,12R,13S, 14R)-14-ethyl- 7,12,13- trihydroxy-4- (((2R,4R,5S,6S)- 5-hydroxy-4- methoxy-4,6- dimethyl- tetrahydro-2H- pyran-2-yl)oxy)- 3,5,7,9,11,13- hexamethyl-2,10- dioxooxacy- clotetradecan-6-yl 3,7-bis(dimethy- lamino)-10H- phenothiazine- 10-carboxylate | Colorless to Blue |
| MF- 0026 | 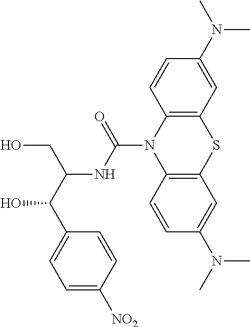 [SMILES: CN(C)c1ccc2c(c1)Sc1cc(N(C)C)ccc1N2C(=O)NC(CO)[C@@H](O)c1ccc([N+](=O)[O-])cc1] | N-((1S)-1,3- dihydroxy-1- (4-nitrophenyl) propan-2- yl)-3,7- bis(dimethyl- amino)-10H- phenothiazine- 10- carboxamide | Colorless to Blue |
| MF- 0028 | 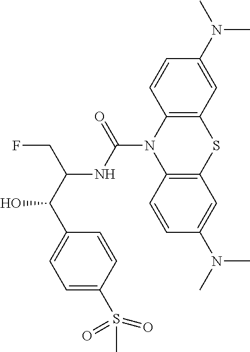 [SMILES: CN(C)c1ccc2c(c1)Sc1cc(N(C)C)ccc1N2C(=O)NC(CF)[C@@H](O)c1ccc(S(C)(=O)=O)cc1] | 3,7-bis (dimethyl- amino)-N- ((1S)-3- fluoro-1- hydroxy- l-(4- (methylsulfonyl) phenyl)propan- 2-yl)-10H- phenothiazine- 10- carboxamide | Colorless to Blue |
| MF- 0046 | 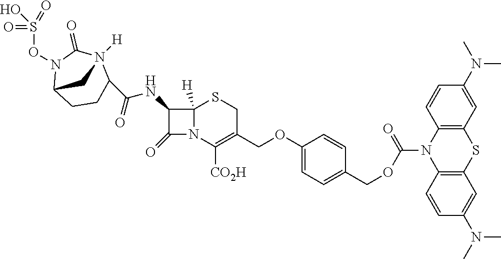 | (7R)-3-((4-(((3,7- bis(dimethyl- amino)-10H- phenothiazine- 10-carbonyl)oxy) methyl)phenoxy) methyl)-8-oxo-7- ((1S,5S)-7-oxo- 6-(sulfooxy)- 114,6- diazabicyclo [3.2.l]octane-2- carboxamido)- 5-thia-1- azabicyclo [4.2.0]oct-2-ene- | Colorless to Blue |
| | | 2-carboxylic acid | |
| MF- 0047 | 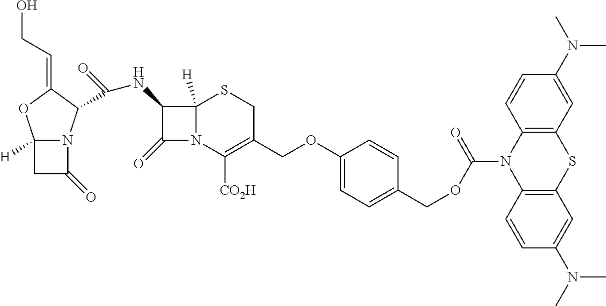 [SMILES: CN(C)c1ccc2c(c1)Sc1cc(N(C)C)ccc1N2C(=O)OCc1ccc(OCC2=C(C(=O)O)N3C(=O)[C@@H](NC(=O)[C@H]4/C(=C/CO)O[C@@H]5CC(=O)N54)[C@H]3SC2)cc1] | (7R)-3-((4-(((3,7- bis(dimethyl- amino)-10H- phenothiazine- 10-carbonyl) oxy)methyl) phenoxy) methyl)- 7-((2R,5R,Z)- 3-(2-hydroxy- ethylidene)-7- oxo-4-oxa-1- azabicyclo[3,2.0] heptane-2- carboxamido)-8- oxo-5-thia-1- azabicyclo[4.2.0] oct-2-ene-2- carboxylic acid | Colorless to Blue |
| MF- 0048 | 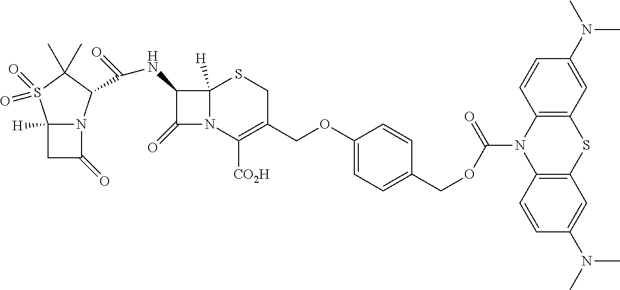 [SMILES: CN(C)c1ccc2c(c1)Sc1cc(N(C)C)ccc1N2C(=O)OCc1ccc(OCC2=C(C(=O)O)N3C(=O)[C@@H](NC(=O)[C@@H]4N5C(=O)C[C@H]5S(=O)(=O)C4(C)C)[C@H]3SC2)cc1] | (7R)-3-((4-(((3,7- bis(dimethyl- amino)-10H- phenothiazine- 10-carbonyl)oxy) methyl)phenoxy) methyl)-7- ((2S,5R)-3,3- dimethyl-4,4- dioxido- 7-oxo-4-thia-l- azabicyclo[3,2.0] heptane-2- carboxamido)- 8-oxo-5-thia-1- azabicyclo[4.2.0] oct-2-ene-2- carboxylic acid | Colorless to Blue |
| MF- 0049 | 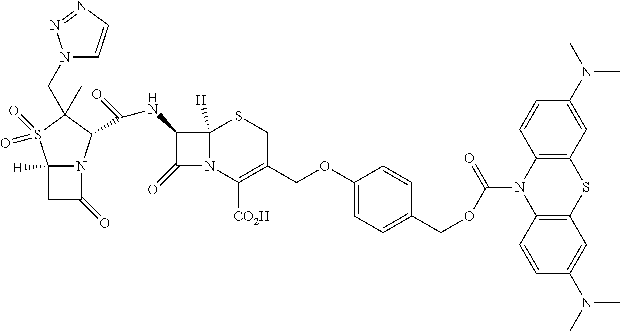 [SMILES: CN(C)c1ccc2c(c1)Sc1cc(N(C)C)ccc1N2C(=O)OCc1ccc(OCC2=C(C(=O)O)N3C(=O)[C@@H](NC(=O)[C@@H]4N5C(=O)C[C@H]5S(=O)(=O)C4(C)Cn4ccnn4)[C@H]3SC2)cc1] | (7R)-7- ((2S,3R,5R)-3- ((1H-1,2,3-triazol- 1-yl)methyl)-3- methyl-4,4- dioxido-7-oxo- 4-thia-1- azabicyclo[3.2.0] heptane-2- carboxamido)-3- ((4-(((3,7-bis (dimethyl- amino)-10H- phenothiazine- 10-carbonyl) oxy)methyl) phenoxy)methyl)- 8-oxo-5-thia-1- azabicyclo[4.2.0] oct-2-ene- | Colorless to Blue |
| | | 2-carboxylic acid | |

In one aspect this disclosure provides novel rationally designed molecule libraries (e.g. provided in Table 1) that can detect specific microbes. Using standard screening tools these molecules can be evaluated for their ability to selectively and specifically detect microbes and even destroy them.

In some embodiments the screening library is based on a platform containing other leuco dyes including but not limited to, spiropyrans, quinones, thiazines, phenazines, oxazines, pthalide-type dyes, triarylmethanes, fluorans, and tetrazoliums.

In some embodiments the screening library is based on a platform containing naturally occurring dyes including but not limited to, curcumins, hypericin, carotenes, anthocynanins, and any other phytochemical dyes.

In some embodiments the screening library is based on a platform containing synthetic dyes that may not be leuco dyes for e.g. azo dyes, Xanthenes, phthalides and azomethine dyes.

Screening such libraries will enable us to identify one or more molecules that can be used as a biosensor for sensitive and specific detection of certain microbes. These molecules may also have the ability to destroy the microbes making them multifunctional.

### Device and Composition Containing Biosensor

In some embodiments, the biosensors are prepared into various product forms that are used to sequester and/or identify bacteria (e.g., Gram-positive bacteria and/or Gram-negative bacteria), screen liquid samples (e.g., water samples, food samples, pharmaceutical samples, blood samples, blood platelet samples, etc.) for the presence of bacteria (e.g., Gram-positive bacteria and/or Gram-negative bacteria), treat disorders associated with bacteria (e.g., Gram-positive bacteria and/or Gram-negative bacteria), and/or identify, sequester, and remove bacteria (e.g., Gram-positive bacteria and/or Gram-negative bacteria) from a liquid sample (e.g., water sample, a wound site or a wound closure device or wound dressing.

In some embodiments, the biosensor is formulated as a diagnostic composition for detecting drug-resistant bacteria. In some embodiments, the biosensor is formulated as a pharmaceutical composition comprising the biosensor compound of formulae (1)-(5) and (17-26) and a pharmaceutically acceptable excipient. In some embodiments, the biosensor is covalently conjugated to a dendritic polymer support to form a dendrimer biosensor. In some embodiments, the biosensor is formulated as a diagnostic particle. In some embodiments, the biosensor is formulated as a theragnostic particle.

### Biosensor Pharmaceutical Composition

In some embodiments, the disclosure provides a pharmaceutical composition for administering a biosensor to an infection site in a subject. In some embodiments, the pharmaceutical composition comprises the biosensor disclosed herein and a pharmaceutically acceptable excipient typically in the form of gel, powder, creams, lotion, ointment, emulsion, or liquid formulation. In some embodiments, the pharmaceutical formulation may additionally comprise a pharmaceutically acceptable excipient including solvents, dispersion media, diluents, or other liquid vehicles, dispersion or suspension aids, surface active agents, isotonic agents, thickening or emulsifying agents, preservatives, solid binders, lubricants, antimicrobial preservatives, antioxidants and other excipients such as dispersing, suspending, thickening, emulsifying, buffering, wetting, solubilizing, stabilizing, flavoring and sweetening agents. Liquid vehicle may include PBS buffer, saline, sucrose or a suitable polyhydric alcohol or alcohols and which optionally contain ethanol, an elixir or linctus.

The inert substances with solubilizing, diluent, emulsifying and/or stabilizing action are well known and conventional and are those generally used for the preparation of pharmaceutical preparations for topical use. Some examples of such inert substances are cetylic alcohol, polyethylene glycol (having for example a molecular weight of 1000)-monocetyl ether, vaseline oil, dimethicone, and propylene glycol.

In some embodiments, the biosensor further comprises a solid support, wherein the R1 of the β-lactam component is covalently bound to the solid support.

In some embodiments, the biosensor further comprises a solid support, wherein the spectroscopic probe is covalently bound to the solid support.

In some embodiments, the solid support is selected from the group consisting of a particle, fiber, an electrospun nanofiber, a microgel, a wound dressing, a catheter, a membrane, a resin, a sponge, a sheet, a suture, an implant scaffold, a stent, a swab, a hydrogel, a film, a patch, a woven fabric, and a nonwoven fabric.

In some embodiments, the solid support is a paper impregnated with a biosensor solution in a solvent such as volatile solvent including dichloromethane, ethylacetate, hexane, methanol, ethanol, or non-volatile solvent including polyethylene glycol, or glycerol.

In some embodiments, the solid support is a hydrogel or microgel impregnated with a biosensor solution in a solvent such as water, aqueous solution of methanol, aqueous solution of ethanol, polyethylene glycol, or glycerol.

In some embodiments, the solid support is a wound dressing, a woven fabric or a nonwoven fabric impregnated with a biosensor solution in a solvent such as volatile solvent including dichloromethane, ethyl acetate, hexane, methanol, ethanol, or non-volatile solvent including polyethylene glycol, or glycerol.

In some embodiments, the concentration of the biosensor provided in the pharmaceutical compositions of the invention is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v.

In some embodiments, the concentration of the biosensor provided in the pharmaceutical compositions of the invention is independently greater than 90%, 80%, 70%, 60%, 50%, 40%, 30%, 20%, 19.75%, 19.50%, 19.25%, 19%, 18.75%, 18.50%, 18.25% 18%, 17.75%, 17.50%, 17.25%, 17%, 16.75%, 16.50%, 16.25%, 16%, 15.75%, 15.50%, 15.25%, 15%, 14.75%, 14.50%, 14.25%, 14%, 13.75%, 13.50%, 13.25% 13%, 12.75%, 12.50%, 12.25%, 12%, 11.75%, 11.50%, 11.25% 11%, 10.75%, 10.50%, 10.25%, 10%, 9.75%, 9.50%, 9.25%, 9%, 8.75%, 8.50%, 8.25%, 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1.0%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of the biosensor provided in the pharmaceutical compositions of the invention is independently in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, or about 1% to about 10% w/w, w/v or v/v.

In some embodiments, the concentration of the biosensor provided in the pharmaceutical compositions of the invention is independently in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9 w/w, w/v or v/v.

### Dermatological Composition

The present invention is related to formulations containing a colorimetric biosensor useful for the rapid detection of drug-resistant microbes like MRSA using visible color-change technology as well as the treatment of microbial infection conditions using a light-stimulated chemical reaction.

The herein described formulation/composition (e.g., a hand sanitizer, or a hand wash composition) containing the colorless diagnostic compound (biosensor) in a dermatologically compatible carrier may be applied to the hands of a person (could be a health care worker, patient or visitor) or the localized infection site in a patient or a specialized equipment that may be suspected of carrying drug-resistant microbes. If drug-resistant microbes are present, diagnostic compound changes from colorless to a bright color (for e.g. blue or violet) which can be easily identified. The diagnostic compound can be formulated in a variety of carriers from gels and creams to powders and liquid solutions. For example: if MRSA is present on the hands of a HCW or at the infection site in a patient, the gel will turn a vibrant color. If MRSA is not present, the gel will not change color. These diagnostic compounds can be formulated in a gel or cream or any other formulation that can be easily spread on the hands of a patient, health care worker or a visitor or any other individual. Specifically, the molecules can be added in currently used hand sanitizers, soaps or creams for rapidly indicating the presence of drug-resistant bugs and sanitizing or disinfecting them.

In some embodiments, the disclosure provides a dermatological composition for administering a biosensor to an infection site in a subject. In some embodiments, the dermatological composition comprises the biosensor disclosed herein and a dermatologically acceptable excipient typically in the form of gel, powder, creams, lotion, ointment, emulsion, or liquid formulation. In some embodiments, the dermatological formulation may additionally comprise a dermatologically acceptable excipient including solvents, dispersion media, diluents, or other liquid vehicles, dispersion or suspension aids, surface active agents, isotonic agents, thickening or emulsifying agents, preservatives, solid binders, lubricants, antimicrobial preservatives, antioxidants and other excipients such as dispersing, suspending, thickening, emulsifying, buffering, wetting, solubilizing, stabilizing, flavoring and sweetening agents. Liquid vehicle may include PBS buffer, saline, sucrose or a suitable polyhydric alcohol or alcohols and which optionally contain ethanol, an elixir or linctus.

The inert substances with solubilizing, diluent, emulsifying and/or stabilizing action are well known and conventional and are those generally used for the preparation of dermatological preparations for topical use. Some examples of such inert substances are cetylic alcohol, polyethylene glycol (having for example a molecular weight of 1000)-monocetyl ether, vaseline oil, dimethicone, and propylene glycol.

In some embodiments, the biosensor further comprises a solid support, wherein the β-lactam component is covalently bound to the solid support.

In some embodiments, the biosensor further comprises a solid support, wherein the spectroscopic probe is covalently bound to the solid support.

In some embodiments, the solid support is selected from the group consisting of a particle, fiber, a microgel, a wound dressing, a catheter, a membrane, a resin, a sponge, a sheet, a suture, an implant scaffold, a stent, a swab, a hydrogel, a film, a patch, a tape, a woven fabric, and a nonwoven fabric.

In some embodiments, the solid support is a paper impregnated with a biosensor solution in a solvent such as volatile solvent including dichloromethane, ethylacetate, hexane, methanol, ethanol, or non-volatile solvent including polyethylene glycol, or glycerol.

In some embodiments, the solid support is a hydrogel or microgel impregnated with a biosensor solution in a solvent such as water, aqueous solution of methanol, aqueous solution of ethanol, polyethylene glycol, or glycerol.

In some embodiments, the solid support is a wound dressing, a woven fabric or a nonwoven fabric impregnated with a biosensor solution in a solvent such as volatile solvent including dichloromethane, ethylacetate, hexane, methanol, ethanol, or non-volatile solvent including polyethylene glycol, or glycerol.

In some embodiments, the concentration of the biosensor provided in the dermatological compositions of the invention is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v.

In some embodiments, the concentration of the biosensor provided in the dermatological compositions of the invention is independently greater than 90%, 80%, 70%, 60%, 50%, 40%, 30%, 20%, 19.75%, 19.50%, 19.25%, 19%, 18.75%, 18.50%, 18.25% 18%, 17.75%, 17.50%, 17.25%, 17%, 16.75%, 16.50%, 16.25%, 16%, 15.75%, 15.50%, 15.25%, 15%, 14.75%, 14.50%, 14.25%, 14%, 13.75%, 13.50%, 13.25%, 13%, 12.75%, 12.50%, 12.25%, 12%, 11.75%, 11.50%, 11.25%, 11%, 10.75%, 10.50%, 10.25%, 10%, 9.75%, 9.50%, 9.25%, 9%, 8.75%, 8.50%, 8.25%, 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1.0%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of the biosensor provided in the dermatological compositions of the invention is independently in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, or about 1% to about 10% w/w, w/v or v/v.

In some embodiments, the concentration of the biosensor provided in the dermatological compositions of the invention is independently in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9 w/w, w/v or v/v.

### Dendrimer Biosensor

In some embodiments, the solid support is a microgel comprising a dendritic polymer. Dendrimers are also classified by generation, which refers to the number of repeated branching cycles that are performed during its synthesis. For example, if a dendrimer is made by convergent synthesis, and the branching reactions are performed onto the core molecule three times, the resulting dendrimer is considered a third generation dendrimer (G3). Each successive generation results in a dendrimer roughly twice the molecular weight of the previous generation. Higher generation dendrimers also have more exposed functional groups on the surface, which can later be used to customize the dendrimer for a given application.

In some embodiments, the dendritic polymer is selected from hyperbranched PEG dendrimers, PEG core dendrimers, hyperbranched polyglycerol dendrimers, hyperbranched polylysine dendrimers, hyperbranched polyesters, alkyne-terminated dendrimers, amine terminated PEG-core dendrimers, azide terminated dendrimers, 2,2-bis(methylol)propionic acid (bis-MPA) dendrimers, carboxylic acid terminated dendrimers, Poly(amidoamine) (PAMAM) dendrimers, polyethylenimine dendrimers (PEI), and combinations thereof.

In some embodiments, the dendritic polymer has reactive surface group available for biosensor conjugation selected from the group consisting of 8 surface groups, 16 surface groups, 32 surface groups, 64 surface groups, and 128 surface groups. In some embodiments, the reactive surface groups carried by the dendritic polymer is selected from the group consisting of (—CH═CH₂), ethynyl group (—C≡C—), azide group (—N₃), vinyl dimethyl sulfone group, hydroxyl group (—OH), thiol group (—SH), amine group (—NH₂), aldehyde group (—CHO), carboxylic acid group (—COOH), and combinations thereof.

In some embodiments, the dendrimer is a polyester bis-MPA dendrimer (tert-butylic acid protected amine core, 8 alkyne end groups, G3, branching units bis-MPA). These alkyne-functionalized dendrimers can be readily functionalized using either copper (I)-catalyzed alkyne-azide cycloaddition (CuAAC), strain-promoted alkyne-azide cycloaddition (SPAAC), or thiol-yne click reactions. Additionally, the amine-functionalized core can be readily used in EDC or DCC coupling reactions (after Boc deprotection) with carbonyl-containing compounds to yield highly functionalized materials for a variety of biomedical applications. In some embodiments, the biosensor as disclosed herein is modified with an azide group and is conjugated with the G3 polyester bis-MPA dendrimer via click chemistry.

In some embodiments, the dendritic polymer is selected from the group consisting of bis-MPA hyperbranched PEG10k-OH dendrimer (10K PEG core, pseudo generation 2, bis-MPA branching units, 8 surface hydroxyl groups, Mw 10696 Da), bis-MPA hyperbranched PEG10k-OH dendrimer (10K PEG core, pseudo generation 3, bis-MPA branching units, 16 surface hydroxyl groups, Mw 11643 Da), bis-MPA hyperbranched PEG20k-OH dendrimer (20K PEG core, pseudo generation 2, bis-MPA branching units, 8 surface hydroxyl groups, Mw 20759 Da), bis-MPA hyperbranched PEG20k-OH dendrimer (20K PEG core, pseudo generation 3, bis-MPA branching units, 16 surface hydroxyl groups, Mw 21688 Da), bis-MPA hyperbranched PEG6k-OH dendrimer (6K PEG core, pseudo generation 4, bis-MPA branching units, 32 surface hydroxyl groups, Mw 9480 Da), and combinations thereof. In some embodiments, the bis-MPA hyperbranched dendrimer forms microparticle or microgel.

In some embodiments, the dendritic polymer is selected from the group consisting of G2 polylysine, G3 polylysine, G4 polylysine, G5 polylysine, and G6 polylysine.

In some embodiments, a spacer can link the biosensor to the dendrimer. In some embodiments, the spacer links biosensor and dendrimer via an amide bond formed by NHS/EDC chemistry. In some embodiments, the spacer links biosensor and the dendrimer via a disulfide (S—S) bond. In some embodiments, the spacer links biosensor and the dendrimer via triazoles formed by copper(I)-catalyzed alkyne-azide cycloaddition (CuAAC), or strain-promoted alkyne-azide cycloaddition (SPAAC), or thiol-yne click reactions

In some embodiments, the biosensor comprises β-lactam inhibitor fragment -(amino-(spacer)x)y-dendrimer or dendrimer-(spacer)z-β-lactam antibiotic, wherein the spacer has from 2 to 50 atoms, x, y and z are integers from 5 to 15.

In some embodiments, the spacer joined dendrimer and the β-lactam antibiotic fragment via a degradable bond is selected from the group consisting of an ester bond, an amide bond, an imine bond, an acetal bond, a ketal bond, and combinations thereof.

In some embodiments, the spacer is selected from the group consisting of polyethylene glycol having 2-50 repeating units, ε-maleimidocaproic acid, para-aminobenzyloxy carbamater, and combinations thereof. In some embodiments, the spacer comprises polyamino acid having 2-30 amino acid residues. In some embodiments, the spacer comprises a linear polylysine, or polyglutamine.

### Diagnostic Particle

In some embodiments, the biosensor as disclosed herein is encapsulated within or covalently conjugated to a particle to form a diagnostic particle for microbial detection. In some embodiments, the biosensor is conjugated to the surface of the diagnostic particle. In some embodiments, the biosensor is conjugated to the interior of the diagnostic particle.

In some embodiments, the diagnostic particle comprises a bland particle core coated with the biosensor as disclosed herein. For example, in some embodiments, the diagnostic particle comprises an iron oxide nanoparticle coated with the biosensor disclosed herein. In some embodiments, the diagnostic particle comprises a gold nanoparticle coated with the biosensor disclosed herein.

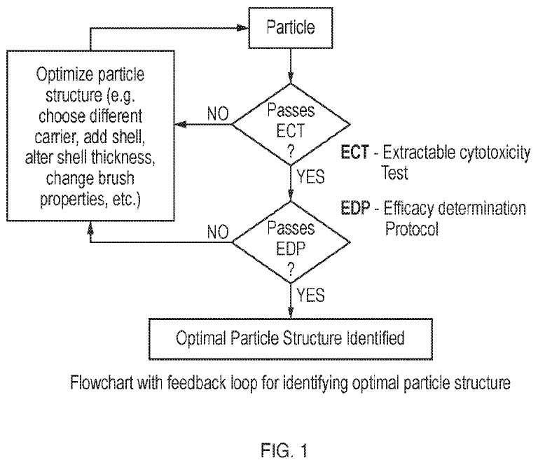

In an embodiment, the diagnostic particle comprises: (1) a carrier and (2) the biosensor as disclosed herein wherein the first material is covalently conjugated to the spectroscopic probe, wherein the first material masks the optical activity of the spectroscopic probe, wherein the antimicrobial inactivating factor cause degradation of the first material to release the spectroscopic probe and result in a detectable optical response, and further wherein the particle structure is constructed such that it passes the Extractable Cytotoxicity Test. In some embodiments, the particle is designed in accordance with a feedback loop illustrated in the flowchart of FIG. 1.

In some embodiments, the diagnostic particle further passes the Efficacy Determination Protocol.

In some embodiments, the antimicrobial inactivating factors secreted by the microbes are selected from the group consisting of β-lactamase, microbial hydrolase, microbial esterase, and combinations thereof. In some embodiments, the antimicrobial inactivating factor is selected from the group consisting of β-lactamase, erythromycin (macrolide) esterase, chloramphenicol (phenicol) hydrolase, and combinations thereof.

In some embodiments, the antimicrobial inactivating factor is a β-lactamase secreted by the antibiotic resistant microbes. In some embodiments, the β-lactamase is selected from the family of Class A serine carbapenmase, class B-D β-lactamase, ESBL, metallo-β-lactamases, AmpC cephalosporinases, and combinations thereof. In some embodiments, the antimicrobial inactivating factor is an erythromycin (macrolide) esterase secreted by the antibiotic resistant microbes. In some embodiments, the antimicrobial inactivating factor is a chloramphenicol (phenicol) hydrolase secreted by the antibiotic resistant microbes.

In some embodiments, the biosensor in the diagnostic particle is a compound selected from those disclosed in Table 1.

In some embodiments, at least a portion of the exterior surface of the diagnostic particle has a modification that is polar, non-polar, charged, ionic, basic, acidic, reactive, hydrophobic, or hydrophilic.

In some embodiments, a spacer can link the biosensor to the surface of the particle to form a diagnostic particle. In some embodiments, the spacer links biosensor and the particle via an amide bond formed by NHS/EDC chemistry to form the diagnostic particle. In some embodiments, the spacer links biosensor and the particle via a disulfide (S—S) bond to form the diagnostic particle. In some embodiments, the spacer links the biosensor and the particle via triazoles formed by copper(I)-catalyzed alkyne-azide cycloaddition (CuAAC), or strain-promoted alkyne-azide cycloaddition (SPAAC), or thiol-yne click reactions to form the diagnostic particle.

In some embodiments, the diagnostic particle comprises β-lactam inhibitor fragment -(amino-(spacer)x)y- particle or particle-(spacer)z-β-lactam antibiotic, wherein the spacer has from 2 to 50 atoms, x, y and z are integers from 5 to 15.

In some embodiments, the spacer that joins the particle and the β-lactam antibiotic fragment via a degradable bond to form the diagnostic particle is selected from the group consisting of an ester bond, an amide bond, an imine bond, an acetal bond, a ketal bond, and combinations thereof.

In some embodiments, the spacer is selected from the group consisting of polyethylene glycol having 2-50 repeating units, ε-maleimidocaproic acid, para-aminobenzyloxy carbamater, and combinations thereof. In some embodiments, the spacer comprises polyamino acid having 2-30 amino acid residues. In some embodiments, the spacer comprises a linear polylysine, or polyglutamine.

In some embodiments, the diagnostic particle is porous and the pores of the diagnostic particle are plugged with a protein or peptide degradable by enzymes secreted by the microbes. When the protein/peptide plugged diagnostic particle meets the liquid medium of the testing sample, the enzyme secreted by the microbes causes degradation of the protein/peptide plug such that the diagnostic particle becomes permeable to the liquid medium of the testing sample. In some embodiments, the porous particle comprises mesoporous silica, or zeolite nanoparticles.

In some embodiments, the diagnostic particle may further comprise a shell to form a core-shell diagnostic particle. In some embodiments, the shell is formed from an agent selected from protein, polysaccharide, lipid, mesoporous silica, and combinations thereof.

In some embodiments, the diagnostic particle comprises a porous diagnostic particle impregnated with the biosensor as disclosed herein and a shell degradable by the enzymes secreted by the microbes. In some embodiments, the enzymes secreted by the microbes include microbial proteases, metallo-protease, collagenase, esterases, hyaluronidase, or hydrolysase. In some embodiments, the shell is composed of a biodegradable polymer selected from the group consisting of collagen, gelatin, ovalbumin, serum albumin, hyaluronate, hyaluronic acid, lipopolysaccharide (LPS), and combinations thereof.

In some embodiments, the diagnostic particle comprises a porous diagnostic particle impregnated with the biosensor as disclosed herein and a shell degradable by glutathione. In some embodiments, the shell degradable by glutathione comprises a crosslinked polysperine with disulfide bond as crosslinkers. In some embodiments, the crosslinked polysperine is a reaction product of spermine and a crosslinking reagent in a molar ratio of about 5:1 to about 1:5. The resulting polyspermine polymer can, in certain embodiments, have a molecular weight of about 1 kDa to about 1,000 kDa. A 1:1 molar ratio of spermine and the crosslinking reagent can form the highest molecular weight polymer of polyspermine.

In some embodiments, the shell is porous and the pores of the shell are plugged with a protein or peptide degradable by enzymes secreted by the microbes. When the protein/peptide plugged shell is contacted with the liquid medium of the testing sample, the enzyme secreted by the microbes cause degradation of the protein/peptide plug such that the diagnostic particle permeable to the liquid medium of the testing sample. In some embodiments, the enzymes secreted by the microbes include microbial proteases, metallo-protease, collagenase, esterases, hyaluronidase, or hydrolysase. In some embodiments, the shell is composed of a biodegradable polymer selected from the group consisting of collagen, gelatin, ovalbumin, serum albumin, hyaluronate, hyaluronic acid, lipopolysaccharide (LPS), and combinations thereof.

In some embodiments, the shell is porous and the pores of the shell are plugged with a substance degradable by glutathione. In some embodiments, the plug degradable by glutathione comprises a crosslinked polysperine with disulfide bond as crosslinkers. In some embodiments, the crosslinked polysperine is a reaction product of spermine and a crosslinking reagent in a molar ratio of about 5:1 to about 1:5. The resulting polyspermine polymer can, in certain embodiments, have a molecular weight of about 1 kDa to about 1,000 kDa. A 1:1 molar ratio of spermine and the crosslinking reagent can form the highest molecular weight polymer of polyspermine.

In some embodiments, the crosslinking reagent is selected from the group consisting of dithiobis(succinimidyl propionate (Lomant's reagent, or “DSP”), cystamine bisacrylamide, bisacryloyloxyethyl) disulfide, dimethyl 3,3′-dithiobispropionimidate, bis-((β)-(4-azidosalicylamido)ethyl)disulfide, 4-succinimidyloxycarbonyl-α-methyl-α-(2-pyridyldithio)toluene, and combinations thereof.

In some embodiments, the shell may comprise inorganic polymers such as silicates, organosilicate, organo-modified silicone polymer, or may be crosslinked organic polymers such as polyureas or polyurethanes. The process to apply the crosslinked shell must be designed so as to maximize the stability of the diagnostic particle components to the chemistry required in shell construction, at least until the growing shell protects the components encapsulated in the diagnostic particle.

Therefore, in some embodiments, the present disclosure provides diagnostic particles having a core-shell structure to reduce diagnostic particle porosity and to protect the first material from the degradation by the body chemicals. Therefore, the stability of the first material inside the diagnostic particle is improved due to the reduced incursion of the body chemicals. In some embodiments, the shell comprises a crosslinked organo-silicate polymer derived from trialkoxysilane, or trihalorosilane. For example, to protect the biosensor encapsulated in a poly(methyl methacrylate-co-butyl methacrylate) 96:4 (96:4 MMA-BMA) diagnostic particle when introduced into the liquid medium of testing sample, a sol-gel organo-modified silicate polymer shell derived from alkyltrimethoxysilane is formed on the surface of the polymeric diagnostic particle to block the free exchange of nucleophiles between the diagnostic particles and the surrounding environment other than testing sample site.

In some embodiments, the trialkoxysilane used for making the shell is selected from the group consisting of C2-C7 alkyl-trialkoxysilane, C2-C7 alkenyl-trialkoxysilane, C2-C7 alkynyl-trialkoxysilane, aryl-trialkoxysilane, and combinations thereof. In some embodiments, the trihalosilane used for making the shell is selected from the group consisting of trichlorosilane, tribromosilane, triiodosilane, and combinations thereof. In some embodiments, the crosslinked organo-silicate polymer is derived from vinyl-trimethoxysilane.

In some embodiments, the diagnostic particles are formulated as topical formulations for detecting drug resistant microbes in cutaneous infection (e.g., a wound dressing impregnated with a liquid dispersion of diagnostic particles). In some embodiments, the disclosure provides topical diagnostic formulations suitable for the detection of the drug resistant microbes at a cutaneous infection site. In some embodiments, the topical diagnostic formulation may take the form selected from the group consisting of a cream, a lotion, an ointment, a hydrogel, a colloid, a gel, a foam, an oil, a milk, a suspension, a wipe, a sponge, a solution, an emulsion, a paste, a patch, a tape, a pladget, a swab, a dressing, a spray, a pad, and combinations thereof.

### (a) Carrier

In some embodiments, the diagnostic particle comprises a carrier selected from the group consisting of a lipid, polymer-lipid conjugate, carbohydrate-lipid conjugate, peptide-lipid conjugate, protein-lipid conjugate, an inorganic polymer, a polyester, a polyurea, a polyanhydride, a polysaccharide, a polyphosphoester, a poly(ortho ester), a poly(amino acid), a protein, dendritic polylysine, and combinations thereof.

In an embodiment, the carrier may include a lipid selected from the group consisting of lipid, polymer-lipid conjugate, carbohydrate-lipid conjugate, peptide-lipid conjugate, protein-lipid conjugate, and combinations thereof. In some embodiments, the lipid has a melting temperature (Tm) ranging from about 35° C. to about 120° C. In some embodiments, the lipid has a melting temperature Tm ranging from about 55° C. to about 60° C.

In some embodiments, the lipid is selected from the group consisting of 1,2-dipalmitoyl-sn-glycerol-3-phospho-(1′-rac-glycerol) (DPPG), 1,2-distearoyl-sn-glycerol-3-phosphoglycerol, sodium salt (DSPG), 1,2-dimyristoyl-sn-glycero-3-phospho-L-serine sodium salt (DMPS, 14:0 PS), 1,2-dipalmitoyl-sn-glycero-3-phosphoserine, sodium salt (DPPS, 16:0 PS), 1,2-distearoyl-sn-glycero-3-phospho-L-serine (sodium salt) (DSPS, 18:0 PS), 1,2-dimyristoyl-sn-glycero-3-phosphate, sodium salt (DMPA, 14:0 PA), 1,2-dipalmitoyl-sn-glycero-3-phosphate, sodium salt (DPPA, 16:0 PA), 1,2-distearoyl-sn-glycero-3-phosphate, sodium salt (DSPA, 18:0), 1′,3′-bis[1,2-dipalmitoyl-sn-glycero-3-phospho]-glycerol sodium salt (16:0 cardiolipin), 1,2-dimyristoyl-sn-glycero-3-phosphoethanolamine (DMPE, 12:0 PE), 1,2-dipalmitoyl-sn-glycero-3-phosphoethanolamine (DPPE, 16:0), 1,2-distearoyl-sn-glycero-3-phosphoethanolamine (DSPE, 18:0), 1,2-diarachidyl-sn-glycero-3-phosphoethanolamine (20:0 PE), 1-stearoyl-2-linoleoyl-sn-glycero-3-phosphoethanolamine, 1,2-dipalmitoyl-sn-glycero-3-phosphocholine (DPPC, 16:0 PC), 1,2-diheptadecanoyl-sn-glycero-3-phosphocholine (17:0 PC), 1,2-distearoyl-sn-glycero-3-phosphocholine (DSPC, 18:0 PC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-myristoyl-2-stearoyl-sn-glycero-3-phosphocholine (14:0-18:0 PC), 1-stearoyl-2-palmitoyl-sn-glycero-3-phosphocholine (16:0-18:0 PC), and combinations thereof.

In one embodiment, the phospholipid is selected from the group consisting of dipalmitoylphosphatidylcholine (DPPC), 1-palmitoyl-2-hydroxyl-sn-glycero-3-phosphocholine (MPPC), 1-myristoyl-2-stearoyl-sn-glycero-3-phosphocholine (MSPC), 1,2-dimyristoyl-sn-glycero-3-phosphocholine (DMPC), 1,2-dimyristoyl-sn-glycero-3-phosphorylglycerol (DMPG), 1,2-distearoyl-sn-glycero-3-phosphoethanolamine (DSPE), 1,2-dioleoyl-sn-glycero-3-phosphocholine (DOPC), 1,2-dioleoyl-sn-glycero-3-phosphoethanolamine (DOPE), 1,2-dipalmitoyl-sn-glycero-3-phospho-(1′-rac-glycerol) (DPPG), 1,2-distearoyl-sn-glycero-3-phosphocholine (DSPC), distearoylphosphoethanolamine conjugated with polyethylene glycol (DSPE-PEG), phosphatidylserine (PS), phosphatidylethanolamine (PE), phosphatidylglycerol (PG), phosphatidylcholine (PC), and combinations thereof. In an embodiment, the particle comprise the lipid selected from the group consisting of DPPC, MPPC, PEG, DMPC, DMPG, DSPE, DOPC, DOPE, DPPG, DSPC, DSPE-PEG, MSPC, cholesterol, PS, PC, PE, PG, and combinations thereof.

In some embodiments, the lipid comprises a thermoresponsive lipid/polymer hybrid. In some embodiments, the thermoresponsive lipid/polymer hybrid is selected from the group consisting of composite containing triblock copolymer of [poly(2-isopropyl-2-oxazoline)-b-poly(dimethylsiloxane)-b-poly(2-isopropyl-2-oxazoline] and 1,2-dimyristoyl-sn-glycero-3-phosphocholine (DMPC), composite containing block copolymers poly(cholesteryl acrylate)-b-poly(N-isopropylacrylamide) (PNIPAAm) and lipid, and combinations thereof.

In an embodiment, the carrier may include a lipid selected from the group consisting of lipid, polymer-lipid conjugate, carbohydrate-lipid conjugate, peptide-lipid conjugate, protein-lipid conjugate, and combinations thereof. In some embodiments, the lipid may include one or more of the following: phospholipids such as phosphatidylcholines, phosphatidylserines, phosphatidylinositides, phosphatidylethanolamines, phosphatidylglycerols, phosphatidic acids; sphingolipids such as sphingomyelins, ceramides, phytoceramides, cerebrosides; sterols such as cholesterol, desmosterol, lanthosterol, stigmasterol, zymosterol, or diosgenin.

In some embodiments, the carrier comprises a polymer-lipid conjugate, wherein the polymers conjugated to polar head groups of the lipid may include polyethylene glycol, polyoxazolines, polyglutamines, polyasparagines, polyaspartamides, polyacrylamides, polyacrylates, polyvinylpyrrolidone, or polyvinylmethyether. In some embodiments, the carrier comprises a carbohydrate-lipid conjugate, wherein the carbohydrates conjugated to the lipid may include monosaccharides (glucose, fructose), disaccharides, oligosaccharides or polysaccharides such as glycosaminoglycan (hyaluronic acid, keratan sulfates, heparin sulfate or chondroitin sulfate), carrageenan, microbial exopolysaccharides, alginate, chitosan, pectin, chitin, cellulose, or starch.

In some embodiments, the carrier comprises an inorganic agent. In some embodiments, the inorganic agent is selected from the group consisting of iron oxide nanoparticle, gold nanoparticle, mesoporous silica, apatite, hydroxyapatite, hydroxycarbonate apatite, calcium carbonate, calcium phosphate including monocalcium phosphate, dicalcium phosphate, tricalcium phosphate, and tetracalcium phosphate, and combinations thereof.

In an embodiment, the carrier comprises a polymer. In some embodiments, the polymer is a biocompatible polymer and/or a biodegradable polymer. In some embodiments, the carrier comprises a biodegradable polymer. In some embodiments, the carrier comprises a biocompatible polymer.

In some embodiments, the carrier may include, but are not limited to, a polyester, a polyurea, a polyanhydride, a polysaccharide, a polyphosphoester, a poly(ortho ester), a poly(amino acid), a protein, and combinations thereof.

In one embodiment, the carrier comprises a polyester. Polyesters are a class of polymers characterized by ester linkages in the backbone, such as poly (lactic acid) (PLA), poly(glycolic acid) (PGA), PLGA, etc. PLGA is one of the commonly used polymers in developing particulate drug delivery systems. PLGA degrades via hydrolysis of its ester linkages in the presence of water.

In some embodiments, the carrier is selected from the group consisting of poly(lactic acid) (PLA), poly(glycolic acid) (PGA), PLGA, poly(lactic acid)-polyethylene glycol-poly(lactic acid) (PLA-PEG-PLA), poly (L-co-D, L lactic acid) (PLDLA); poly-L-lactic acid-co-glycolic acid, poly-D,L-lactic acid-co-glycol acid; poly-valerolacton, poly-hydroxyl butyrate and poly-hydroxyl valerate, polycaprolactone (PCL), γ-polyglutamic acid graft with poly (L-phenylalanine) (γ-PGA-g-L-PAE), poly(cyanoacrylate) (PCA), polydioxanone, poly(butylene succinate), poly(trimethylene carbonate), poly(p-dioxanone), poly(butylene terephthalate), poly(P-hydroxyalkanoate)s, poly(hydroxybutyrate), and poly(hydroxybutyrate-co-hydroxyvalerate), poly (ε-lysine), poly-L-lysine (PLL), poly(valeric acid), and poly-L-glutamic acid, poly(ester amide), poly(ester ether) diblock copolymer of poly(sebacic acid) and polyethylene glycol (PSA-PEG), trimethylene carbonate, poly(β-hydroxybutyrate), poly(g-ethyl glutamate), poly(desaminotyrosinetyrosylhexyl iminocarbonate), poly(bisphenol A iminocarbonate), polyphosphazene, collagen, albumin, gluten, chitosan, hyaluronate, hyaluronic acid, cellulose, alginate, starch, gelatin, pectin, and combinations thereof.

In some embodiments, the carrier is selected from the group consisting of poly (lactic acid) (PLA); poly(glycolic acid) (PGA); poly(lactide-co-glycolide) (PLGA); block copolymer of polyethylene glycol-b-poly lactic acid-co-glycolic acid (PEG-PLGA); polycaprolactone (PCL); poly-L-lysine (PLL); random graft co-polymer with a poly(L-lysine) backbone and poly(ethylene glycol) (PLL-g-PEG); dendritic polymer including polyethyleneimine (PEI) and derivatives thereof, dendritic polyglycerol and derivatives thereof, dendritic polylysine; and combinations thereof.

In some embodiments, the carrier comprises polyester selected from the group consisting of PLA, PGA, PLGA, and combinations thereof.

In some embodiments, copolymers of PEG or derivatives thereof with any of the polymers described above may be used as carrier to make the polymeric particles. In some embodiments, the carrier comprises a polymer blend containing PLGA 75:25 and PLGA-PEG 75:25 with lactide:glycolide monomer ratio of 75:25.

In some embodiments, the carrier comprises PEG grafted dendritic polymer PEI, polyglycerol, and polylysine. In some embodiments, the carrier comprises PEG grafted dendritic polymer PEI, polyglycerol, and polylysine, wherein the PEG is terminated with a reactive functional group selected from the group consisting of vinyl group (—CH═CH₂), ethynyl group (—C≡C—), vinyl dimethyl sulfone group, hydroxyl group (—OH), thiol group (—SH), amine group (—NH₂), aldehyde group (—CHO), carboxylic acid group (—COOH), and combinations thereof. In some embodiments, the carrier comprises PEG grafted dendritic polymer PEI, polyglycerol, and polylysine, wherein the PEG is terminated with an amine group (—NH₂), wherein the amine group becomes cationically charged under acidic conditions (e.g., pH=4-6). In some embodiments, the carrier comprises PEG grafted dendritic polymer PEI, polyglycerol, and polylysine, wherein the PEG is terminated with a thiol group (—SH).

In some embodiments, the PEG or derivatives may locate in the interior positions of the triblock copolymer (e.g. PLA-PEG-PLA). Alternatively, the PEG or derivatives may locate near or at the terminal positions of the block copolymer. In some embodiments, the nanoparticles are formed under conditions that allow regions of PEG to phase separate or otherwise to reside on the surface of the particles.

In some embodiments, the carrier comprises PLGA. PLGA denotes a copolymer (or co-condensate) of lactic acid and glycolic acid. The PLGA copolymers for use in the present invention are preferably biodegradable, i.e. they degrade in an organism over time by enzymatic or hydrolytic action or by similar mechanisms, thereby producing pharmaceutically acceptable degradation products, and biocompatible, i.e. that do not cause toxic or irritating effects or immunological rejection when brought into contact with a body fluid. The lactic acid units may be L-lactic acid, D-lactic acid or a mixture of both.

In some embodiments, the polymethacrylate copolymer is MMA/BMA copolymer and the weight ratio of MMA to BMA is 96:4 (e.g. NeoCryl® 805 by DSM, acid value less than 1). In one embodiment, the carrier is poly(methyl methacrylate) (PMMA). In some embodiments, the carrier is a polyacrylate blend comprising 96% poly(methyl methacrylate) and 4% poly(butyl methacrylate). In some embodiments, the carrier is a methyl methacrylate/butyl methacrylate copolymer comprising 96% methyl methacrylate repeating units and 4% butyl methacrylate repeating units. In some embodiments, the carrier is a copolymer of methyl methacrylate/butyl methacrylate (NeoCryl® B-805, Tg 99° C., average molecular weight 85,000 Da).

In some embodiments, the carrier comprises cross-linkable reactive groups selected from vinyl group (—CH═CH2), ethynyl group (—C≡C—), vinyl dimethyl sulfone group, hydroxyl group (—OH), thiol group (—SH), amine group (—NH2), aldehyde group (—CHO), carboxylic acid group (—COOH), and combinations thereof. In some embodiments, the carrier comprises cross-linkable polysaccharides.

In some embodiments, the carrier is present in the diagnostic particle at a weight percentage by the total weight of the diagnostic particle selected from the group consisting of about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, or about 20.0 wt. %, about 25.0 wt. %, about 30.0 wt. %, about 35.0 wt. %, about 40.0 wt. %, about 45.0 wt. %, about 50.0 wt. %, about 55.0 wt. %, about 60.0 wt. %, about 65.0 wt. %, about 70.0 wt. %, about 75.0 wt. %, about 80.0 wt. %, about 85.0 wt. %, about 90.0 wt. %, about 95.0 wt. %, and about 99.0 wt. %. In some embodiments, the carrier is present in the diagnostic particle at a weight percentage by the total weight of the diagnostic particle ranging from about 1.0 wt. % to about 99.0 wt. %. In some embodiments, the carrier is present in the diagnostic particle at a weight percentage by the total weight of the diagnostic particle ranging from about 10.0 wt. % to about 90.0 wt. %. In some embodiments, the carrier is present in the diagnostic particle at a weight percentage by the total weight of the diagnostic particle ranging from about 50.0 wt. % to about 90.0 wt. %. In some embodiments, the carrier is present in the diagnostic particle at a weight percentage by the total weight of the diagnostic particle ranging from about 25.0 wt. % to about 50.0 wt. %. In some embodiments, the carrier is present in the diagnostic particle at a weight percentage by the total weight of the diagnostic particle ranging from about 75.0 wt. % to about 90.0 wt. %.

In some embodiments, the diagnostic particle comprises NeoCryl® B-805 (copolymer of 96.0 wt. % methyl methacrylate/4.0 wt. % butyl methacrylate) in an amount ranging from about 60.0 wt. % to about 80 wt. % by the total weight of the diagnostic particle. In some embodiments, the diagnostic particle comprises NeoCryl® B-805 in an amount selected from the group consisting of 62.0 wt. %, 70.0 wt. %, 75.0 wt. %, and 78.3 wt. % by the total weight of the diagnostic particle. In some embodiments, the diagnostic particle comprises NeoCryl® B-805 in an amount selected from the group consisting of about 55.0 wt. %, about 56.0 wt. %, about 57.0 wt. %, about 58.0 wt. %, about 59.0 wt. %, about 60.0 wt. %, about 61.0 wt. %, about 62.0 wt. %, about 63.0 wt. %, about 64.0 wt. %, about 65.0 wt. %, about 66.0 wt. %, about 67.0 wt. %, about 68.0 wt. %, about 69.0 wt. %, about 70.0 wt. %, about 71.0 wt. %, about 72.0 wt. %, about 73.0 wt. %, about 74.0 wt. %, about 75.0 wt. %, about 76.0 wt. %, about 77.0 wt. %, about 78.0 wt. %, about 79.0 wt. %, and about 80 wt. % by the total weight of the diagnostic particle.

In some embodiments, the biosensor in the diagnostic particle is present in an amount ranging from about 5.0 wt. % to about 15.0 wt. % by the total weight of the diagnostic particle. In some embodiments, the biosensor of the diagnostic particle is present in an amount selected from the group consisting of about 5.0 wt. %, about 5.56 wt. %, about 10.4 wt. %, about 12.0 wt. %, about 12.1 wt. %, about 13.64 wt. %, about 14.0 wt. %, and about 15.0 wt. % by the total weight of the diagnostic particle. In some embodiments, the diagnostic particle comprises the biosensor in an amount of about 5.0 wt. %, about 5.25 wt. %, about 5.5 wt. %, about 5.75 wt. %, about 6.0 wt. %, 6.25 wt. %, about 6.5 wt. %, about 6.75 wt. %, about 7.0 wt. %, 7.25 wt. %, about 7.5 wt. %, about 7.75 wt. %, about 8.0 wt. %, about 8.25 wt. %, about 8.5 wt. %, about 8.75 wt. %, about 9.0 wt. %, about 9.25 wt. %, about 9.5 wt. %, about 9.75 wt. %, about 10.0 wt. %, about 10.25 wt. %, about 10.5 wt. %, about 10.75 wt. %, about 11.0 wt. %, about 11.25 wt. %, about 11.5 wt. %, about 11.75 wt. %, about 12.0 wt. %, about 12.25 wt. %, about 12.5 wt. %, about 12.75 wt. %, about 13.0 wt. %, about 13.25 wt. %, about 13.5 wt. %, about 13.75 wt. %, about 14.0 wt. %, about 14.25 wt. %, about 14.5 wt. %, about 14.75 wt. %, or about 15.0 wt. %.

In some embodiments, the diagnostic particle has a weight ratio of the carrier to the biosensor ranging from 1:1 to 7:1. In some embodiments, the diagnostic particle has a weight ratio of the carrier to the biosensor selected from the group consisting of 1.0:1, 1.1:1, 1.2:1, 1.3:1, 1.4:1, 1.5:1, 1.6:1, 1.7:1, 1.8:1, 1.9:1, 2.0:1, 2.1:1, 2.2:1, 2.3:1, 2.4:1, 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 41.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, and 7.0:1.

### (b) Optional Additive for Diagnostic Particles

In some embodiments, the diagnostic particle further includes thermal stabilizers. It should be noted that often the second material that interacts with the exogenous source can be stable (low rate of degradation) at room temperature but when the diagnostic particle comprising the second material is inside body, at body temperature of 37.5° C., degradation of the second material can be significantly accelerated. Examples of useful thermal stabilizers include phenolic antioxidants such as butylated hydroxytoluene (BHT), 2-t-butylhydroquinone, and 2-t-butylhydroxyanisole.

In some embodiments, the core of the diagnostic particle may optionally comprise an additive. In some embodiments, the additive is an antioxidant, or a surfactant. In some embodiments, the additive is an antioxidant for stabilizing the dyes. In some embodiments, the antioxidants for stabilizing dyes comprise sterically hindered phenols with para-propionate groups. In some embodiments, the antioxidant for stabilizing dyes comprises pentaerythritol tetrakis(3,5-di-tert-butyl-4-hydroxyhydrocinnamate). In some embodiments, the antioxidant for stabilizing dyes comprises a phosphite such as tris(2,4-di-tert-butylphenyl)phosphite. In some embodiments, the antioxidant for stabilizing dyes comprises organosulfur compounds such as thioethers. In some embodiments, the antioxidant for stabilizing dyes comprises 1,3,5-TR1S(4-tert-butyl-3-hydroxy-2,6-dimethylbenzyl)-1,3,5-triazine-2,4,6-(1H,3H,5H)-trione (Cyanox® 1790); wherein the Cyanox® 1790 is colorless.

In some embodiments, the additive is a surfactant. In some embodiments, the surfactant may include cationic, amphoteric, and non-ionic surfactants. In some embodiments, the surfactants comprise anionic surfactants selected from the group consisting of fatty acid salts, bile salts, phospholipids, carnitines, ether carboxylates, succinylated monoglycerides, mono/diacetylated tartaric acid esters of mono- and diglycerides, citric acid esters of mono- and diglycerides, sodium oleate, sodium lauryl sulfate, sodium lauryl sarcosinate, sodium dioctyl sulfosuccinate (SDS), sodium cholate, sodium taurocholate, lauroyl carnitine, palmitoyl carnitine, myristoyl carnitine, lactylic esters of fatty acids, and combinations thereof. In some embodiments, anionic surfactants include di-(2-ethylhexyl) sodium sulfosuccinate. In some embodiments, the surfactants are non-ionic surfactants selected from the group consisting of propylene glycol fatty acid esters, mixtures of propylene glycol fatty acid esters and glycerol fatty acid esters, triglycerides, sterol and sterol derivatives, sorbitan fatty acid esters and polyethylene glycol sorbitan fatty acid esters, sugar esters, polyethylene glycol alkyl ethers and polyethylene glycol alkyl phenol ethers, polyoxyethylene-polyoxypropylene block copolymers, lower alcohol fatty acid esters, and combinations thereof. In some embodiments, the surfactant may comprise fatty acids. Examples of fatty acids include caprylic acid, undecylic acid, lauric acid, tridecylic acid, myristic acid, palmitic acid, stearic acid, or oleic acid. In some embodiments, the surfactants comprise amphoteric surfactants including (1) substances classified as simple, conjugated and derived proteins such as the albumins, gelatins, and glycoproteins, and (2) substances contained within the phospholipid classification, for example lecithin. The amine salts and the quaternary ammonium salts within the cationic group also comprise useful surfactants.

In some embodiments, the surfactant comprises a hydrophilic or amphiphilic surfactant such as polyoxyethylene (20) sorbitan monolaurate (TWEEN® 20) or polyvinyl alcohol that improves the distribution of the material in the polymeric carrier. In some embodiments, the surfactant comprises an amphiphilic surfactant if the second material is hydrophilic and the polymeric carrier is hydrophobic. In some embodiments, the surfactant is an anionic surfactant sodium bis(tridecyl) sulfosuccinate (Aerosol® TR-70). In some embodiments, the surfactant is sodium bis(tridecyl) sulfosuccinate, or sodium dodecyl sulfate (SDS).

### (c) Diagnostic Particle Size and Morphology

In some embodiments, the diagnostic particles may have a spherical shape. In some embodiments, the diagnostic particles may have cylindrical shape.

In some embodiments, the diagnostic particles may have a wide variety of non-spherical shapes. The non-spherical shaped diagnostic particles can be used to alter uptake by phagocytic cells and thereby clearance by the reticuloendothelial system. In some embodiments, the non-spherical diagnostic particles may be in the shape of rectangular disks, high aspect ratio rectangular disks, rods, high aspect ratio rods, worms, oblate ellipses, prolate ellipses, elliptical disks, UFOs, circular disks, barrels, bullets, pills, pulleys, bi-convex lenses, ribbons, ravioli, flat pill, bicones, diamond disks, emarginated disks, elongated hexagonal disks, tacos, wrinkled prolate ellipsoids, wrinkled oblate ellipsoids, or porous elliptical disks. Additional shapes beyond those are also within the scope of the definition for “non-spherical” shapes.

In some embodiments, the diagnostic particles have a PdI from about 0.05 to about 0.15, about 0.06 to about 0.14, about 0.07 to about 0.13, about 0.08 to about 0.12, or about 0.09 to about 0.11. In some embodiments, the diagnostic particles have a PdI of about 0.05, about 0.06, about 0.07, about 0.08, about 0.09, about 0.10, about 0.11, about 0.12, about 0.13, about 0.14, or about 0.15

In some embodiments, the diagnostic particle has a median size less than 1000 nm. In some embodiments, the median diagnostic particle size ranges from about 1 nm to about 1000 nm. In some embodiments, the median diagnostic particle size ranges from about 1 nm to about 500 nm. In some embodiments, the median diagnostic particle size ranges from about 1 nm to about 250 nm. In some embodiments, the median diagnostic particle size ranges from about 1 nm to about 150 nm. In some embodiments, the median diagnostic particle size ranges from about 1 nm to about 100 nm. In some embodiments, the median diagnostic particle size ranges from about 1 nm to about 50 nm. In some embodiments, the median diagnostic particle size ranges from about 1 nm to about 25 nm. In some embodiments, the median diagnostic particle size ranges from about 1 nm to about 10 nm. In some embodiments, the diagnostic particle has a median particle size selected from the group consisting of about 1 nm, about 5 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, about 200 nm, about 205 nm, about 210 nm, about 215 nm, about 220 nm, about 225 nm, about 230 nm, about 235 nm, about 240 nm, about 245 nm, about 250 nm, about 255 nm, about 260 nm, about 265 nm, about 270 nm, about 275 nm, about 280 nm, about 285 nm, about 290 nm, about 295 nm, about 300 nm, about 310 nm, about 320 nm, about 330 nm, about 340 nm, about 350 nm, about 360 nm, about 370 nm, about 380 nm, about 390 nm, about 400 nm, about 410 nm, about 420 nm, about 430 nm, about 440 nm, about 450 nm, about 460 nm, about 470 nm, about 480 nm, about 490 nm, about 500 nm, about 525 nm, about 550 nm, about 575 nm, about 600 nm, about 625 nm, about 650 nm, about 675 nm, about 700 nm, about 725 nm, about 750 nm, about 775 nm, about 800 nm, about 825 nm, about 850 nm, about 875 nm, about 900 nm, about 925 nm, about 950 nm, about 975 nm, and about 1000 nm. In some embodiments, the diagnostic particle has a median particle size of 500 nm. In some embodiments, the diagnostic particle has a median particle size of 250 nm. In some embodiments, the diagnostic particle has a median particle size of 750 nm.

In some embodiments, the diagnostic particles are microparticles having a median particle size equal or greater than 1000 nm (1 micron). In some embodiments, the diagnostic particles have a median particle size selected from the group consisting of about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 105 μm, about 110 μm, about 115 μm, about 120 μm, about 125 μm, about 130 μm, about 140 μm, about 145 μm, about 150 μm, about 155 μm, about 160 μm, about 165 μm, about 170 μm, about 175 μm, about 180 μm, about 185 μm, about 190 μm, about 195 μm, about 200 μm, about 205 μm, about 210 μm, about 215 μm, about 220 μm, about 225 μm, about 230 μm, about 235 μm, about 240 μm, about 245 μm, about 250 μm, about 255 μm, about 260 μm, about 265 μm, about 270 μm, about 275 μm, about 280 μm, about 285 μm, about 290 μm, about 295 μm, about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, and about 500 μm. In some embodiments, the diagnostic particle has a median particle size in a range from about 1 μm to about 500 μm. In some embodiments, the diagnostic particle has a median particle size in a range from about 1 μm to about 250 μm. In some embodiments, the diagnostic particle has a median particle size in a range from about 1 μm to about 100 μm. In some embodiments, the diagnostic particle has a median particle size in the range from about 1 μm to about 50 μm. In some embodiments, the diagnostic particle has a median particle size in a range from about 1 μm to about 25 μm. In some embodiments, the diagnostic particle has a median particle size in a range from about 1 μm to about 10 μm. In some embodiments, the diagnostic particle has a median particle size in a range from about 1 μm to about 6 μm. In some embodiments, the diagnostic particle has a median particle size from about 1 μm, about 2 μm, about 3 μm, about 4 μm, about 5 μm, or about 6 μm. In some embodiments, the diagnostic particle has a median particle size in the range from about 1 μm to about 4 μm.

In some embodiments, the diagnostic particle has a median particle size ni a range from about 100 nm to about 4 μm. In some embodiments, the diagnostic particles have a median particle size selected from the group consisting of about 100 nm, about 110 nm, about 120 nm, about 130 nm, about 140 nm, about 150 nm, about 160 nm, about 170 nm, about 180 nm, about 190 nm, about 200 nm, about 225 nm, about 250 nm, about 275 nm, about 300 nm, about 350 nm, about 400 nm, about 450 nm, about 500 nm, about 600 nm, about 700 nm, about 800 nm, about 900 nm, about 1000 nm, about 1.1 μm, about 1.2 μm, about 1.3 μm, about 1.4 μm, about 1.5 μm, about 1.6 μm, about 1.7 μm, about 1.8 μm, about 1.9 μm, about 2.0 μm, about 2.2 μm, about 2.4 μm, about 2.6 μm, about 2.8 μm, about 3.0 μm, about 3.2 μm, about 3.4 μm, about 3.6 μm, about 3.8 μm, about 4.0 μm, or any size between any two of these sizes.

In one embodiment, the zeta potential of the diagnostic particles is from about −60 mV to about 60 mV, from about −50 mV to about 50 mV, from about −30 mV to about 30 mV, from about −25 mV to about 25 mV, from about −20 mV to about 20 mV, from about −10 mV to about 10 mV, from about −10 mV to 5 mV, from about −5 mV to about 5 mV, or from about −2 mV to about 2 mV. In some embodiments, the zeta potential of the diagnostic particles is in a range selected from the group consisting of about −10 mV to about 10 mV, from about −5 mV to about 5 mV, and from about −2 mV to about 2 mV. In some embodiments, the diagnostic particle surface charge is neutral or near-neutral (i.e., zeta potential is from about −10 mV to about 10 mV).

In some embodiments, a spacer can link the β-lactamase inhibitor fragment and the β-lactam antibiotic fragment to each other. In some embodiments, the spacer links the two β-lactamase sensing components via an amide bond formed by NHS/EDC chemistry. In some embodiments, the spacer links the two β-lactamase sensing components via a disulfide (S—S) bond.

In some embodiments, the β-lactamase sensing components comprises β-lactamase inhibitor fragment -(amino-(spacer)x)y-β-lactam antibiotic fragment or β-lactam antibiotic fragment-(spacer)z-β-lactamase inhibitor fragment, wherein the spacer has from 2 to 50 atoms, x, y and z are integers from 5 to 15.

In some embodiments, the spacer joined the β-lactamase inhibitor fragment and the β-lactam antibiotic fragment via a degradable bond is selected from the group consisting of an ester bond, an amide bond, an imine bond, an acetal bond, a ketal bond, and combinations thereof.

In some embodiments, the spacer is selected from the group consisting of polyethylene glycol having 2-50 repeating units, ε-maleimidocaproic acid, para-aminobenzyloxy carbamater, and combinations thereof. In some embodiments, the spacer comprises polyamino acid having 2-30 amino acid residues. In some embodiments, the spacer comprises a linear polylysine, or polyglutamine.

In some embodiments, this disclosure provides a kit for detecting the presence of a β-lactamase, the kit comprises a solid support containing a dried form of a composition, wherein the composition comprises a detectable β-lactamase biosensor disclosed herein. In some embodiments, the solid support is a paper impregnated with the biosensor. In some embodiments, the solid support is a diagnostic particle encapsulating the biosensor.

In some embodiments, this disclosure provides a kit for detecting the presence of an erythromycin (macrolide) esterase, the kit comprises a solid support containing a dried form of a composition, wherein the composition comprises a detectable erythromycin (macrolide) esterase biosensor disclosed herein. In some embodiments, the solid support is a paper impregnated with the biosensor. In some embodiments, the solid support is a diagnostic particle encapsulating the biosensor.

In some embodiments, this disclosure provides a kit for detecting the presence of chloramphenicol hydrolase, the kit comprises a solid support containing a dried form of a composition, wherein the composition comprises a detectable chloramphenicol hydrolase biosensor disclosed herein. In some embodiments, the solid support is a paper impregnated with the biosensor. In some embodiments, the solid support is a diagnostic particle encapsulating the biosensor.

### (d) Diagnostic Particle Surface Modification

In some embodiments, the diagnostic particle surface further comprises a hydrophilic polymer that promotes prolonged blood circulation (known as “stealth”). Examples of the hydrophilic polymer include, but are not limited to, polyethylene glycol (PEG); PEG containing block copolymer; polyalkylene oxide, including polypropylene oxide, polybutylene oxide; block copolymer of PEG and polypropylene oxide; polyoxyethylene-polyoxypropylene block copolymer (Pluronic® F-68, F-127), polyxamer (polyethylene oxide block copolymer); hyperbranched polyglycerol; hyaluronic acid; or combinations thereof.

The presence of the hydrophilic polymer on the diagnostic particle surface can affect the zeta-potential of the diagnostic particle. In one embodiment, the zeta potential of the diagnostic particle is from about −60 mV to about 60 mV, from about −50 mV to about 50 mV, from about −30 mV to about 30 mV, from about −25 mV to about 25 mV, from about −20 mV to about 20 mV, from about −10 mV to about 10 mV, from about −10 mV to 5 mV, from about −5 mV to about 5 mV, or from about −2 mV to about 2 mV. In some embodiments, the zeta potential of the diagnostic particle is in a range selected from the group consisting of about −10 mV to about 10 mV, from about −5 mV to about 5 mV, and from about −2 mV to about 2 mV. In some embodiments, the diagnostic particle surface charge is neutral or near-neutral (i.e., zeta potential is from about −10 mV to about 10 mV).

In some embodiments, the hydrophilic polymer is a polyethylene glycol. In some embodiments, the hydrophilic polymer on the diagnostic particle surface is a polyethylene glycol having a number average molecular weight ranging from about 300 Da to about 100,000 Da. In some embodiments, the polyethylene glycol has a number average molecular weight selected from the group consisting of 300 Da, 600 Da, 1 kDa, 2 kDa, 3 kDa, 4 kDa, 6 kDa, 8 kDa, 10 kDa, 15 kDa, 20 kDa, 30 kDa, 50 kDa, 100 kDa, 200 kDa, and 500 kDa. Polyethylene glycol of any given molecular weight may vary in other characteristics such as length, density, and branching. In some embodiments, the diagnostic particle surface modifier is a PEG having a number average molecular weight ranging from 2000 Da to 80,000 Da. In some embodiments, the diagnostic particle surface modifier is a PEG having a number average molecular weight ranging from 2000 Da to 70,000 Da. In some embodiments, the diagnostic particle surface modifier is a PEG having a number average molecular weight ranging from 2000 Da to 60,000 Da. In some embodiments, the diagnostic particle surface modifier is a PEG having a number average molecular weight ranging from 2000 Da to 50,000 Da. In some embodiments, the diagnostic particle surface modifier is a PEG having a number average molecular weight ranging from 2000 Da to 40,000 Da. In some embodiments, the diagnostic particle surface modifier is a PEG having a number average molecular weight ranging from 2000 Da to 30,000 Da. In some embodiments, the diagnostic particle surface modifier is a PEG having a number average molecular weight ranging from 2000 Da to 20,000 Da. In some embodiments, the diagnostic particle surface modifier is a PEG having a number average molecular weight ranging from 2000 Da to 10,000 Da. In some embodiments, the diagnostic particle surface modifier is a PEG having a number average molecular weight ranging from 2000 Da to 9,000 Da. In some embodiments, the diagnostic particle surface modifier is a PEG having a number average molecular weight ranging from 2000 Da to 8,000 Da. In some embodiments, the diagnostic particle surface modifier is a PEG having a number average molecular weight ranging from 5000 Da to 10,000 Da. In some embodiments, the diagnostic particle surface modifier is a PEG having a number average molecular weight ranging from 7000 Da to 10,000 Da.

In some embodiments, the diagnostic particle surface modifier is a PEG having a number average molecular weight selected from the group consisting of 2000 Da, 3000 Da, 4000 Da, 5000 Da, 6000 Da, 7000 Da, 8000 Da, 9000 Da, 10,000 Da, 11,000 Da, 12,000 Da, 13,000 Da, 14,000 Da, 15,000 Da, 16,000 Da, 17,000 Da, 18,000 Da, 19,000 Da, 20,000 Da, 21,000 Da, 22,000 Da, 23,000 Da, 24,000 Da, 25,000 Da, 26,000 Da, 27,000 Da, 28,000 Da, 29,000 Da, 30,000 Da, 31,000 Da, 32,000 Da, 33,000 Da, 34,000 Da, 35,000 Da, 36,000 Da, 37,000 Da, 38,000 Da, 39,000 Da, 40,000 Da, 41,000 Da, 42,000 Da, 43,000 Da, 44,000 Da, 45,000 Da, 46,000 Da, 47,000 Da, 48,000 Da, 49,000 Da, 50,000 Da, 51,000 Da, 52,000 Da, 53,000 Da, 54,000 Da, 55,000 Da, 56,000 Da, 57,000 Da, 58,000 Da, 59,000 Da, 60,000 Da, 61,000 Da, 62,000 Da, 63,000 Da, 64,000 Da, 65,000 Da, 66,000 Da, 67,000 Da, 68,000 Da, 69,000 Da, 70,000 Da, 71,000 Da, 72,000 Da, 73,000 Da, 74,000 Da, 75,000 Da, 76,000 Da, 77,000 Da, 78,000 Da, 79,000 Da, 80,000 Da, 81,000 Da, 82,000 Da, 83,000 Da, 84,000 Da, 85,000 Da, 86,000 Da, 87,000 Da, 88,000 Da, 89,000 Da, 90,000 Da, 91,000 Da, 92,000 Da, 93,000 Da, 94,000 Da, 95,000 Da, 96,000 Da, 97,000 Da, 98,000 Da, 99,000 Da, and 100,000 Da.

In some embodiments, the amount of the hydrophilic polymer attached to the diagnostic particle surface is expressed as a percentage by the total weight of the uncoated diagnostic particle. In some embodiments, the weight ratio of the hydrophilic polymer to the uncoated diagnostic particle is at least 1/10,000, 1/7500, 1/5000, 1/4000, 1/3400, 1/2500, 1/2000, 1/1500, 1/1000, 1/750, 1/500, 1/250, 1/200, 1/150, 1/100, 1/75, 1/50, 1/25, 1/20, 1/5, 1/2, or 9/10 by the weight of the uncoated diagnostic particle. In some embodiments, the weight ratio of the hydrophilic polymer to the uncoated diagnostic particle is in a range from 1/10,000 to 9/10 by the weight of the uncoated diagnostic particle. In some embodiments, the hydrophilic polymer on the diagnostic particle surface has a weight percent by the weight of the uncoated diagnostic particle is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%. In some embodiments, the hydrophilic polymer covers at least 90% of the diagnostic particle surface area. In some embodiments, the hydrophilic polymer covers about 100% of the diagnostic particle surface area. In some embodiments, the hydrophilic polymer covers at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% of the diagnostic particle surface area.

### (e) Microbial Targeting Group

In some embodiments, the diagnostic particle disclosed herein can be readily engineered to carry out additional functions (e.g. localizing of diagnostic particles to the microbes). In some embodiments, the diagnostic particle further comprises a microbial-targeting group on the diagnostic particle surface. In some embodiments, the diagnostic particle surface is modified with microbial targeting moieties for active targeting. In some embodiments, a microbial-targeting group is selected from the group consisting of an antibody targeting a bacterial surface antigen; an antibody targeting a bacteria Toll Like Receptor (TLR); a cationic AMP; LPS binding compound; cell penetrating peptides, including apidaecin, tat, buforin, and magainin; and combinations thereof. In some embodiments, the microbial targeting group is a peptide, specifically a cyclic 9-amino acid peptide-CARGGLKSC (CARG). In some embodiments, the microbial targeting group is ubiquicidin (UBI29-41).

In some embodiments, the microbial-targeting group is a group targeting MSCRAMM (microbial surface components recognizing adhesive matrix molecules), GADPH (surface enzyme), LPXTG domain, Lipid A, β-barrel proteins commonly called outer membrane proteins (OMPs), or combinations thereof.

In some embodiments, the diagnostic particle surface is covalently conjugated with a positively charged moiety such as poly-lysine, chitosan etc. to localize the diagnostic particle to the negatively charged bacterial membrane.

In some embodiments, the diagnostic particle surface is labeled with a macrophage-targeting group selected from a group consisting of dextran, tuftsin, mannose, hyaluronate, and combinations thereof.

In some embodiments, the microbial-targeting group is a ligand targeting pneumococcal surface protein A (PspA), putative proteinase maturation protein A (PpmA), pneumococcal surface adhesin A (PsaA), surface protein G, known as adhesin SasG, staphylococcal protein A (SpA), clumping factor B (ClfB), clumping factor A (clfA), collagen adhesin (CNA), SesL, SesB, SesC, SesK, SesM, Bam A (OMP), adhesin protein (intimin), Hsp90, FimH, OmpA, IROMPS (Iron Regulated Outer Membrane Proteins), M proteins (LPXTG conserved motif in strep), PGK (surface enzyme), TPI (surface enzyme), PGM (surface enzyme), C5a peptidase, SclA (Scl1), GRAB, pullulanase, Esp, Oprl (outer membrane protein I), PilY1, or combinations thereof.

In some embodiments, the microbial-targeting group is selected from the group consisting of a microbial-binding portion of C-type lectins, Col-like lectins, ficolins, receptor-based lectins, lectins from the shrimp Marsupenaeus japonicas, non-C-type lectins, a lipopolysaccharide (LPS)-binding proteins, endotoxin-binding proteins, mannan-binding lectin (MBL), surfactant protein A, surfactant protein D, collectin 11, L-ficolin, ficolin A, DC-SIGN, DC-SIGNR, SIGNR1, macrophage mannose receptor 1, dectin-1, dectin-2, lectin A, lectin B, lectin C, wheat germ agglutinin, CD 14, MD2, lipopolysaccharide-binding protein (LBP), limulus anti-LPS factor (LAL-F), mammalian peptidoglycan recognition protein-1 (PGRP-1), PGRP-2, PGRP-3, PGRP-4, and combinations thereof. In some embodiments, the microbe-targeting group is a LPS binding protein. In some embodiments, the microbe-targeting group is an endotoxin-binding protein.

In some embodiments, AMP is the targeting group. AMP binds to negatively charged bacterial cell membranes via electrostatic interactions, disrupting their function, and resulting in the death of these prokaryotes.

In some embodiments, the microbial targeting group is a cyclic peptide antibiotic vancomycin and/or polymyxin (e.g., polymyxin B, polymyxin E).

In some embodiments, the microbial-targeting group is chemically conjugated to the surface of the diagnostic particle by EDC-NHS chemistry where the primary amine groups of the targeting antibody/peptide are conjugated to the reactive —COOH groups on the diagnostic particle surface, such as those from gelatin, collagen, or protein carrier.

In some embodiments, the diagnostic particle surface is labeled with RGD sequences or a positively charged polymer, such as poly-lysine, chitosan etc., via covalent bonding to target the diagnostic particle to the negatively charged bacteria membrane.

In some embodiments, the microbial-targeting group is the TAT (YGRKKRRQRRR) peptide that is covalently bound onto the diagnostic particle surface. The TAT peptide is the shortest amino-acid sequence required for membrane translocation. The TAT peptide was found in the transcriptional activator TAT protein of the human immunodeficiency virus type-1 (HIV-1).

In some embodiments, the density of display of the targeting group on the diagnostic particle surface is from about 1 ligand/nm² to about 50 ligands/nm². In some embodiments, the density of display of the targeting group (ligand) on the diagnostic particle surface is selected from the group consisting of about 1 ligand/nm², 2 ligands/nm², about 3 ligands/nm², about 4 ligands/nm², about 5 ligands/nm², about 6 ligands/nm², about 7 ligands/nm², about 8 ligands/nm², about 9 ligands/nm², about 10 ligands/nm², about 11 ligands/nm², about 12 ligands/nm², about 13 ligands/nm², about 14 ligands/nm², about 15 ligands/nm², about 16 ligands/nm², about 17 ligands/nm², about 18 ligands/nm², about 19 ligands/nm², about 20 ligands/nm², about 21 ligands/nm², about 22 ligands/nm², about 23 ligands/nm², about 24 ligands/nm², about 25 ligands/nm², about 26 ligands/nm², about 27 ligands/nm², about 28 ligands/nm², about 29 ligands/nm², about 30 ligands/nm², about 31 ligands/nm², about 32 ligands/nm², about 33 ligands/nm², about 34 ligands/nm², about 35 ligands/nm², about 36 ligands/nm², about 37 ligands/nm², about 38 ligands/nm², about 39 ligands/nm², about 40 ligands/nm², about 41 ligands/nm², about 42 ligands/nm², about 43 ligands/nm², about 44 ligands/nm², about 45 ligands/nm², about 46 ligands/nm², about 47 ligands/nm², about 48 ligands/nm², about 49 ligands/nm², about 50 ligands/nm², about 60 ligands/nm², about 70 ligands/nm², about 80 ligands/nm², about 90 ligands/nm², about 100 ligands/nm², about 110 ligands/nm², about 120 ligands/nm², about 130 ligands/nm², about 140 ligands/nm², about 150 ligands/nm², about 160 ligands/nm², about 170 ligands/nm², about 180 ligands/nm², about 190 ligands/nm², and about 200 ligands/nm² of the diagnostic particle surface area.

### Theragnostic Particles

In an embodiment, this disclosure provides a theragnostic particle comprising the diagnostic particles as disclosed herein and a second material interacting with an exogenous energy source, wherein the theragnostic particle can be used to detect, localize, and destroy microbes using the exogenous source. If the theragnostic particle produces a detectable change of optical response that indicates the presence of drug-resistant microbes (e.g., a color change from colorless to a bright color (e.g., blue or violet), or from bright color to colorless), then an exogenous energy source is applied to the theragnostic particle to induce an energy-to-heat conversion, whereby localized hyperthermia is induced to quickly kill the microbes. There is no known resistance mechanism for such a remotely-triggered hyperthermia treatment on drug resistant microbes that would be very useful for the treatment of multidrug resistant microbes.

In some embodiments, the theragnostic particle further passes the Efficacy Determination Protocol.

In some embodiments, theragnostic particle further passes the Thermal Cytotoxicity Toxicity Test.

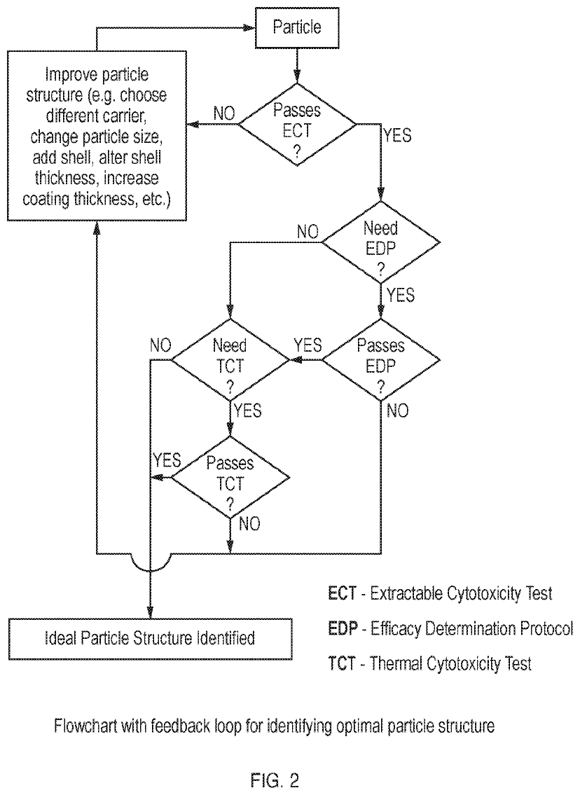

Efficacy Determination Protocol in conjunction with the Extractable Cytotoxicity Test and/or Thermal Cytotoxicity Test will provide feedback (feedback loop protocol) to optimize the particle structure such that the biosensors, the second material and/or anticancer agent can be protected from the degradation by body chemicals. The Extractable Cytotoxicity Test is conducted according to the protocols set forth below (See FIG. 2). The particle structure characteristics (e.g. carrier material selection, particle size, morphology, particle surface modification etc.) and the laser irradiation method characteristics (e.g. laser wavelength, pulse duration and energy efficiency) are optimized sequentially based on the structure-property relationship feedbacks provides from the tests in the flow chart of FIG. 2 including Extractable Cytotoxicity Test, Efficacy Determination Test and/or Thermal Cytotoxicity Test. The ideal theragnostic particles possess the characteristics of high energy-to-heat conversion efficiency, stability (including thermal stability), and low collateral damage.

In some embodiments, the biosensor in the theragnostic particle is a compound selected from those disclosed in Table 1.

In some embodiments, the biosensor and the second material are encapsulated within the theragnostic particle. In some embodiments, the biosensor and/or the second material are bound to the interior of the theragnostic particle via a covalent bond. In some embodiments, the biosensor is bound to the exterior surface of the theragnostic particle via a covalent bond, and the second material is encapsulated within the theragnostic particle. In some embodiments, the formation of the covalent bond is via NHS/EDC chemistry, bisulfide bond, or click chemistry.

In some embodiments, the theragnostic particle is porous and the pores of the particle are plugged with a protein or peptide degradable by enzymes secreted by the microbes. When the protein/peptide plugged theragnostic particle is contacted with the liquid medium of the testing sample, the enzyme secreted by the microbes cause degradation of the protein/peptide plug such that the theragnostic particle becomes permeable to the liquid medium of the testing sample. These particles are useful for detecting microbes prior to the thermal therapy.

In some embodiments, the theragnostic particle is porous and the pores of the particle are plugged with a lipid. When the theragnostic particles are activated by an exogenous source to induce localized hyperthermia, the lipid plug melts and causes the theragnostic particle to become permeable to the liquid medium of the testing sample. These particles are useful for detecting residual microbes after thermal therapy.

In some embodiments, theragnostic particles with different designs can be applied independently in wound care and wound healing treatments. In some embodiments, two or more different populations of the distinct theragnostic particles as disclosed herein can be applied concurrently or sequentially in wound care and wound healing treatments.

In some embodiments, the exogenous source is selected from the group consisting of an electromagnetic radiation, an electrical field, a microwave, a radio wave, an ultrasound, a magnetic field, or combinations thereof.

The theragnostic particle based combination therapies for treatment of microbial infection provides improved therapeutic index as compared to standalone antimicrobial chemotherapies or PTT/PDT alone.

a. Second Material

In some embodiments, the second material in the theragnostic particles interacts with the exogenous energy source to produce heat that performs a function, like inducing cytotoxicity to the microbes by raising the temperature to above normal body temperature without causing collateral damages to the host cells.

In some embodiments, the exogenous energy source is electromagnetic radiation (NIR, LED), microwaves, radio waves, and sound waves, electrical or magnetic field.

In some embodiments, the exogenous energy source may be electromagnetic radiation (EMR). In some embodiments, the second material interacting with the exogenous energy source does not have significant optical absorption in the visible region of EMR. In some embodiments, the second material interacting with the exogenous energy source comprises a dye capable of absorbing EMR and converting the energy into heat (photothermal conversion).

In some embodiments, the second material interacting with the exogenous energy source does not have significant optical absorption in the visible region of EMR. In some embodiments, the second material interacting with the exogenous source comprises a dye capable of absorbing EMR and converting the energy into heat (photothermal conversion). In some embodiments, the absorption spectrum range of the second material does not overlap with the spectrum wavelength of the first material, for example, the first material exhibits a visible color of red (625-740 nm), cyan (485-500 nm), yellow (565-590 nm), magenta, violet (380-450 nm) or blue (450-485 nm). The spectral various color include violet (380-450 nm), blue (450-485 nm), cyan (485-500 nm), green (500-565 nm), yellow (565-590 nm), orange (590-625 nm) and red (625-740 nm).

In some embodiments, the spectroscopic probe has absorption in the visible range (400 nm to 750 nm) and the second material interacting with the exogenous source has significant absorption in the near infrared spectrum region (NIR) (750 nm to 1500 nm). In some embodiments, the spectroscopic probe has absorption in the visible range (400 nm to 750 nm) and the second material has significant absorption in the near infrared spectrum region (NIR) (400 nm to 750 nm). In some embodiments, the second material has significant absorption of LED light having a wavelength of 750 nm to 1050 nm. In some embodiments, the second material interacting with the exogenous source has significant absorption of LED light having a wavelength of 750 nm to 940 nm (infrared LEDs or IR LEDs). In some embodiments, the LED light source is a LE7-IR™ instrument by Image Engineer having 480 LED channels including 11 IR channels that create different spectra not only in the visible but also in the near infrared spectrum up to 1050 nm.

In some embodiments, the second material interacting with the exogenous source has significant absorption at NIR wavelengths in the range from 750 nm to 1500 nm. In some embodiments, the second material interacting with the exogenous source has significant absorption at NIR wavelengths in the range from 750 nm to 1400 nm. In some embodiments, the second material interacting with the exogenous source has significant absorption at NIR wavelengths in the range from 750 nm to 1300 nm. In some embodiments, the second material interacting with the exogenous source has significant absorption at NIR wavelengths in the range from 750 nm to 900 nm. In some embodiments, the second material interacting with the exogenous source has significant absorption at NIR wavelengths in the range from 750 nm to 950 nm. In some embodiments, the second material interacting with the exogenous source has significant absorption at NIR wavelengths in the range from 800 nm to 1100 nm. In some embodiments, the second material interacting with the exogenous source has significant absorption at NIR wavelengths in the range from 750 nm to 850 nm. In some embodiments, the second material interacting with the exogenous source has significant absorption at NIR wavelengths in the range from 1000 nm to 1400 nm. In some embodiments, the second material interacting with the exogenous source has significant absorption at NIR wavelengths in the range from 1000 nm to 1300 nm. In some embodiments, the second material interacting with the exogenous source has significant absorption at NIR wavelengths in the range from 1000 nm to 1100 nm.

In some embodiments, the second material interacting with the exogenous source has significant absorption at a wavelength selected from the group consisting of 750 nm, 751 nm, 752 nm, 753 nm, 754 nm, 755 nm, 756 nm, 757 nm, 756 nm, 756 nm, 758 nm, 759 nm, 760 nm, 761 nm, 762 nm, 763 nm, 764 nm, 765 nm, 766 nm, 767 nm, 768 nm, 769 nm, 770 nm, 771 nm, 772 nm, 773 nm, 774 nm, 775 nm, 776 nm, 777 nm, 778 nm, 779 nm, 780 nm, 781 nm, 782 nm, 783 nm, 784 nm, 785 nm, 786 nm, 787 nm, 789 nm, 790 nm, 791 nm, 792 nm, 793 nm, 794 nm, 795 nm, 796 nm, 797 nm, 798 nm, 799 nm, 800 nm, 801 nm, 802 nm, 803 nm, 804 nm, 805 nm, 806 nm, 807 nm, 808 nm, 809 nm, 810 nm, 811 nm, 812 nm, 813 nm, 814 nm, 815 nm, 816 nm, 817 nm, 818 nm, 819 nm, 820 nm, 821 nm, 822 nm, 823 nm, 824 nm, 825 nm, 826 nm, 827 nm, 828 nm, 829 nm, 830 nm, 831 nm, 832 nm, 833 nm, 834 nm, 835 nm, 836 nm, 837 nm, 838 nm, 839 nm, 840 nm, 841 nm, 842 nm, 843 nm, 844 nm, 845 nm, 846 nm, 847 nm, 848 nm, 849 nm, 850 nm, 851 nm, 852 nm, 853 nm, 854 nm, 855 nm, 856 nm, 857 nm, 858 nm, 859 nm, 860 nm, 861 nm, 862 nm, 863 nm, 864 nm, 865 nm, 866 nm, 867 nm, 868 nm, 869 nm, 870 nm, 871 nm, 872 nm, 873 nm, 874 nm, 875 nm, 876 nm, 877 nm, 878 nm, 879 nm, 880 nm, 881 nm, 882 nm, 883 nm, 884 nm, 885 nm, 886 nm, 887 nm, 888 nm, 889 nm, 890 nm, 891 nm, 892 nm, 893 nm, 894 nm, 895 nm, 896 nm, 897 nm, 898 nm, 899 nm, 900 nm, 901 nm, 902 nm, 903 nm, 904 nm, 905 nm, 906 nm, 907 nm, 908 nm, 909 nm, 910 nm, 911 nm, 912 nm, 913 nm, 914 nm, 915 nm, 916 nm, 917 nm, 918 nm, 919 nm, 920 nm, 921 nm, 922 nm, 923 nm, 924 nm, 925 nm, 926 nm, 927 nm, 928 nm, 929 nm, 930 nm, 931 nm, 932 nm, 933 nm, 934 nm, 935 nm, 936 nm, 937 nm, 938 nm, 939 nm, 940 nm, 941 nm, 942 nm, 943 nm, 944 nm, 945 nm, 946 nm, 947 nm, 948 nm, 949 nm, 950 nm, 951 nm, 952 nm, 953 nm, 954 nm, 955 nm, 956 nm, 957 nm, 958 nm, 959 nm, 960 nm, 961 nm, 962 nm, 963 nm, 964 nm, 965 nm, 966 nm, 967 nm, 968 nm, 969 nm, 970 nm, 971 nm, 972 nm, 973 nm, 974 nm, 975 nm, 976 nm, 977 nm, 978 nm, 979 nm, 980 nm, 981 nm, 982 n, 983 nm, 984 nm, 985 nm, 986 nm, 987 nm, 988 nm, 989 nm, 990 nm, 991 nm, 992 nm, 993 nm, 994 nm, 995 nm, 996 nm, 997 nm, 998 nm, 999 nm, 1000 nm, 1001 nm, 1002 nm, 1003 nm, 1004 nm, 1005 nm, 1006 nm, 1007 nm, 1008 nm, 1009 nm, 1010 nm, 1011 nm, 1012 nm, 1013 nm, 1014 nm, 1015 nm, 1016 nm, 1017 nm, 1018 nm, 1019 nm, 1020 nm, 1021 nm, 1022 nm, 1023 nm, 1024 nm, 1025 nm, 1026 nm, 1027 nm, 1028 nm, 1029 nm, 1030 nm, 1031 nm, 1032 nm, 1033 nm, 1034 nm, 1035 nm, 1036 nm, 1037 nm, 1038 nm, 1039 nm, 1040 nm, 1041 nm, 1042 nm, 1043 nm, 1044 nm, 1045 nm, 1046 nm, 1047 nm, 1048 nm, 1049 nm, 1050 nm, 1051 nm, 1052 nm, 1053 nm, 1054 nm, 1055 nm, 1056 nm, 1057 nm, 1058 nm, 1059 nm, 1060 nm, 1061 nm, 1062 nm, 1063 nm, 1064 nm, 1065 nm, 1066 nm, 1067 nm, 1068 nm, 1069 nm, 1070 nm, 1071 nm, 1072 nm, 1073 nm, 1074 nm, 1075 nm, 1076 nm, 1077 nm, 1078 nm, 1079 nm, 1080 nm, 1081 nm, 1082 nm, 1083 nm, 1084 nm, 1085 nm, 1086 nm, 1087 nm, 1088 nm, 1089 nm, 1090 nm, 1091 nm, 1092 nm, 1093 nm, 1094 nm, 1095 nm, 1096 nm, 1097 nm, 1098 nm, 1099 nm, and 1100 nm. In some embodiments, the second material interacting with the exogenous source has significant absorption at a wavelength ranges from 630 nm to 750 nm. In some embodiments, the second material interacting with the exogenous source has significant absorption at a wavelength selected from the group consisting of 766 nm, 777 nm, 780 nm, 783 nm, 785 nm, 800 nm, 805 nm, 808 nm, 810 nm, 820 nm, 825 nm, 900 nm, 948 nm, 950 nm, 960 nm, 980 nm, 1000 nm, 1064 nm, 1065 nm, 1070 nm, 1071 nm, 1073 nm, 1098 nm, and 1100 nm. In some embodiments, the second material interacting with the exogenous source has significant absorption at 1064 nm wavelength. In some embodiments, the second material interacting with the exogenous source has significant absorption at 1064 nm wavelength. In some embodiments, the second material interacting with the exogenous source has significant absorption at 805 nm wavelength. In some embodiments, the second material interacting with the exogenous source has significant absorption at 808 nm wavelength.

In some embodiments, the second material interacting with the exogenous source has significant absorption of photonic energy in the visible range. In some embodiments, the second material absorbs light at a wavelength ranging from 400 nm to 750 nm. In some embodiments, the material absorbs light at a wavelength selected from the group consisting of 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, and 750 nm.

In some embodiments, the second material interacting with exogenous source is an IR absorbing material. In some embodiments, the IR absorbing material comprises organic dyes or inorganic pigments. In some embodiments, the IR absorbing material is an IR dye. In some embodiments, the IR dye is an aminium and/or di-imonium dye having hexafluoroantimonate, tetrafluoroborate, or hexafluorophosphate as counterion. In some embodiments, an IR absorbing material may be utilized (e.g., N,N,N,N-tetrakis(4-dibutylaminophenyl)-p-benzoquinone bis(iminium hexafluoroantimonate, commercially available as ADS1065 from American Dye Source, Inc.). The absorption spectrum of ADS1065 dye has a maximum absorption at about 1065 nm, with low absorption in the visible region of the spectrum.

In some embodiments, the second material is an IR absorbing dye such as those Epolight™ aminium dyes made by Epolin Inc. of Newark, N.J. In some embodiments, the IR absorbing dye is an di-imonium dye (also aminium dye) having formula (I)

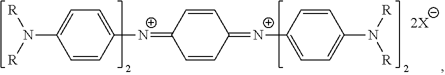

wherein R is a substituted or unsubstituted aryl, heteroaryl, C1-C8 alkyl, C1-C8 alkenyl, or C1-C8 alkynyl group, wherein the C1-C8 alkyl, C1-C8 alkenyl, or C1-C8 alkynyl group may be linear or branched, wherein X— is a counterion selected from the group consisting of hexafluoroarsenate (AsF₆⁻), hexafluoroantimonate (SbF₆⁻), hexafluorophosphate (PF₆⁻), (C₆F₅)₄B⁻, tetrafluoroborate (BF₄⁻), and combinations thereof. In some embodiments, the di-imonium dye of formula (I) has hexafluorophosphate as counterion. In some embodiments, the di-imonium dye of formula (I) has hexafluoroantimonate as counterion. In some embodiments, the di-imonium dye of formula (I) has tetrakis (perfluorophenyl) borate as counterion. In some embodiments, the IR absorbing dye is a tetrakis aminium dye, with a counterion containing metal element such as boron or antimony. In some embodiments, the tetrakis aminium dye compounds have formula (II)

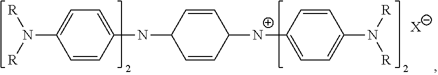

wherein R is a substituted or unsubstituted aryl, heteroaryl, C1-C8 alkyl, C1-C8 alkenyl, or C1-C8 alkynyl group, wherein the C1-C8 alkyl, C1-C8 alkenyl, or C1-C8 alkynyl group may be linear or branched, wherein X— is a counterion selected from the group consisting of hexafluoroarsenate (AsF₆⁻), hexafluoroantimonate (SbF₆⁻), hexafluorophosphate (PF₆⁻), (C₆F₅)₄B—, tetrafluoroborate (BF₄⁻), and combinations thereof. In some embodiments, the tetrakis aminium dyes are narrow band absorbers including commercially available dyes sold under the trademark names Epolight™ 1117 (tetrakis aminium dye having hexafluorophosphate counterion, peak absorption, 1071 nm), Epolight™ 1151 (tetrakis aminium dye, peak absorption, 1070 nm), or Epolight™ 1178 (tetrakis aminium dye, peak absorption, 1073 nm). Epolight™ 1151 (tetrakis aminium dye, peak absorption, 1070 nm), or Epolight™ 1178 (tetrakis aminium dye, peak absorption, 1073 nm). In some embodiments, the tetrakis aminium dyes are broad band absorbers including commercially available dyes sold under the trademark names Epolight™ 1175 (tetrakis aminium dye, peak absorption, 948 nm), Epolight™ 1125 (tetrakis aminium dye, peak absorption, 950 nm), and Epolight™ 1130 (tetrakis aminium dye, peak absorption, 960 nm).

In some embodiments, the tetrakis aminium dye is Epolight™ 1178 made by Epolin. In some embodiments, the IR absorbing material is a tetrakis aminium dye, which has minimal visible color. In some embodiments, the tetrakis aminium dye is Epolight™ 1117 (molecular weight, 1211 Da, peak absorption 1098 nm).

Other suitable aminium and/or di-imonium dyes suitable for the invention in this disclosure may be found in U.S. Pat. Nos. 3,440,257, 3,484,467, 3,400,156, 5,686,639, all of which are hereby fully incorporated by reference herein in their entirety. Additional counterions for the aminium and/or di-imonium dyes may be found in U.S. Pat. No. 7,498,123, which is hereby fully incorporated by reference herein in its entirety.

In some embodiments, the second material is an IR absorbing agent selected from the group consisting of 1-butyl-2-(2-[3-[2-(1-butyl-1H-benzo[cd]indol-2-ylidene)-ethylidene]-2-chloro-cyclohex-1-enyl]-vinyl)-benzo[cd]indolium tetrafluoroborate, 1-butyl-2-(2-[3-[2-(1-butyl-1H-benzo[cd]indol-2-ylidene)-ethylidene]-2-phenyl-cyclopent-1-enyl]-vinyl)-benzo[cd]indolium tetrafluoroborate, 1-butyl-2-(2-[3-[2-(1-butyl-1H-benzo[cd]indol-2-ylidene)-ethylidene]-2-phenyl-cyclohex-1-enyl]-vinyl)-benzo[cd]indolium tetrafluoroborate, 1-butyl-2-(2-[3-[2-(1-butyl-1H-benzo[cd]indol-2-ylidene)-ethylidene]-2-diphenylamino-cyclopent-1-enyl]vinyl)-benzo[cd]indolium tetrafluoroborate, 1-Butyl-2-[2-[3-[(1-butyl-6-chlorobenz[cd]indol-2(1H)-ylidene)ethylidene]-2-chloro-1-cyclohexen-1-yl]ethenyl]-6-chlorobenz[cd]indolium tetrafluoroborate (IR 1048), 1-butyl-2-[2-[3-[(1-butyl-6-chlorobenz[cd]indol-2(1H)-ylidene)ethylidene]-2-chloro-5-methyl-1-cyclohexen-1-yl]ethenyl]-6-chlorobenz[cd]indolium tetrafluoroborate (Lumogen™ IR 1050 by BASF), 4-[2-[2-chloro-3-[(2,6-diphenyl-4H-thiopyran-4-ylidene)ethylidene]-1-cyclohexen-1-yl]ethenyl]-2,6-diphenylthiopyrylium tetrafluoroborate (IR 1061), dimethyl{4-[1,7,7-tris(4-dimethylaminophenyl)-2,4,6-heptatrienylidene]-2,5-cyclohexadien-1-ylidene}ammonium perchlorate (IR 895), 2-[2-[2-chloro-3-[[1,3-dihydro-1,1-dimethyl-3-(4-sulfobutyl)-2H-benzo[e]indol-2-ylidene]-ethylidene]-1-cyclohexen-1-yl]-ethenyl]-1,1-dimethyl-3-(4-sulfobutyl)-1H-benzo[e]indolium hydroxide inner salt, sodium salt (IR820, new ICG dye), heptamethine cyanine (IR825), heptamethine cyanine (IR780), 4-hydroxybenzoic acid appended heptamethine cyanine, amine functionalized heptamethine cyanine, hemicyanine rhodamine, cryptocyanine, diketopyrrolopyrole, diketopyrrolopyrole-croconaine, 1,3-bis(5-(ethyl(2-(prop-2-yn-1-yloxy)ethyl)amino)thiophen-2-yl)-4,5-dioxocyclopent-2-en-1-ylium-2-olate (diaminothiophene-croconaine dye), potassium 1,1′-((2-oxido-4,5-dioxocyclopent-2-en-1-ylium-1,3-diyl)bis(thiophene-5,2-diyl))bis(piperidine-4-carboxylate) (dipiperidylthiophene-croconaine dye), indocyanine green (ICG), Cyanine 7 (Cy7®), and combinations thereof.

In some embodiments, the second material is selected from the group consisting of phthalocyanines, naphthalocyanines, and combinations thereof. In some embodiments, the second material is selected from the group consisting of a tetrakis aminium dye, a cyanine dye, a squaraine dye, a squarylium dye, iron oxide, a gold nanostructure, a zinc iron phosphate pigment, and combinations thereof. In some embodiments, the second material is a squaraine dye. In some embodiments, the second material is a squarylium dye. In some embodiments, the second material is a tetrakis aminium dye. In some embodiments, the second material is a cyanine dye.

In some embodiments, the second material is indocyanine green (ICG). After the ICG nanoparticles are irradiated with pulsed laser light, excited ICG dye produces singlet oxygen species (ROS) in the presence of cellular water, of which ROS is lethal for the microbes.

In some embodiments, the squarylium dye is a benzopyrylium squarylium dyes having

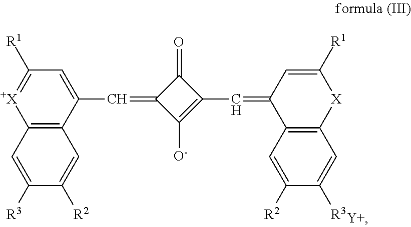

wherein each A is independently O, S, Se; Y+ is a counterion selected from the group consisting of hexafluoroarsenate (AsF₆⁻), hexafluoroantimonate (SbF₆⁻), hexafluorophosphate (PF₆⁻), (C₆F₅)₄B—, and tetrafluoroborate (BF₄⁻); each R1 is a non-aromatic organic substituent, each R2=H or OR3, R3=cycloalkyl, alkenyl, acyl, silyl; each R3=—NR4R5, each R4, R5 is independently H, C1-8 alkyl. In some embodiments, the squarylium dye of formula (III) is a compound when R1=—CMe3, R2=OCHMeEt, X=O with a strong absorption at 788 nm. In some embodiments, the squarylium dye of formula (III) is a compound when R1=—CMe3, R2=H, R3=—NEt2, X=O with a strong absorption at 808 nm (IR 193 dye).

In some embodiments, the second material comprises cyanine dyes selected from the group consisting indocyanine dye (ICG), 2-[2-[2-chloro-3-[[1,3-dihydro-1,1-dimethyl-3-(4-sulfobutyl)-2H-benzo[e]indol-2-ylidene]-ethylidene]-1-cyclohexen-1-yl]-ethenyl]-1,1-dimethyl-3-(4-sulfobutyl)-1H-benzo[e]indolium hydroxide inner salt, sodium salt (IR820, new ICG dye), heptamethine cyanine (IR825), heptamethine cyanine (IR780), and combinations thereof. In some embodiments, the second material may include indocyanine green (ICG) or new ICG IR 820 dye.

In some embodiments, the second material may include a squarylium dye selected from the group consisting of (IR 193 dye), 1,3-bis[[2-(1,1-dimethylethyl)-4H-1-benzopyran-4-ylidene]methyl]-2,4-dihydroxy-cyclobutenediylium salt, 1,3-dihydroxy-2,4-bis[(2-phenyl-4H-1-benzopyran-4-ylidene)methyl]-cyclobutenediylium salt, 1,3-bis[[2-(1,1-dimethylethyl)-6-methyl-4H-1-benzopyran-4-ylidene]methyl]-2,4-dihydroxy-cyclobutenediylium salt, 1,3-bis[[2-(1,1-dimethylethyl)-7-hydroxy-4H-1-benzopyran-4-ylidene]methyl]-2,4-dihydroxy-cyclobutenediylium salt, 1,3-bis[[2-(1,1-dimethylethyl)-6-(1-methylethyl)-4H-1-benzopyran-4-ylidene]methyl]-2,4-dihydroxy-cyclobutenediylium salt, 1,3-dihydroxy-2,4-bis[1-(2-phenyl-4H-1-benzopyran-4-ylidene)ethyl]-cyclobutenediylium salt, 1,3-dihydroxy-2,4-bis[(2-phenyl-4H-naphtho[1,2-b]pyran-4-ylidene)methyl]-cyclobutenediylium salt, 1,3-dihydroxy-2,4-bis[[6-(1-methylethyl)-2-phenyl-4H-1-benzopyran-4-ylidene]methyl]-cyclobutenediylium salt, 1,3-bis[[6-(1,1-dimethylethyl)-2-phenyl-4H-1-benzopyran-4-ylidene]methyl]-2,4-dihydroxy-cyclobutenediylium salt, 1,3-bis[(2-cyclohexyl-7-methoxy-4H-1-benzopyran-4-ylidene)methyl]-2,4-dihydroxy-cyclobutenediylium salt, 1,3-bis[[2-(1,1-dimethylethyl)-6-(1-methylpropoxy)-4H-1-benzopyran-4-ylidene]methyl]-2,4-dihydroxy-cyclobutenediylium salt, 1,3-bis[[8-chloro-2-(1,1-dimethylethyl)-6-(1-methylethyl)-4H-1-benzopyran-4-ylidene]methyl]-2,4-dihydroxy-cyclobutenediylium salt, 1,3-bis[[7-(dimethylamino)-2-(1,1-dimethylethyl)-4H-1-benzopyran-4-ylidene]methyl]-2,4-dihydroxy-cyclobutenediylium salt, 1-[[7-(diethylamino)-2-(1,1-dimethylethyl)-4H-1-benzopyran-4-ylidene]methyl]-3-[[7-(dimethylamino)-2-(1,1-dimethylethyl)-4H-1-benzopyran-4-ylidene]methyl]-2,4-dihydroxy-cyclobutenediylium salt, 1,3-bis[[7-(diethylamino)-2-(1,1-dimethylethyl)-4H-1-benzopyran-4-ylidene]methyl]-2,4-dihydroxy-cyclobutenediylium salt, 1,3-bis[1-[7-(diethylamino)-2-(1,1-dimethylethyl)-4H-1-benzopyran-4-ylidene]ethyl]-2,4-dihydroxy-cyclobutenediylium salt, 1-[[7-(diethylamino)-2-(1,1-dimethylethyl)-4H-1-benzopyran-4-ylidene]methyl]-3-[[2-(1,1-dimethylethyl)-7-(2-ethylbutoxy)-4H-1-benzopyran-4-ylidene]methyl]-2,4-dihydroxy-cyclobutenediylium salt, 1,3-bis[[2-cyclohexyl-7-(diethylamino)-4H-1-benzopyran-4-ylidene]methyl]-2,4-dihydroxy-cyclobutenediylium salt, 1,3-bis[[2-(1,1-dimethylethyl)-7-(1-piperidinyl)-4H-1-benzopyran-4-ylidene]methyl]-2,4-dihydroxy-cyclobutenediylium salt, 1,3-bis[[2-(1,1-dimethylethyl)-7-(hexahydro-1H-azepin-1-yl)-4H-1-benzopyran-4-ylidene]methyl]-2,4-dihydroxy-cyclobutenediylium salt, 1,3-bis[[2-(1,1-dimethylethyl)-7-(4-morpholinyl)-4H-1-benzopyran-4-ylidene]methyl]-2,4-dihydroxy-cyclobutenediylium salt, 1,3-bis[[11-(1,1-dimethylethyl)-2,3,6,7-tetrahydro-1H,5H,9H-[1]benzopyrano[6,7,8-ij]quinolizin-9-ylidene]methyl]-2,4-dihydroxy-cyclobutenediylium salt, 1,3-bis[[2-(1,1-dimethylethyl)-6-(4-morpholinyl)-4H-1-benzopyran-4-ylidene]methyl]-2,4-dihydroxy-cyclobutenediylium salt, 1,3-bis[[2-bicyclo[2.2.1]hept-5-en-2-yl-7-(diethylamino)-4H-1-benzopyran-4-ylidene]methyl]-2,4-dihydroxy-cyclobutenediylium salt, 1,3-bis[[7-(2,3-dihydro-1Hindol-1-yl)-2-(1,1-dimethylethyl)-4H-1-benzopyran-4-ylidene]methyl]-2,4-dihydroxy-cyclobutenediylium salt, 1,3-bis[[7-(diethylamino)-2-[(1R,5S)-6,6-dimethylbicyclo[3.1.1]hept-2-en-2-yl]-4H-1-benzopyran-4-ylidene]methyl]-2,4-dihydroxy-cyclobutenediylium salt, 1,3-bis[[7-(diethylamino)-2-(6,6-dimethylbicyclo[3.1.1]hept-2-en-3-yl)-4H-1-benzopyran-4-ylidene]methyl]-2,4-dihydroxy-cyclobutenediylium salt, 1,3-dihydroxy-2,4-bis[[7-(4-morpholinyl)-2-tricyclo[3.3.1.13,7]dec-1-yl-4H-1-benzopyran-4-ylidene]methyl]-cyclobutenediylium salt, 2,4-bis[[7-(diethylamino)-2-(1,1-dimethylethyl)-4H-1-benzopyran-4-ylidene]methyl]-1,3-cyclobutanedione, and combinations thereof.

In some embodiments, the second material is an inorganic substance that contains specific chemical elements having an incomplete electronic d-shell (i.e. atoms or ions of transition elements), and whose infrared absorption is a consequence of electronic transitions within the d-shell of the atom or ion. In some embodiments, the inorganic IR absorbing material comprises one or more transition metal elements in the form of an ion such as a palladium(II), a platinum(II), a titanium(III), a vanadium(IV), a chromium(V), an iron(II), a nickel(II), a cobalt(II) or a copper(II) ion (corresponding to the chemical formulas Ti³⁺, VO²⁺, Cr⁵⁺, Fe²⁺, Ni²⁺, Co²⁺, and Cu²⁺). In some embodiments, the second material is an inorganic IR absorbing material with near-infrared absorbing properties selected from the group consisting of zinc copper phosphate pigment ((Zn,Cu)₂P₂O₇), zinc iron phosphate pigment ((Zn,Fe)₃(PO₄)₂), magnesium copper silicate ((Mg,Cu)₂Si₂O₆ solid solutions), and combinations thereof. In some embodiments, the inorganic IR absorbing material is a zinc iron phosphate pigment. In some embodiments, the inorganic IR absorbing material may include palladate (e.g. barium tetrakis(cyano-C)palladate tetrahydrate, BaPd(CN)₄.4H₂O, [Pd(dimit)₂]²⁻, bis(1,3-dithiole-2-thione-4,5-dithiolate)palladate(II). In some embodiments, the inorganic IR absorbing material may include platinate, e.g. platinum-based polypyridyl complexes with dithiolate ligands, Pt(II)(diamine)(dithiolate) with 3,3′-, 4,4′-, 5,5′-bipyridyl substituents.

In some embodiments, the second material comprises iron oxide nanoparticle (also known to function as MM contrast agent, magnetic energy absorbing agent).

In some embodiments, the second material interacting with exogenous source comprises plasmonic absorbers. In some embodiments, the plasmonic absorbers comprise plasmonic nanomaterials of noble metal gold (Au), silver (Ag) and copper (Cu) nanoparticles doped with sulfur (S), selenium (Se) or tellurium (Te) having a plasmonic resonance at a NIR wavelength. In some embodiments, the plasmonic absorbers comprise gold nanostructures such as nanoporous gold thin films, or gold nanospheres, gold nanorods, gold nanoshells, gold nanocages, silver nanoparticles, Cu9S5 nanoparticle, and iron oxide nanoparticles. In some embodiments, the plasmonic absorbers comprise gold nanostructures.

Compared to non-metallic nanoparticles, plasmonic nanomaterials exhibit a unique photophysical phenomenon, called localized surface plasmon resonance (LSPR) because of the absorption of light at a resonant frequency. The plasmonic nanomaterials (e.g. noble metal nanostructures) show superior light absorption efficiency over conventional dye molecules. Upon exposure to with electromagnetic radiation, strong surface fields are induced due to the coherent excitation of the electrons in the metallic nanoparticles. By changing the structure (e.g. size) and shape, the LSPR frequency of the noble metal nanostructures can be tuned to shift the resulting plasmonic resonance wavelength in the NIR therapeutic window (750-1300 nm), where light penetration in the tissue is optimal. The endogenous absorption coefficient of the tissue in the NIR band is nearly two orders of magnitude lower than that in the visible band of EM spectrum. In some embodiments, the plasmonic absorbers may have a LSPR ranging from about 700 nm to about 900 nm. In some embodiments, the plasmonic absorbers may have a LSPR ranging from about 900 nm to about 1064 nm.

In some embodiments, the second material is admixed within the carrier to form a homogeneous dispersion or a solid solution. In some embodiments, the second material and the carrier may have oppositely charged functional group(s) (e.g. IR absorbing material is positively charged tetrakis aminium dye, and the carrier has negatively charged functional group such as carboxylate anion of polymethacrylate polymers) such that the second material attaches to the carrier via hydrogen bond or via ionic electrostatic interactions.

In some embodiments, the second material is selected from the group consisting of a tetrakis aminium dye, a cyanine dye, a squarylium dye, indocyanine green (ICG), new ICG (IR 820), squaraine dye, IR 780 dye, IR 193 dye, Epolight™ 1117 dye, Epolight™ 1175, iron oxide, gold nanoparticle, gold nanorod, gold nanofilm, gold nanocage, zinc iron phosphate pigment, and combinations thereof.

In some embodiments, the second material is a tetrakis aminium dye. In some embodiments, the tetrakis aminium dye is a narrow band absorber including commercially available dyes sold under the trademark names Epolight™ 1117 (peak absorption, 1071 nm), Epolight™ 1151 (peak absorption, 1070 nm), or Epolight™ 1178 (peak absorption, 1073 nm). In some embodiments, the tetrakis aminium dyes is a broadband absorber including commercially available dyes sold under the trademark names Epolight™ 1175 (peak absorption, 948 nm), Epolight™ 1125 (peak absorption, 950 nm), and Epolight™ 1130 (peak absorption, 960 nm). In some embodiments, the tetrakis aminium dye is Epolight™ 1178.

In some embodiments, the theragnostic particles comprise core particles of 100-200 nm in size formed from the carrier and the second material as described herein, and a thin layer of noble metal film (5-20 nm) as particle surface coatings, wherein the noble metal is selected from the group consisting of gold (Au) nanostructure, silver (Ag) nanoparticle, copper (Cu) nanoparticle having a plasmonic resonance at a NIR wavelength, and combinations thereof, wherein the theragnostic particle exhibits additive or synergistic PTT resulting from LSPR of film coated nanoparticle and the conventional PTT from the second material in the particle core. The LSPR wavelength is tunable by decreasing the shell thickness-to-core radius ratio, wherein LSPR wavelength shift is independent of shell size, core material, shell metal or surrounding medium.

In some embodiments, the theragnostic particles comprise core particles of 1000-2000 nm in size formed from the carrier and the second material as described herein, and a thin layer of noble metal film (50-200 nm) as particle surface coatings, wherein the noble metal is selected from the group consisting of gold (Au) nanostructure, silver (Ag) nanoparticle, copper (Cu) nanoparticle having a plasmonic resonance at a NIR wavelength, and combinations thereof, wherein the theragnostic particle exhibits additive or synergistic PTT resulting from LSPR of film coated nanoparticle and the conventional PTT from organic dye in the particle core. The LSPR wavelength is tunable by decreasing the shell thickness-to-core radius ratio, wherein LSPR wavelength shift is independent of shell size, core material, shell metal or surrounding medium.

In some embodiments, the theragnostic particles further comprise a shell to form core-shell particles, wherein the second material interacting with the exogenous source is plasmonic absorbers disposed in the shell, alternatively the plasmonic absorbers are embedded within, either ionically associated with, or covalently bonded to the shell. In some embodiments, the plasmonic absorbers are particles having a thin and porous gold wall with hollow interior, wherein the LSPR wavelength can be tuned by changing the wall thickness, pore size and porosity. In some embodiments, the plasmonic absorbers are core-shell particles having a gold nanoparticle core having the shape of sphere, shell, or rod, and a shell of hydrophilic polymer (e.g. chitosan, PEG) to enclose the gold nanoparticle core.

In some embodiments, the particle heater further comprises a shell to form a core-shell particle. In some embodiments, the core comprises a plasmonic absorber or iron oxide nanoparticles. In some embodiments, the shell comprises a plasmonic absorber or iron oxide. In some embodiments, the plasmonic absorber comprises plasmonic nanomaterials selected from the group consisting of noble metal including gold (Au) nanostructure, silver (Ag) nanoparticle, copper (Cu) nanoparticle having a plasmonic resonance at a NIR wavelength, and combinations thereof. In some embodiments, the shell comprises an agent selected from the group consisting of gold nanostructures, silver nanoparticles, iron oxide film, iron oxide nanoparticle, and combinations thereof.

Polydopamine is a biogenic material that is known to exhibit a photothermal response to infrared irradiation. Polydopamine has previously been applied as a surface coating onto silica particles. In some embodiments, the theragnostic particles comprise a coating made of polydopamine that is capable of converting exogenous energy into heat.

In some embodiments, the shell comprises an agent selected from the group consisting of inorganic polymers, organic polymers including polyureas or polyurethanes, silicates, mesoporous silica, organosilicate, organo-modified silicone polymers, cross-linked organic polymers, and combinations thereof. In some embodiments, the shell is formed of an agent selected from protein, polysaccharide, lipid, and combinations thereof.

In some embodiments, the shell comprises a crosslinked inorganic polymer. In some embodiments, the crosslinked inorganic polymer comprises organo-modified polysilicates. The shell may comprise inorganic polymers such as silicates, organosilicate, and organo-modified silicone polymer derived from condensation of organotrisilanol or halotrisilanol. The process to apply the crosslinked shell must be designed so as to maximize the stability of the particle heater components to the chemistry required in shell construction, at least until the growing shell protects the components encapsulated in the particle heater.

Therefore, in some embodiments, the present disclosure provides particle heaters having a core-shell structure to reduce particle porosity and to protect the material from the degradation by the body chemicals. Therefore, the stability of the material inside the particles are improved due to the reduced incursion of the body chemicals. In some embodiments, the shell comprises a crosslinked organo-silicate polymer derived from trialkoxysilane, or trihalorosilane, for example, to protect the IR absorbing material Epolight™ 1117 encapsulated in a NeoCryl® 805 particle when introduced into human skin, a sol-gel organo-modified silicate polymer shell derived from alkyltrimethoxysilane is formed on the surface of the polymeric particle to block the free exchange of nucleophiles and free radical species between the particles and the surrounding environment.

In some embodiments, the trialkoxysilane used for making the shell is selected from the group consisting of C2-C7 alkyl-trialkoxysilane, C2-C7 alkenyl-trialkoxysilane, C2-C7 alkynyl-trialkoxysilane, aryl-trialkoxysilane, and combinations thereof. In some embodiments, the trihalosilane used for making the shell is selected from the group consisting of trichlorosilane, tribromosilane, triiodosilane, and combinations thereof. In some embodiments, the crosslinked organo-silicate polymer is derived from vinyl-trimethoxysilane.

In some embodiments, the particle heaters further comprise microbial targeting groups as disclosed herein. In some embodiments, the microbial targeting group is cationically charged such that the particle heaters are localized to the membrane of the bacteria. In some embodiments, the cationically charged microbial targeting group is selected from the group consisting of vancomycin, cationic antimicrobial peptide, and combinations thereof.

In some embodiments, at least a portion of the exterior surface of the particle heater has a modification that is polar, non-polar, charged, ionic, basic, acidic, reactive, hydrophobic, or hydrophilic.

In some embodiments, the particle heater maintains integrity after interacting with the exogenous source. In some embodiments, the particle structure is altered after interacting with the exogenous source.

In some embodiments, the second material in the theragnostic particle is present in an amount ranging from about 5.0 wt. % to about 15.0 wt. % by the total weight of the theragnostic particle. In some embodiments, the second material of the theragnostic particle is present in an amount selected from the group consisting of about 5.0 wt. %, about 5.56 wt. %, about 10.4 wt. %, about 12.0 wt. %, about 12.1 wt. %, about 13.64 wt. %, about 14.0 wt. %, or about 15.0 wt. % by the total weight of the theragnostic particle. In some embodiments, the theragnostic particle comprises the second material in an amount of about 5.0 wt. %, about 5.25 wt. %, about 5.5 wt. %, about 5.75 wt. %, about 6.0 wt. %, 6.25 wt. %, about 6.5 wt. %, about 6.75 wt. %, about 7.0 wt. %, 7.25 wt. %, about 7.5 wt. %, about 7.75 wt. %, about 8.0 wt. %, about 8.25 wt. %, about 8.5 wt. %, about 8.75 wt. %, about 9.0 wt. %, about 9.25 wt. %, about 9.5 wt. %, about 9.75 wt. %, about 10.0 wt. %, about 10.25 wt. %, about 10.5 wt. %, about 10.75 wt. %, about 11.0 wt. %, about 11.25 wt. %, about 11.5 wt. %, about 11.75 wt. %, about 12.0 wt. %, about 12.25 wt. %, about 12.5 wt. %, about 12.75 wt. %, about 13.0 wt. %, about 13.25 wt. %, about 13.5 wt. %, about 13.75 wt. %, about 14.0 wt. %, about 14.25 wt. %, about 14.5 wt. %, about 14.75 wt. %, or about 15.0 wt. %.

The carrier may include a biocompatible material selected from the group consisting of a lipid, polymer-lipid conjugate, carbohydrate-lipid conjugate, peptide-lipid conjugate, protein-lipid conjugate, an inorganic polymer, polyester, a polyester, a polyurea, a polyanhydride, a polysaccharide, a polyphosphoester, a poly(ortho ester), a poly(amino acid), a protein, dendritic polylysine, and combinations thereof. In some embodiments, the carrier may be of any material described herein to be suitable as the carrier for the diagnostic particle.

In some embodiments, the theragnostic particle has a weight ratio of the carrier to the biosensor ranging from 1:1 to 7:1. In some embodiments, the theragnostic particle has a weight ratio of the carrier to the biosensor selected from the group consisting of 1.0:1, 1.1:1, 1.2:1, 1.3:1, 1.4:1, 1.5:1, 1.6:1, 1.7:1, 1.8:1, 1.9:1, 2.0:1, 2.1:1, 2.2:1, 2.3:1, 2.4:1, 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 41.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, and 7.0:1.

In some embodiments, the theragnostic particle has a weight ratio of the second material to the biosensor ranging from 7:1 to 1:7. In some embodiments, the theragnostic particle has a weight ratio of the carrier to the biosensor selected from the group consisting of 7.0:1, 6.9:1, 6.8:1, 6.7:1, 6.6:1, 6.5:1, 6.4:1, 6.3:1, 6.2:1, 6.1:1, 6.0:1, 5.9:1, 5.8:1, 5.7:1, 5.6:1, 5.5:1, 5.4:1, 5.3:1, 5.2:1, 5.1:1, 5.0:1, 4.9:1, 4.8:1, 4.7:1, 4.6:1, 4.5:1, 4.4:1, 4.3:1, 4.2:1, 4.1:1, 4.0:1, 3.9:1, 3.8:1, 3.7:1, 3.6:1, 3.5:1, 3.4:1, 3.3:1, 3.2:1, 3.1:1, 3.0:1, 2.9:1, 2.8:1, 2.7:1, 2.6:1, 2.5:1, 2.4:1, 2.3:1, 2.2:1, 2.1:1, 2.0:1, 1.9:1, 1.8:1, 1.7:1, 1.6:1, 1.5:1, 1.4:1, 1.3:1, 1.2:1, 1.1:1, 1.0:1, 1:1.1, 1:1.2, 1:1.3, 1:1.4, 1:1.5, 1:1.6, 1:1.7, 1:1.8, 1;1.9, 1:2.0, 1:2.1, 1:2.2, 1:2.3, 1:2.4, 1:2.5, 1:2.6, 1:2.7, 1:2.8, 1:2.9, 1:3.0, 1:3.1, 1:3.2, 1:3.3, 1:3.4, 1:3.5, 1:3.6, 1:3.7, 1:3.8, 1:3.9, 1:4.0, 1:4.1, 1:4.2, 1:4.3, 1:4.4, 1:4.5, 1:4.6, 1:4.7, 1:4.8, 1:4.9, 1:5.0, 1:5.1, 1:5.2, 1:5.3, 1:5.4, 1:5.5, 1:5.6, 1:5.7, 1:5.8, 1:5.9, 1:6.0, 1:6.1, 1:6.2, 1:6.3, 1:6.4, 1:6.5, 1:6.6, 1:6.7, 1:6.8, 1:6.9, and 1:7.0.

In some embodiments, the particle heater may have a spherical shape. In some embodiments, the particle heater may have cylindrical shape.

In some embodiments, the particle heater may have a wide variety of non-spherical shapes. The non-spherical shaped particle heater can be used to alter uptake by phagocytic cells and thereby clearance by the reticuloendothelial system. In some embodiments, the non-spherical particle heater may be in the shape of rectangular disks, high aspect ratio rectangular disks, rods, high aspect ratio rods, worms, oblate ellipses, prolate ellipses, elliptical disks, UFOs, circular disks, barrels, bullets, pills, pulleys, bi-convex lenses, ribbons, ravioli, flat pill, bicones, diamond disks, emarginated disks, elongated hexagonal disks, tacos, wrinkled prolate ellipsoids, wrinkled oblate ellipsoids, or porous elliptical disks. Additional shapes beyond those are also within the scope of the definition for “non-spherical” shapes.

In some embodiments, the particle heaters have a PdI from about 0.05 to about 0.15, about 0.06 to about 0.14, about 0.07 to about 0.13, about 0.08 to about 0.12, or about 0.09 to about 0.11. In some embodiments, the particle heaters have a PdI of about 0.05, about 0.06, about 0.07, about 0.08, about 0.09, about 0.10, about 0.11, about 0.12, about 0.13, about 0.14, or about 0.15

In some embodiments, the particle heater has a median size less than 1000 nm. In some embodiments, the particle heater has a median size ranging from about 1 nm to about 1000 nm. In some embodiments, the particle heater has a median size ranging from about 1 nm to about 500 nm. In some embodiments, the particle heater has a median size ranging from about 1 nm to about 250 nm. In some embodiments, the particle heater has a median size ranging from about 1 nm to about 150 nm. In some embodiments, the particle heater has a median size ranging from about 1 nm to about 100 nm. In some embodiments, the particle heater has a median size ranging from about 1 nm to about 50 nm. In some embodiments, the particle heater has a median size ranging from about 1 nm to about 25 nm. In some embodiments, the particle heater has a median size ranging from about 1 nm to about 10 nm. In some embodiments, the particle heater has a median size selected from the group consisting of about 1 nm, about 5 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, about 200 nm, about 205 nm, about 210 nm, about 215 nm, about 220 nm, about 225 nm, about 230 nm, about 235 nm, about 240 nm, about 245 nm, about 250 nm, about 255 nm, about 260 nm, about 265 nm, about 270 nm, about 275 nm, about 280 nm, about 285 nm, about 290 nm, about 295 nm, about 300 nm, about 310 nm, about 320 nm, about 330 nm, about 340 nm, about 350 nm, about 360 nm, about 370 nm, about 380 nm, about 390 nm, about 400 nm, about 410 nm, about 420 nm, about 430 nm, about 440 nm, about 450 nm, about 460 nm, about 470 nm, about 480 nm, about 490 nm, about 500 nm, about 525 nm, about 550 nm, about 575 nm, about 600 nm, about 625 nm, about 650 nm, about 675 nm, about 700 nm, about 725 nm, about 750 nm, about 775 nm, about 800 nm, about 825 nm, about 850 nm, about 875 nm, about 900 nm, about 925 nm, about 950 nm, about 975 nm, and about 1000 nm. In some embodiments, the particle heater has a median size of 500 nm. In some embodiments, the diagnostic particle has a median particle size of 250 nm. In some embodiments, the particle heater has a median size of 750 nm.

In some embodiments, the particle heaters are microparticles having a median particle size equal or greater than 1000 nm (1 micron). In some embodiments, the particle heaters have a median particle size selected from the group consisting of about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 105 μm, about 110 μm, about 115 μm, about 120 μm, about 125 μm, about 130 μm, about 140 μm, about 145 μm, about 150 μm, about 155 μm, about 160 μm, about 165 μm, about 170 μm, about 175 μm, about 180 μm, about 185 μm, about 190 μm, about 195 μm, about 200 μm, about 205 μm, about 210 μm, about 215 μm, about 220 μm, about 225 μm, about 230 μm, about 235 μm, about 240 μm, about 245 μm, about 250 μm, about 255 μm, about 260 μm, about 265 μm, about 270 μm, about 275 μm, about 280 μm, about 285 μm, about 290 μm, about 295 μm, about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, and about 500 μm. In some embodiments, the particle heater has a median particle size in a range from about 1 μm to about 500 μm. In some embodiments, the particle heater has a median particle size in a range from about 1 μm to about 250 μm. In some embodiments, the particle heater has a median particle size in a range from about 1 μm to about 100 μm. In some embodiments, the particle heater has a median particle size in the range from about 1 μm to about 50 μm. In some embodiments, the particle heater has a median particle size in a range from about 1 μm to about 25 μm. In some embodiments, the particle heater has a median particle size in a range from about 1 μm to about 10 μm. In some embodiments, the particle heater has a median particle size in a range from about 1 μm to about 6 μm. In some embodiments, the particle heater has a median particle size from about 1 μm, about 2 μm, about 3 μm, about 4 μm, about 5 μm, or about 6 μm. In some embodiments, the particle heater has a median particle size in the range from about 1 μm to about 4 μm.

In one embodiment, the zeta potential of the diagnostic particles is from about −60 mV to about 60 mV, from about −50 mV to about 50 mV, from about −30 mV to about 30 mV, from about −25 mV to about 25 mV, from about −20 mV to about 20 mV, from about −10 mV to about 10 mV, from about −10 mV to 5 mV, from about −5 mV to about 5 mV, or from about −2 mV to about 2 mV. In some embodiments, the zeta potential of the diagnostic particles is in a range selected from the group consisting of about −10 mV to about 10 mV, from about −5 mV to about 5 mV, and from about −2 mV to about 2 mV. In some embodiments, the diagnostic particle surface charge is neutral or near-neutral (i.e., zeta potential is from about −10 mV to about 10 mV). In some embodiments, the disclosure provides topical theragnostic formulations suitable for the treatment of microbial infections in a subject. In some embodiments, the topical theragnostic formulation may take the form selected from the group consisting of a cream, a lotion, an ointment, a hydrogel, a colloid, a gel, a foam, an oil, a milk, a suspension, a wipe, a sponge, a solution, an emulsion, a paste, a patch, a pladget, a swab, a dressing, a spray, a pad, and combinations thereof.

b. Optional Additive for Theragnostic Particle

In some embodiments the theragnostic particles may include inhibitors of enzymatic antioxidants as additive. The inhibitors of enzymatic antioxidant is selected from the group consisting of superoxide dismutase (SOD), catalase (CAT), glutathione peroxidase (GPx) and thioredoxin (Trx). These inhibitors include but are not limited by: LCS-1 (4,5-dichloro-2-m-tolylpyridazin-3(2H)-one, salicylic acid, 6-Amino-5-nitroso-3-methyluracil, ATN-224 (bis-choline tetrathiomolybdate); 2-ME (2-methoxyoestradiol); N—N′-diethyldithiocarbamate, 3-Amino-1,2,4-Triazole, ρ-Hydroxybenzoic acid, misonidazole, d-penicillamine hydrochloride, 1-penicillamine hydantoin, dl-Buthionine-[S, R]-sulfoximine (BSO), Au(I) thioglucose, and combinations thereof.

In some embodiments, the optional additive in the theragnostic particle is present in an amount ranging from about 0.1 wt. % to about 5.0 wt. % by the total weight of the theragnostic particle. In some embodiments, the optional additive in the theragnostic particle is present in an amount ranging from about 0.5 wt. % to about 1.5 wt. % by the total weight of the theragnostic particle. In some embodiments, the second material of the theragnostic particle is present in an amount selected from the group consisting of about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, and about 5.0 wt. %.

### Theragnostic Composition Containing Particle Heaters

In an embodiment, this disclosure provides a theragnostic composition comprising the biosensors, dendritic biosensors, or the diagnostic particles as disclosed herein and a particle heater composed of a carrier and a second material interacting with an exogenous energy source as described herein, wherein the theragnostic composition could be used to detect, localize, and destroy microbes using the exogenous source. If the diagnostic particle or the biosensor produces a detectable change of optical response that indicates the presence of drug-resistant microbes (e.g., a color change from colorless to a bright color (e.g., blue or violet), or from bright color to colorless), then an exogenous energy source is applied to the theragnostic composition to induce an energy-to-heat conversion, whereby localized hyperthermia is induced to quickly kill the microbes.

In some embodiments, the biosensor in the theragnostic composition is a compound selected from those disclosed in Table 1.

In some embodiments, this disclosure provides a theragnostic composition comprising (1) at least one biosensor disclosed herein, (2) a pharmaceutically acceptable medium, and (3) a particle heater having the carrier and the second material as described for the theragnostic particles; wherein the biosensor detects and locates drug resistant microbes to guide the application of the exogenous source, wherein the second material absorbs and converts the energy from the exogenous source into heat, wherein the heat induces localized hyperthermia that causes death of microbes, and further wherein the particle heater structure is constructed such that it passes the Extractable Cytotoxicity Test.

In some embodiments, the particle heaters are combined with at least two different biosensors that each is independently responsive to β-lactamase, erythromycin (macrolide) esterase, chloramphenicol (phenicol) hydrolase,

In some embodiments, this disclosure provides a theragnostic composition comprising (1) at least one dendritic biosensor disclosed herein, (2) a pharmaceutically acceptable medium, and (3) a particle heater having the carrier and the second material as described for the theragnostic particles; wherein the dendritic biosensor detects and locates drug resistant microbes to guide the application of the exogenous source, wherein the second material absorbs and converts the energy from the exogenous source into heat, wherein the heat induces localized hyperthermia that causes death of microbes, and further wherein the particle heater structure is constructed such that it passes the Extractable Cytotoxicity Test.

In some embodiments, the particle heaters are combined with at least two different dendritic biosensors that each is independently responsive to β-lactamase, erythromycin (macrolide) esterase, chloramphenicol (phenicol) hydrolase,

In some embodiments, this disclosure provides a theragnostic composition comprising (1) at least one diagnostic particle as disclosed herein, (2) a pharmaceutically acceptable medium, and (3) a particle heater having the carrier and the second material as described for the theragnostic particles; wherein the diagnostic particle detects and locates drug resistant microbes to guide the application of the exogenous source, wherein the second material absorbs and converts the energy from the exogenous source into heat, wherein the heat induces localized hyperthermia that causes death of microbes, and further wherein the particle heater structure is constructed such that it passes the Extractable Cytotoxicity Test.

In some embodiments, the particle heaters are combined with at least two different populations of diagnostic particles that each is independently responsive to β-lactamase, erythromycin (macrolide) esterase, chloramphenicol (phenicol) hydrolase,

In some embodiments, this disclosure provides a theragnostic composition comprising (1) at least one diagnostic particle or at least one biosensor as disclosed herein, (2) a pharmaceutically acceptable medium, (3) an antibiotic as disclosed herein, and (4) a particle heater having the carrier and the second material as described for the theragnostic particles; wherein the diagnostic particle detects and locates drug resistant microbes to guide the application of the exogenous source, wherein the second material absorbs and converts the energy from the exogenous source into heat, wherein the heat induces localized hyperthermia that causes death of microbes, and further wherein the particle structure is constructed such that it passes the Extractable Cytotoxicity Test.

In some embodiments, this disclosure provides a dry, removable, sterile multilayered wound dressing, wherein the dressing comprises a matrix holding the theragnostic particles as described herein, wherein the matrix is selected from film, hydrogel membrane, non-woven fabric, and woven fabric, wherein the matrix is made of a biocompatible material selected from the group consisting of gelatin sponge, calcium alginate, collagen, oxidized regenerated cellulose, and combinations thereof, wherein the theragnostic particles are dispersed within, embedded within or forms a coating on the matrix.

In some embodiments, the wound dressing is constructed as a band-aid form, where the theragnostic particles formulation containing layer is adhered to an adhesive backing layer. One or more additional layers of wound dressing materials including a layer containing super absorbents to wick the wound exudate.

In some embodiments, the theragnostic wound dressing is constructed as a patch for use in treatment of Herpes Labialis, said patch comprising a backing layer, and a layer of a skin-friendly adhesive, said adhesive comprises theragnostic particles as described above and hydrocolloid particles, and one or more additional layers contains super absorbents to wick the wound exudate.

### Method of Detecting Drug Resistant Microbes

The spread of β-lactamases between bacteria has increased the resistance of bacteria to β-lactam drugs. The administration of β-lactam drugs to patients with bacteria resistant to those drugs selects for those bacteria and leads to an increase in the transmission of β-lactamases. Thus, there is a need to rapidly detect bacteria expressing specific β-lactamases so that an appropriate therapeutic regimen is selected for a given patient and the likelihood of the spread of resistant bacteria is reduced.

In an embodiment, this disclosure provides a method for detecting the presence or absence of a drug resistant microbes in a sample comprising the steps of: (1) providing the biosensor as disclosed herein, (2) mixing the biosensor with the sample, (3) observing the absence or presence of an optical response in the sample, wherein the presence of the optical response indicates the presence of the drug resistant microbes, wherein the antimicrobial inactivating factors cause degradation of the first material to release the spectroscopic probe and result in a detectable optical response. In some embodiments, the method further comprises the step of quantifying the optical response and correlating it to the bacterial load at the infection site. In some embodiments, the optical response is the color change of the biosensor. In some embodiments, the optical response is a fluorescence signal. In some embodiments, the method further comprises the step of quantifying the color change using an imaging colorimeter and correlating it to the bacterial load at the infection site. In some embodiments, the method further comprises the step of quantifying the fluorescence using an fluorimeter and correlating it to the bacterial load at the infection site.

In some embodiments, the optical response is a color change within the visible region of the electromagnetic spectrum. In some embodiments, the optical response is fluorescence.

In some embodiments, the method further comprising a step of quantifying the optical response using imaging spectroscopy for determining the bacterial burden to inform antibiotic selection and dosage thereof.

In some embodiments, this disclosure provides methods of detecting drug resistant microbes secreting antimicrobial inactivating factors by using three different population of biosensors: a first population of biosensors having a first material responsive to β-lactamase, a second population of biosensors having a first material responsive to erythromycin (macrolide) esterase, and a third population of biosensors having a first material responsive to amphenicol hydrolase.

In some embodiments, this disclosure provides methods of detecting drug resistant microbes secreting β-lactamases by using two or more biosensors for example, a first population of biosensors have a first material only being responsive to ESBL and OSBL but not a class A serine carbapenemase, a second population of biosensors have a first material only being responsive to penicillinase.

In some embodiments, the detection method for drug resistant microbes uses three different biosensors responsive to three different antibiotic inactivating factors, wherein the first population of biosensors have a first material only being responsive to β-lactamase, the second population of biosensors have a first material only being responsive to erythromycin esterase, and the third population of biosensors have a first material only being responsive to chloramphenicol (phenicol) hydrolase.

In some embodiments, the detection method for drug resistant microbes uses three different biosensors responsive to three different sub-family of β-lactamases, wherein the first population of biosensors have a first material only being responsive to AmpC β-lactamases, the second population of biosensors have a first material only being responsive to Extended-spectrum β-lactamases (ESBLs), and the third population of biosensors have a first material only being responsive to carbapenemases.

In some embodiments, the detection method for drug resistant microbes uses three different biosensors responsive to two different carbapenemases, wherein the first population of biosensors have a first material only being responsive to metallo-β-lactamases; the second population of biosensors have a first material only being responsive to extended spectrum β-lactamases (ESBL).

In some embodiments, the detection method for drug resistant microbes uses three different biosensors responsive to three different β-lactamases, wherein the first population of biosensors have a first material only being responsive to penicillinases, the second population of biosensors have a first material only being responsive to cephalosporinases, and the third population of biosensors have a first material only being responsive to carbenicillinases.

In some embodiments, this disclosure provides methods of detecting drug resistant microbes that secrete β-lactamases by using two or more biosensors having a bimodal β-lactamase sensing component composed of a conjugate of a β-lactam antibiotic fragment with a β-lactamase inhibitor fragment. In some embodiments, the β-lactamase inhibitor fragment is derived from an AmpC inhibitor. In some embodiments, the β-lactamase inhibitor fragment is derived from a serine β-lactamase inhibitor in an amount sufficient to inhibit ESBL and an OSBL but not a class A serine carbapenemase. In some embodiments, the β-lactamase inhibitor fragment is derived from an AmpC inhibitor and a serine β-lactamase inhibitor in an amount sufficient to inhibit ESBL and an OSBL but not a class A serine carbapenemase. In some embodiments, the β-lactamase inhibitor fragment is derived from an ESBL inhibitor.

In some embodiments, this disclosure provides a method for detecting the presence or absence of a drug resistant microbes in a sample comprising the steps of: (1) providing the dendrimer biosensors diagnostic particles as disclosed herein, (2) incubating the dendrimer biosensorsdiagnostic particles with the sample, (3) observing the absence or presence of an optical response in the sample, wherein the presence of the optical response indicates the presence of the drug resistant microbes, wherein the antimicrobial inactivating factors cause degradation of the first material to release the spectroscopic probe and result in a detectable optical response.

In some embodiments, the detection method for drug resistant microbes uses three different dendrimer biosensors responsive to three different antibiotic inactivating factors, wherein the first population of dendrimer biosensors have a first material only responsive to β-lactamase, the second population of dendrimer biosensors have a first material only responsive to erythromycin esterase, and the third population of dendrimer biosensors have a first material only responsive to chloramphenicol (phenicol) hydrolase.

In some embodiments, the detection method for drug resistant microbes uses three different dendrimer biosensors responsive to three different sub-family of β-lactamases, wherein the first population of dendrimer biosensors have a first material only responsive to AmpC β-lactamases, the second population of dendrimer biosensors have a first material only responsive to Extended-spectrum β-lactamases (ESBLs), and the third population of dendrimer biosensors have a first material only responsive to carbapenemases.

In some embodiments, the detection method for drug resistant microbes uses three different dendrimer biosensors responsive to two different carbapenemases, wherein the first population of dendrimer biosensors have a first material only responsive to metallo-β-lactamases; the second population of dendrimer biosensors have a first material only being responsive to extended spectrum β-lactamases (ESBL).

In some embodiments, the detection method for drug resistant microbes uses three different dendrimer biosensors responsive to three different β-lactamases, wherein the first population of dendrimer biosensors have a first material only being responsive to penicillinases, the second population of dendrimer biosensors have a first material only being responsive to cephalosporinases, and the third population of dendrimer biosensors have a first material only being responsive to carbenicillinases.

In some embodiments, this disclosure provides a method for detecting the presence or absence of a drug resistant microbes in a sample comprising the steps of: (1) providing the diagnostic particles as disclosed herein, (2) incubating the diagnostic particles with the sample, (3) observing the absence or presence of an optical response in the sample, wherein the presence of the optical response indicates the presence of the drug resistant microbes, wherein the antimicrobial inactivating factors cause degradation of the first material to release the spectroscopic probe and result in a detectable optical response.

In some embodiments, the detection method for drug resistant microbes uses three different diagnostic particles responsive to three different antibiotic inactivating factors, wherein the first population of diagnostic particles have a first material only responsive to β-lactamase, the second population of diagnostic particles have a first material only responsive to erythromycin esterase, and the third population of diagnostic particles have a first material only responsive to chloramphenicol (phenicol) hydrolase.

In some embodiments, the detection method for drug resistant microbes uses three different diagnostic particles responsive to three different sub-family of β-lactamases, wherein the first population of diagnostic particles have a first material only responsive to AmpC β-lactamases, the second population of diagnostic particles have a first material only responsive to Extended-spectrum β-lactamases (ESBLs), and the third population of diagnostic particles have a first material only responsive to carbapenemases.

In some embodiments, the detection method for drug resistant microbes uses three different diagnostic particles responsive to two different carbapenemases, wherein the first population of diagnostic particles have a first material only responsive to metallo-β-lactamases; the second population of diagnostic particles have a first material only being responsive to extended spectrum β-lactamases (ESBL).

In some embodiments, the detection method for drug resistant microbes uses three different diagnostic particles responsive to three different β-lactamases, wherein the first population of diagnostic particles have a first material only being responsive to penicillinases, the second population of diagnostic particles have a first material only being responsive to cephalosporinases, and the third population of diagnostic particles have a first material only being responsive to carbenicillinases.

In some embodiments, the diagnostic particle containing a first material having the combination of a β-lactam fragment and a β-lactamase inhibitor fragment is useful for rapid antibiotic susceptibility testing. In some embodiments, two or more different populations of the diagnostic particles may be used for rapid antibiotic susceptibility profiling.

In some embodiments, the methods, compositions, kits and systems described herein can allow determination of antibiotic susceptibility of a microbe based on a small number of microbes, e.g., as few as 5-10 microbes bound to a biosensor as disclosed herein.

In some embodiments, this disclosure provides a method for determining antibiotic susceptibility of a microbe comprising: (i) obtaining a sample suspected of comprising a microbe, wherein the microbe has been extracted or concentrated from a test sample using a targeted diagnostic particle having a microbe-targeting group bound to the particle surface as disclosed herein; (ii) incubating the targeted diagnostic particle in the presence of at least one antibiotic agent for a pre-determined period of time; and (iii) detecting the growth or functional response of the microbe to the antibiotic agent, wherein reduced growth or function in the presence of the antibiotic agent relative to a reference or control sample indicates that the microbe is susceptible to the antibiotic agent.

In some embodiments, this disclosure provides a method for determining antibiotic susceptibility of a microbe by mixing three biosensors (BS), the first biosensor responsive to microbes secreting β-lactamases (BS1), a second biosensor responsive to microbes secreting macrolide esterases (BS2) and the third biosensor responsive to microbes secreting amphenicol hydrolases (BS3). The optical response obtained from using this mixture can be used to guide subsequent antibiotic therapy. For e.g. if the microbes present at the infection site only cause an optical response for BS1, this indicates the presence of only β-lactamase producing microbes in the subject at the infection site, so either a macrolide-containing antibiotic (e.g. erythromycin) or an amphenicol-containing antibiotic (chloramphenicol) or a combination of the two may be administered to the subject to successfully treat the microbial infection. If the microbes present at the infection site produce an optical response to BS1 and BS2, this indicates the presence of both β-lactamase and macrolide esterase producing microbes in the subject, so an amphenicol-containing antibiotic (chloramphenicol) may be administered to the subject to successfully treat the microbial infection. If the microbes present at the infection site produce an optical response to all three biosensors (i.e. BS1, BS2 and BS3), this indicates the presence of β-lactamase, macrolide esterase and amphenicol hydrolase producing microbes in the subject suggesting the need for alternative treatments. This may include any antibiotic not containing a β-lactamase, macrolide, or an amphenicol. Alternatively, theragnostic particles can be used to kill these multidrug resistant microbes using hyperthermia following interaction with the exogenous source.

In some embodiments, this disclosure provides a method for determining antibiotic susceptibility of a microbe by mixing three populations of diagnostic particles (DP), the first diagnostic particle containing a biosensor responsive to microbes secreting β-lactamases (DP1), a second diagnostic particle containing a biosensor responsive to microbes secreting macrolide esterases (DP2) and the third diagnostic particle containing a biosensor responsive to microbes secreting amphenicol hydrolases (DP3). The optical response obtained from using this particle mixture can be used to guide subsequent antibiotic therapy. For e.g. if the microbes present at the infection site only cause an optical response for DP1, this indicates the presence of only β-lactamase producing microbes in the subject at the infection site, so either a macrolide-containing antibiotic (e.g. erythromycin) or an amphenicol-containing antibiotic (chloramphenicol) or a combination of the two may be administered to the subject to successfully treat the microbial infection. If the microbes present at the infection site produce an optical response to DP1 and DP2, this indicates the presence of both β-lactamase and macrolide esterase producing microbes in the subject, so an amphenicol-containing antibiotic (chloramphenicol) may be administered to the subject to successfully treat the microbial infection. If the microbes present at the infection site produce an optical response to all three diagnostic particles (i.e. DP1, DP2 and DP3), this indicates the presence of β-lactamase, macrolide esterase and amphenicol hydrolase producing microbes in the subject suggesting the need for alternative treatments. This may include any antibiotic not containing a β-lactamase, macrolide, or an amphenicol. Alternatively theragnostic particles can be used to kill these multidrug resistant microbes using hyperthermia following interaction with the exogenous source.

In some embodiments, this disclosure provides a method for diagnosing and killing of drug resistant microbes at a site. The method may include administering an effective amount of a theragnostic particles disclosed herein to the site, contacting the theragnostic particles with a milieu near the site, waiting for a period of time to observe the presence or absence of optical response, and when an optical response is observed, indicating the presence of the drug resistant microbes and employing an exogenous source at the site. The theragnostic particles absorb the energy from the exogenous source and converts the absorbed energy into heat. The heat travels outside the theragnostic particle to induce localized hyperthermia at a temperature ranging from about 38° C. to about 52° C. in an area surrounding the theragnostic particle. The localized hyperthermia lasts for a sufficient period of time to cause the death of the drug resistant microbes.

In some embodiments, the site corresponds to an infection site associated with a subject.

In some embodiments, this disclosure provides a method for determining antibiotic susceptibility of a microbe by mixing three populations of theragnostic particles (TP), the first theragnostic particle containing a biosensor responsive to microbes secreting β-lactamases (TP1), a second theragnostic particle containing a biosensor responsive to microbes secreting macrolide esterases (TP2) and the third diagnostic particle containing a biosensor responsive to microbes secreting amphenicol hydrolases (TP3). The optical response obtained from using this particle mixture can be used to guide subsequent antibiotic therapy. For e.g. if the microbes present at the infection site only cause an optical response for TP1, this indicates the presence of only β-lactamase producing microbes in the subject at the infection site, so either a macrolide-containing antibiotic (e.g. erythromycin) or an amphenicol-containing antibiotic (chloramphenicol) or theragnostic particle mediated hyperthermia or a combination of the three may be used to successfully treat the microbial infection. If the microbes present at the infection site produce an optical response to TP1 and TP2, this indicates the presence of both β-lactamase and macrolide esterase producing microbes in the subject, so an amphenicol-containing antibiotic (chloramphenicol) or theragnostic particle mediated hyperthermia may be used to successfully treat the microbial infection. If the microbes present at the infection site produce an optical response to all three theragnostic particles (i.e. TP1, TP2 and TP3), this indicates the presence of β-lactamase, macrolide esterase and amphenicol hydrolase producing microbes in the subject suggesting the need for alternative treatments. This may include any antibiotic not containing a β-lactamase, macrolide, or an amphenicol. Alternatively theragnostic particles can be used to kill these multidrug resistant microbes using hyperthermia following interaction with the exogenous source.

In some embodiments, the targeted diagnostic particle comprises a biosensor having a bimodal first material with a combination of a β-lactam antibiotic fragment and a β-lactamase inhibitor.

In some embodiments, the microbial-targeting group is selected from the group consisting of vancomycin, a microbial-binding portion of C-type lectins, ColCol-like lectins, ficolins, receptor-based lectins, lectins from the shrimp marsupenaeus japonicas, non-C-type lectins, a lipopolysaccharide (LPS)-binding proteins, endotoxin-binding proteins, mannan-binding lectin (MBL), surfactant protein A, surfactant protein D, collectin 11, L-ficolin, ficolin A, DC-SIGN, DC-SIGNR, SIGNR1, macrophage mannose receptor 1, dectin-1, dectin-2, lectin A, lectin B, lectin C, wheat germ agglutinin, CD 14, MD2, lipopolysaccharide-binding protein (LBP), limulus anti-LPS factor (LAL-F), mammalian peptidoglycan recognition protein-1 (PGRP-1), PGRP-2, PGRP-3, PGRP-4, and combinations thereof. In some embodiments, the microbial-targeting group is a cationic antimicrobial peptide. In some embodiments, the microbial-targeting group is vancomycin. In some embodiments, the microbe-targeting group is a LPS binding protein.

In some embodiments, two or more different population of diagnostic particles responsive to β-lactamase, macrolide esterase, and amphenicol hydrolase are applied in the method for determining antibiotic susceptibility of a microbe.

In some embodiments, this disclosure provide a kit for determining antibiotic susceptibility of a microbe comprising at least three different targeted diagnostic particles responsive to β-lactamase, macrolide esterase, and amphenicol hydrolase. This kit will include a diagram showing a also includes an instruction sheet illustrating the correlation between the optical response of the diagnostic particles and the drug resistant microbes detected. The kit also include an instruction sheet for interpreting the optical response.

In some embodiments, the sample includes a biological fluid, a tissue sample, a tissue section, a cell sample, non-biological fluid, a surface or a substrate. In some embodiments, the sample is a wound site in a subject. In some embodiments, the subject is a human or an animal. In some embodiments, the sample is a wound care device such as wound dressing. In some embodiments, the sample is a human sample. In some embodiments, the sample is an animal sample.

In some embodiments, the method uses a bimodal biosensor at a concentration of about 20 μM to 200 μM.

In some embodiments, the method is conducted at a pH value of about 5 to about 8, about 5.5 to about 7.5, or 6 to about 7.

In some embodiments, the method is conducted at a temperature of about 20° C. to about 42° C., about 25° C. to about 40° C., or about 37° C.

In some embodiments, the detection step of the methods may be performed for the minimum amount of time that is needed for the minimum amount of biosensor to be utilized by a bacterial sample to produce a detectable optical response (e.g., visual observation and/or spectrophotometry). In some embodiments, the detectable optical response is measured using an instrument such as, for example, a multi-well plate reader.

In some embodiments, the detection step of the methods is performed for a time period selected from the group consisting of about 2 minutes to about 60 minutes, about 2 minutes to about 45 minutes, about 2 minutes to about 30 minutes, about 2 minutes to about 15 minutes, and about 2 minutes to about 10 minutes after the contacting step. In some embodiments, the detection step of the methods is performed for a time period selected from the group consisting of about 15 minutes to about 5 hours, about 30 minutes to about 5 hours, about 30 minutes to about 4 hours, about 30 minutes to about 5 hours, about 1 hour to about 2 hours, about 1 hour to about 3 hours, about 1 hour to about 4 hours, about 1 hour to about 5 hours, about 2 hours to about 4 hours, about 2 hours to about 5 hours, and about 2 hours to about 6 hours after the contacting step. In some embodiments, the detection step is performed after each bacterial sample from the same source is contacted with each composition for the same amount of time.

In some embodiments, the bacterial concentration of bacterial in the sample is of at least 10² colony-forming units (CFU/mL). In some embodiments, the bacterial concentration detectable by the biosensor is selected from the group consisting of at least 10³ CFU/mL, at least 10⁴ CFU/mL, at least 10⁵ CFU/mL, at least 10⁶ CFU/mL, and at least 10⁷ CFU/mL of bacteria. In some embodiments, the bacterial concentration detectable by the biosensor is selected from the group consisting of about 10³ CFU/mL to about 10¹² CFU/mL, about 10⁵ CFU/mL to about 10¹² CFU/mL, about 10⁶ CFU/mL to about 10¹² CFU/mL, and about 10⁷ CFU/mL to about 10¹² CFU/mL. In some embodiments, the bacterial concentration detectable by the biosensor is of about 10⁷ CFU/mL to about 10¹⁰ CFU/mL of bacteria. In some embodiments, the bacterial concentration detectable by the biosensor is of 10³ CFU/mL or less. In some embodiments, the bacterial concentration detectable by the biosensor is of 10⁴ CFU/mL or less. In some embodiments, the bacterial concentration detectable by the biosensor is of 10⁶ CFU/mL or less.

In some embodiments, in addition to a bacterial sample to be tested for the presence of particular β-lactamases, a bacterial sample that is known to express one or more particular β-lactamases (i.e., a positive control) is included. In some embodiments, in addition to a bacterial sample to be tested for the presence of particular β-lactamases, a bacterial sample that is known to not express one or more particular β-lactamases (i.e., a negative control) is included. In some embodiments, in addition to a bacterial sample to be tested for the presence of particular β-lactamases, a bacterial sample that is known to express one or more particular β-lactamases (i.e., a positive control) and a bacterial sample that is known to not express one or more particular β-lactamases (i.e., a negative control) are included. For example, a positive control for an AmpC β-lactamase can be ATCC No. 700603 and a negative control can be ATCC No. 25922. Positive and negative controls for the assays may be identified using molecular and/or biochemical methods, such as sequencing, to determine the expression of a particular β-lactamase by a particular bacterial strain.

In some embodiments, this disclosure provides rapid, single and multiple drug resistant pathogen detection via a direct swab from the skin, saliva, mucous membrane, wounds, urine and faeces, after being exposed to the biosensors capable of producing optical response. An optical response after a set period indicates a positive result for the pathogen and would enable the patient to be rapidly isolated and treated using effective treatment protocols. This form of direct sampling and identification would facilitate a rapid methodology that can be used at the bedside or as a pre-admission screen. This represents a significant advancement in the early diagnosis/screening for drug resistant microbes or antimicrobial susceptibility profile screen. The sooner a drug-resistant infection is identified, the sooner an effective method of treatment or containment can be put in place.

In some embodiments, the bacteria are Gram-negative or Gram-positive bacteria. In some embodiments, the bacteria are Gram-negative bacteria. In some embodiments, the bacteria are Gram-positive bacteria. In some embodiments, the Gram-negative bacteria is selected from the group consisting of enterobacterial cells (Enterobacteriaceae), non-fermenting Gram-negative bacteria cells (such as for instance Acinetobacter spp and Pseudomonas spp), and combinations thereof. In some embodiments, the bacteria is selected from the group consisting of Acinetobacter spp including baumannii, pittii, hemolitycus and junii, Aeromonas caviae, Citrobacter amalonaticus, Citrobacter braakii, Citrobacter freundii, Citrobacter youngae, Enterobacter aerogenes, Enterobacter asburiae, Enterobacter cloacae, Escherichia coli, Klebsiella oxytoca, Klebsiella pneumoniae, Morganella morganii, Proteus mirabilis, Proteus rettgeri, Proteus vulgaris, Providencia stuartii, Providencia vermicola, Pseudomonas spp. including aeruginosa and putida, Salmonella enterica, Serratia marcescens, Shigella flexneri, and combinations thereof.

In a particular embodiment, the method of the present invention is used for detecting carbapenemase-producing bacteria including Enterobacteriaceae and Gram-negative non-fermenting bacteria.

In an embodiment, this disclosure provides a kit for detecting the presence of drug resistant bacteria, comprising: the biosensor composition described herein; and an instruction sheet providing instructions to a human subject, wherein the instructions comprise (1) collect a sample; (ii) contact the biosensor composition with the sample; and (iii) observe the presence or absence of the optical response.

In some embodiments, this disclosure provides a kit for detecting and killing drug-resistant bacteria, comprising: the theragnostic composition described herein; and an instruction sheet providing instructions to a human subject, wherein the instructions comprise (1) identify the site; (ii) contact the theragnostic composition with the site; (iii) observe the presence or absence of an optical response; and (iv) upon observing the optical response, expose the sample to an exogenous source.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### General Procedures

The compositions of this invention may be made by various methods known in the art. Such methods include those of the following examples, as well as the methods specifically exemplified below. Modifications of such methods that involve techniques commonly practiced in the art of sensors and particle technology may be used.

### Example 1 (i). Synthetic Scheme for the Preparation of the Biosensors

As used herein the symbols and conventions used in these processes, schemes and examples are consistent with those used in the contemporary scientific literature, for example, the Journal of the American Chemical Society or the Journal of Biological Chemistry. Specifically, the following abbreviations may be used in the examples and throughout the specification:

The following examples describe the invention in further detail, with reference to specific embodiments. These are representative embodiments of the invention which are provided for illustrative purposes only, and which should not be regarded as limiting the invention in any way.

Compounds of formula (1) or (2) where all variables are as defined herein can be prepared according to Scheme 1:

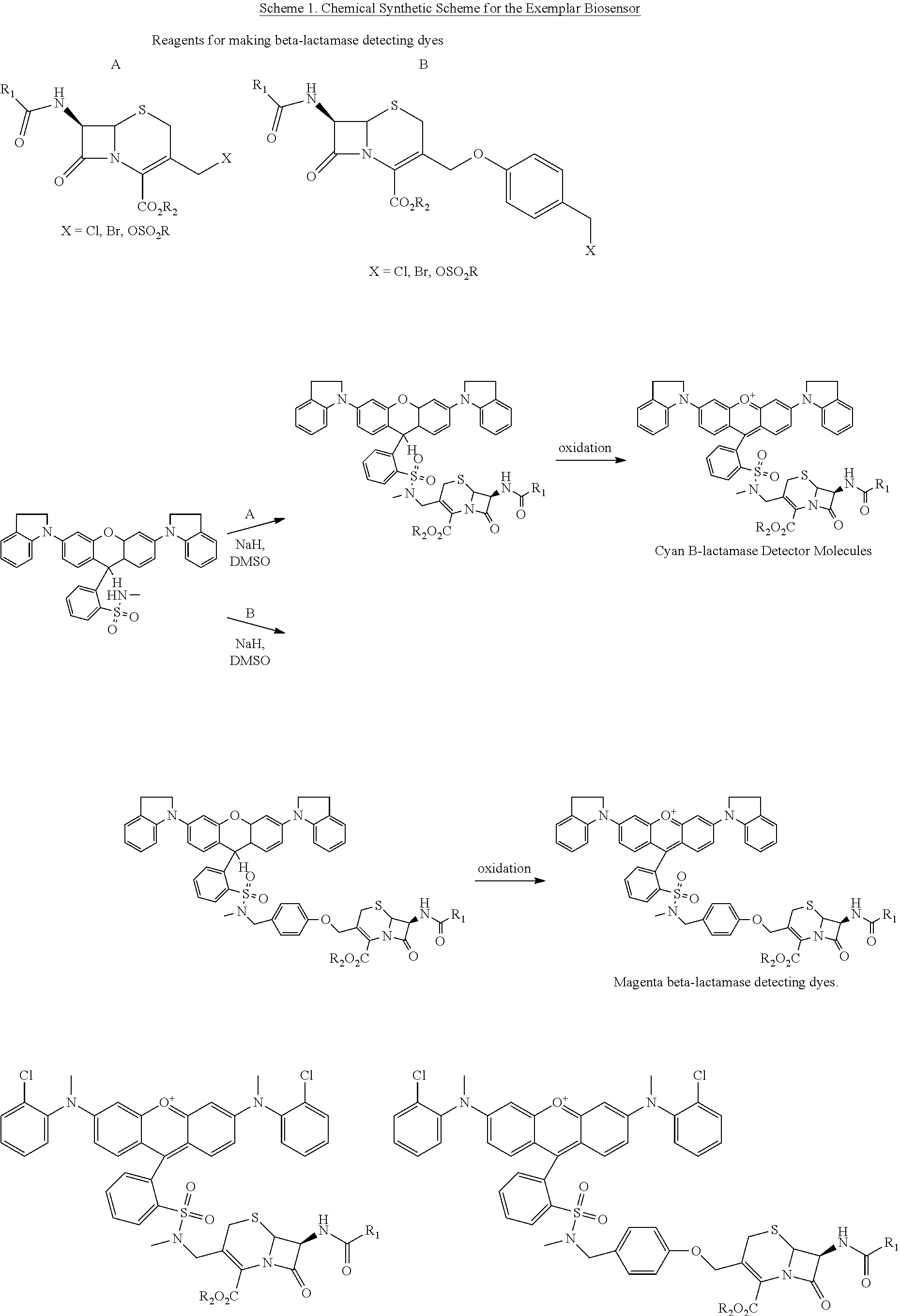

### Example 1 (ii) Synthesis of Dithioflurescein-Cephalosporin Conjugate (Formula (12))

The Formula (12) conjugate was prepared by convergent synthesis in four steps from Fluorescein and cephalosporin.

Fluorescein (13.29 g, 40.00 mmol) was suspended in DMF (40 mL) and POCl3 (18.64 mL, 200.0 mmol) was added to it while being stirred at room temperature under N₂. The solution was heated to 115° C. and stirred at the same temperature for 1 h. The mixture partially turned into a solid. The reaction mixture was then allowed to cool to room temperature and water (400 mL) was added. The contents were mixed well and centrifuged (5000 rpm, 10 min). The supernatant was discarded and more water (300 mL) was added to the solid, mixed well and centrifuged again (5000 rpm, 10 min). This step was repeated two more times. The final solid was then air dried for about 2 h and then dried in vacuum oven until all the water has been removed (about 2 days) to obtain 3′,6′-dichlorofluorecein as a light-brown (or off-white) solid (13 g, 88%).

To a solution of 3′,6′-dichlorofluorecein (2.77 g 7.50 mmol) in ethanol (50 mL), was added NaSH.xH₂O (6.0 g, excess) while stirring at room temperature under N₂. The mixture was then refluxed for 90 min. The mixture was then allowed to reach to room temperature and the solvent was evaporated. To the solid residue, 0.1 N HCl, containing 5% Na₂S₂O₅ was added while vigorous stirring. The mixture was then filtered and the solid was washed with 0.1 HCl. The solid was then dried in a vacuum oven for 2 days resulting the product as a beige color solid (2.9 g, quant.)

Cephalosporin (5.84 g, 12.0 mmol) was suspended in dry acetone (200 mL) under N₂. Sodium iodide (12.6 g, 84.0 mmol) was added to it and stirred at room temperature for 3 h. The solvent was evaporated under vacuum. The solid was dissolved in EA (200 mL) and washed with water (100 mL×3) and brine (50 mL×1). The organic layer was dried over sodium sulfate and the solvent was evaporated to yield iodocephalosporin as a yellow-brown foamy solid (6.2 g, 89%)

To 3′,6′-dithiofluorescein (728 mg, 2.00 mmol) and iodocephalosporin (2.54 g, 4.40 mmol) in acetonitrile (50 mL), was added K₂CO₃ (608 mg, 4.40 mmol) and stirred at room temperature for 5 h. The solvent was evaporated and DCM (30 mL) was added to the solid. The mixture was filtered and the solvent of the filtrate was evaporated. Very small amount of CP-I was observed on TLC. The product Formula (12) conjugate was used for color change studies without further purification and without deprotection.

Reaction of Formula (12) Conjugate with β-Lactamase

In four different vials, compound 4 (1.0 mg in each vial) was dissolved in DMSO (0.3 mL). To one vial, 25 μL of β-lactamase blend was added. To another vial 25 μL PBS was added as the negative control. To the other two vials 25 μL of papain and chymotrypsin were added separately. The vial that was treated with β-lactamase started changing color from light yellow to brownish-red within a minute and showed a very strong color change in less than 5 minutes. The vial that was treated with papain slightly changed color to light brown. Chymotrypsin resulted in almost no color change even after 30 minutes and the PBS control did not change color at all.

The color dyes disclosed in this application have been described in the U.S. Pat. Nos. 6,951,952, and 7,279,264, herein each is incorporated by reference by its entity. The leuco methylene blue dye is commercially available and has CAS No. 129094-52-6.

### Example 2. Particle Fabrication

Reagents source: Chemical reagents sodium dodecyl sulfate (SDS), aqueous polyvinyl alcohol (PVA), NeoCryl® B-805 polymer (MMA/BMA copolymer, weight average molecular weight=85,000 Da, glass transition temperature Tg=99° C.) was purchased from DSM. Epolight™ 1117 (tetrakis aminium, absorbing at 800 nm-1071 nm, melting point: 185-188° C., soluble in acetone, methylethylketone and cyclohexanone) was purchased from Epolin Inc. Antioxidant Cyanox® 1790 (1,3,5-tris(4-tert-butyl-3-hydroxy-2,6-dimethyl benzyl)-1,3,5-triazine-2,4,6-(1H,3H,5H)-trione, CAS NUMBER 040601-76-1) was purchased from Cytec Industries Inc.

### Example 2 (i) Synthesis and Characterization of Tetrakis Aminium Dye/B805 Particles (Uncoated Particles Synthesized Through Emulsion Method)

Abbreviations: n-BMA: n-butyl methacrylate; MMA: methyl methacrylate

The preparation of the aqueous phase: under the stirring with an IKA Ultra-Turrax® T 25 homogenizer at 8000 RPM, 1.2 g of sodium dodecyl sulfate (SDS) was added into 190 g of 4.9% aqueous polyvinyl alcohol (PVA) solution placed in a round bottom flask. An aqueous solution of SDS containing 4.9% PVA was formed after the dissolution of SDS (the aqueous phase).

The preparation of the organic phase: to 88 g of dichloromethane was added 8.0 g of DSM NeoCryl® B-805 polymer (MMA/BMA copolymer), 1.82 g of Epolight™ 1117 dye, and 0.65 g of Cyanox® 1790 in 88 g to allow the formation of a clear solution of B805 polymer and dyes (the polymer: dye weight ratio=4.4:1).

The organic phase (polymer and dyes dissolved in dichloromethane) was injected directly into the aqueous phase (PVA solution with SDS surfactant) at the tip of the Turrax's roto-stator (i.e. directly into the flow being sheared by the roto-stator). The shear mixing at 8000 RPM was continued for 30 minutes. The resulting mixture was decanted into an open-mouth container and stirred magnetically for 16 hours. A solid suspension of particles containing IR dye was obtained.

The solid suspension was centrifuged at 5000 RPM for 30 minutes and the particles were collected. The collected particles were washed with distilled water by resuspending the particles into distilled water and centrifuging as before to collect the particles. This washing process was repeated three times to remove residual PVA. The resulting MMA/BMA copolymer particles containing IR dye were air-dried.

### Example 2 (ii) Synthesis of 25% VTMS Coated Tetrakis Aminium Dye/B805 Particles

In a first vessel, 1.52 g (0.01 mmol) of vinyltrimethoxysilane (CH₂=CHSi(OMe)₃, VTMS, MW=148 Da) was mixed with 4.58 g of dilute aqueous hydrochloric acid at a pH of 3.5 under magnetic stirring (24.9 wt. % solution of CH₂=CHSi(OMe)₃ in diluted HCl). The resulting mixture was stirred for 2 hours to allow complete hydrolysis of VTMS to give vinylsilanetriol (CH₂=CHSi(OH)₃, MW=106 Da).

In a second vessel, under magnetic stirring, 3.0 g of pre-made uncoated IR dye particles of Example 1 (i) were dispersed in 57 grams of water to provide a 5.0 wt. % dye particle dispersion. The pH value of the resulting IR dye particle aqueous dispersion was adjusted to 10.0 with the addition of dilute aqueous ammonium hydroxide. To this particle dispersion at pH 10, an aliquot of 3.99 g of the hydrolyzed 25 wt. % VTMS solution was added at a rate of 2 drops per second to the particle suspension. The pH value of the resulting suspension was monitored after the hydrolyzed 25% VTMS solution addition and adjusted with ammonium hydroxide solution to maintain a pH of 10 for 60 minutes. After 60 minutes, the suspension was neutralized with glacial acetic acid to lower the pH from 10 to 4.6-5.7. The weight ratio of VTMS to the uncoated particle was 0.33:1.

The resulting particle suspension was centrifuged for 30 minutes at 5000 RPM to collect the vinylsilicate-coated dye particles. The particles collected after the centrifugation were redispersed in distilled water and subjected to centrifugation to collect the particles. The washing procedure was repeated 3 times to remove any unreacted chemical reagents. The resulting vinylsilicate-coated particles were suspended in distilled water.

Multiple commercially available infrared dyes were screened to find a preferred composition to provide localized heat delivery to a tissue site with sufficient temperature rise to accelerate a reaction outside of the particle. The infrared dyes screened include Lumogen IR 1050, Epolight™ 1117, Epolight™ 1125, and Epolight™ 1178.

In the emulsion method of encapsulation, a surfactant is necessary to help keep the emulsion stable. While Aerosol® TR-70 (sodium bis(tridecyl) sulfosuccinate) could be used as an emulsifier to prepare polymer particles encapsulating Epolight™ 1117 tetrakis aminium dye, TR-70 only provided limited stabilization effects on the tetrakis aminium dye. Sodium dodecyl sulfate was found to have a better stabilizing effect on the Epolight™ 1117 during the emulsion and evaporation process, shifting retention in the particles from 50% retention, to up to 85-90% retention. Reducing the amount of SDS in the aqueous phase led to lower Epolight™ 1117 retention and larger particle size (Table 2).

TABLE 2
Stabilization effects of the surfactant type and quantity on
tetrakis aminium dye in aqueous phase during emulsification

| Surfactant in | | | | |
|---|---|---|---|---|
| aqueous phase | 0.6% TR-70 | 0.6% SDS | 0.4% SDS | 0.2% SDS |
| Median Particle size | 1.20 μm | 0.47 μm | 0.68 μm | 1.08 μm |
| % Epolight ™ 1117 | 51.70% | 82.96% | 80.17% | 74.97% |
| Retention | | | | |

The polymer used for this application is preferred to have a glass transition temperature significantly greater than the temperature of the environment for the intended use.

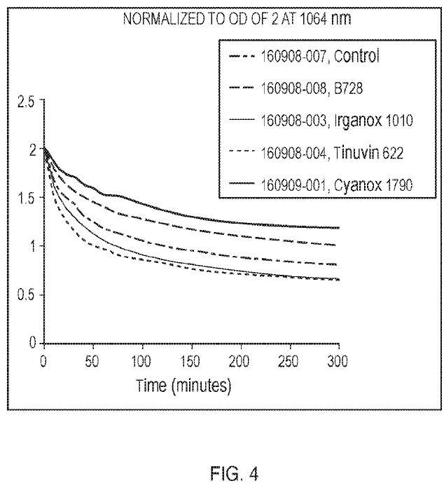

Various commercially available acrylic polymers were screened for preferred particle performance characteristic such as particle size distribution, IR dye stability and encapsulation efficiency. NeoCryl® B-851, a butyl acrylate/styrene copolymer proved to have a hydroxyl value too high, leading to a more polar particle and poor retention of the embedded tetrakis aminium dyes. NeoCryl® B-818, an ethyl acrylate/ethyl methacrylate copolymer, contained a lower hydroxyl value, but was still swellable in low molecular weight alcohols. NeoCryl® B-805, a methyl methacrylate/butyl methacrylate copolymer, had suitably a low hydroxyl value and a high Tg (99° C.) for human body applications. Use of a pure methyl methacrylate polymer, NeoCryl® B-728, led to greater degradation of the Epolight™ 1117 dye, as shown in FIG. 4.

The loading of dyes within the particles is as high as possible without degrading the cohesion of the polymer. The additives that stabilize the dye within the particles have been studied. The antioxidant Cyanox® 1790 was found to have a positive impact on dye stability.

### Example 2 (iii). Particle Size Determination

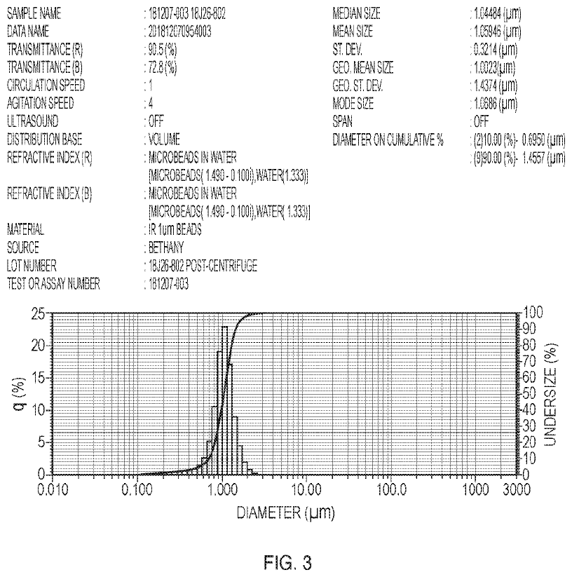

The particle size and size distribution of the NIR dye/MMA/BMA copolymer particles were measured by a Horiba LA-950 Particle Size Analyzer in distilled water with pH 7.4 (FIG. 3). All the particle size measurements were carried out at room temperature (17-23° C.).

Various additional Epolight™ 1117 particles are prepared according to the procedures set forth in the Example 1(i) above. The physicochemical properties of the resulting particles are summarized in Table 3 below.

TABLE 3
Particle Structure

| | | | particle size | polymer/dye | |
|---|---|---|---|---|---|
| | | polymer | range | weight ratio | |
| entry | IR dye | carrier | (micron) | range | additive |
| 1 | Epolight ™ | B805^{a} | 0.47, 0.68, | 4.4:1 | Cyanox ® |
| | 1117 | | 1.08, 1.20 | | 1790^{b} |
| | | | | | SDS^{c} |

^{a}Polymer B805 ®: copolymer of 96% methyl methacrylate and 4% butyl methacrylate.
^{b}Cyanox ®1790: dye stabilizer mixed in the polymer matrix.
^{c}SDS = sodium dodecyl sulfate, surfactant for emulsion solvent evaporation particle fabrication method.

### Example 2 (iv) Optical Properties of the Epolight™ 1117 IR Dye-B805 Particles

The optical properties of the Epolight™ 1117 IR dye-B805 particles dispersed in an aqueous water are determined by UV-VIS spectroscopy.

TABLE 4
Properties of Epolight ™ 1117 IR Dye

| | | Peak absorption | Extinction | |
|---|---|---|---|---|
| | Molecular | wavelength | coefficient | Non-cytotoxic |
| | Weight | (nm) | (M⁻¹*cm⁻¹) | concentrations |
| Dye | (g/mol) | (in DCM^{a}) | (in DCM) | (μM) |
| Epolight ™ | 1211 | 1098 | 105,000 | 32 |
| 1117 | | | | |

^{a}DCM is the abbreviation for dichloromethane.

### Example 2 (v): Preparation of the Biodegradable Particles

Poly(lactide-co-glycolide) (PLGA) (MW: 10,000-15,000 Da), Methoxy poly(ethylene glycol)-bpoly(lactide-co-glycolide) (mPEG-PLGA) (MW: 2-15 k.Da) are purchased from PolySciTech® (West Lafayette, Ind., USA). Epolight® 1117 was purchased from Epolin Inc (Newark, N.J., USA) and; ICG was purchased from AFG Biosciences (Northbrook, Ill., USA), IR-193 dye was a gift from Polaroid(Cambridge, Mass.) to Bambu Vault; All cell lines are obtained from ATCC (Manassas, Va.). The 3-(4,5-dimethylthiazol-2-yl)-5-(3-carboxymethoxyphenyl)-2-(4-sulfophenyl)-2H-tetrazolium (MTS) assay kit is purchased from Promega Corporation® (Madison, Wis., USA), Triton-X and other HPLC grade organic solvents are obtained from Fisher Scientific™ (Agawam, Mass., USA).

Multiple commercially available infrared dyes are screened to find a preferred composition to provide localized heat delivery to a tissue site with sufficient temperature rise to accelerate a reaction outside of the particle. The infrared dyes screened include ICG, IR-193 dye, Lumogen® IR 1050, Epolight® 1117, Epolight® 1125, and Epolight® 1178.

Amphiphilic co-polymers of PLGA and PEG are used to prepare PLGA/PLGA-PEG NPs with a blend of 75:25 of PLGA and PLGA-PEG. Epolight™ 1117 or ICG loaded NPs are synthesized by adding Epolight™ 1117 or ICG to the polymer solution containing blend of 75:25 of PLGA and PLGA-PEG. Similarly, empty NPs (without the IR dye) are prepared.

IR Dye concentration is measured by NIR spectrophotometry by measuring absorbance and using Beer's law to estimate concentration. Particle size, polydispersity index and zeta potential are confirmed by dynamic light scattering using a Zetasizer (ZS-90 from Malvern Instruments) and scanning/transmission electron microscopy. Encapsulation efficiency is calculated for the IR Dye by estimating the final amount of IR Dye in the purified particles (using concentration measured by UV spectrophotometry) and dividing that by amount that is originally used during the synthesis of the particles.

$$\% second material encapsulation efficiency = \frac{( Amount of the second material in mg added in the synthesis )}{( A mount of second material in mg that was added during synthesis )} \times 100 \%$$

### Example 3. Particle Characterization and Particle In Vitro Stability Study

### Example 3a. Particle Size Distribution for the Freshly Made Theragnostic Particles

Horiba® LA-950 Particle Size Analyzer in de-ionized water with pH 7.4 measures the particle size and size distribution. All the particle size measurements are carried out at 25° C. All the measurements are performed in triplicate.

### Example 3b In Vitro Stability Study

The in vitro stability of the particles loaded with squaraine dye, Epolight® 1117, or ICG dye is investigated by storing the formulation at 4° C. and in media containing 10% FBS at 37° C. to monitor their stability for shelf life (over weeks and months) and at physiologically relevant temperature. Size, zeta potential, polydispersity indices are measured at different time points using the Zetasizer (ZS-90).

### Example 4. Extractable Particle Content Test

UV/VIS/NIR: The absorbance spectrum for the biosensor and the second material is measured using Shimadzu® UV-3600 UV-NIR Spectrophotometer.

### (a) The Percentage of Loading Determination

The percentage of the second material or the biosensor loaded in to the particles can be determined according to the following procedure: Dried particles are ground in a mortar and pestle and 5-10 milligrams of the ground particles are added to 25 mL of dichloromethane (DCM). The UV-VIS-NIR absorbance spectrum of the leached IR dye is measured using Shimadzu® UV-3600 UV-VIS-NIR Spectrophotometer. The concentration of the extracted drug in DCM is determined from application of Beer's law.

$$[ Material ] ( μ M ) = \frac{{Absorbance}_{λ}}{{ɛ}_{λ} \times l} \times 1 {0}^{6}$$

where the path length, l, is 1 cm.

The quantity of the second material or the biosensor (the material) extracted is determined from the product of the concentration, the amount of total DCM solution (25 mL), and the molecular weight of the material. The material loading as a percentage of the total particle mass is determined from:

$$Material Loading ( \% ) = \frac{Amount of material in DCM solution}{Amount of micro particle used} \times 100 \%$$

### (b) Surfactant-Based Extractable Test: The Determination of the Percentage of the Material Leached (Standard Protocol)

Dried particles (50 mg) are added to 3 mL of 1% sodium dodecyl sulfate to form a dispersion. The dispersion is sonicated for about 1 hour. The dispersion is centrifuged, and the supernatant component is withdrawn and filtered through a 0.2 μm syringe filter. The UV-VIS absorbance spectrum of the filtrate is measured using Shimadzu® UV-3600 UV-VIS-NIR Spectrophotometer in a 1 cm cell.

The amount of the dye leached is calculated as in 3(a) above by applying Beer's law.

### Example 5. Efficacy Determination Protocol

An Efficacy Determination Protocol is used to evaluate the effect of biological chemicals including bodily fluid on the biosensor in the diagnostic particle or the second material in the theragnostic particle that is encapsulated inside the particle. Briefly, a known quantity of the diagnostic or theragnostic particles containing the biosensor or the second material is incubated with 1 mL of complete cell culture media (for example macrophage or neutrophil cell growth media) containing 10% fetal bovine serum at 37° C. As a negative control, the same quantity of the diagnostic or theragnostic particles containing the material is suspended in 1 mL of distilled water and incubated at 37° C. At different time intervals (for example: 24 h, 48 h, 72 h, 120 h) following incubation, for both the test and control, a small portion of the sample is removed and diluted with distilled water. If the biosensor or the second material absorbs UV-VIS-IR, then the UV-VIS-IR absorbance spectrum of each solution is measured using a UV-VIS-IR spectrophotometer. Degradation of the biosensor or the second material by the cell culture medium is determined by comparing the peak absorption in the spectrum of the test sample to the absorption of the control sample at the same spectral peak, and degradation is generally reported as the percentage in the reduction in the peak absorbance. If the biosensor or the second material does not absorb UV-VIS-IR, other analytical tools, like NMR, HPLC, LCMS etc., would be used to quantify the concentration of the biosensor or the second material in the test and control. In some instances, if the degradation of the biosensor or the second material is less than 90% after being subject to the body chemicals, then the particle is considered passing the Efficacy Determination Protocol.

### Example 6. Extractable Cytotoxicity Test

100 mg of the diagnostic or theragnostic particles are weighed out and then suspended in 1 mL of cell culture media Dulbecco's Modified Eagle's medium (DMEM) containing 10% (fetal bovine serum) FBS and vortexed five times to ensure thorough mixing. This suspension is then incubated at 37° C. in an incubator for 24 hrs. After the incubation period is complete, the suspension is centrifuged at 10,000 g for 10 minutes and the supernatant is collected. The supernatant solution is then filtered through a 0.1-micron syringe filter and is used for cytotoxicity evaluation as the “neat” or 1× sample. This 1× neat extract is serially diluted with media containing 10% FBS for cytotoxicity testing. The following serial dilutions were made using the neat extract and the DMEM supplemented with 10% FBS: 2× (2-fold dilution), 4× (4-fold dilution), 8× (8-fold dilution), 16× (16-fold dilution) and 32× (32-fold dilution), 64× (64-fold dilution) and 128× (128-fold dilution).

Inhibitory Concentration for 30% cell killing (IC₃₀) of the extract on HepaRG is determined by performing an MTS assay, a standard colorimetric method to measure the cell viability following incubation with different dilutions of the 1× extract obtained above. HepaRG cells are plated in a 96-well culture plate at a density of 10,000 cells per well and allowed to adhere to the surface overnight. Extract concentrations ranging from 1× to 128× are added and incubated for 24 hours at 37° C., in a 5% CO₂ incubator. Controls for the cytotoxicity experiment include “live” and “dead” (cells that are killed due to osmotic pressure by adding D.I. water). “Live” cells have nothing except cell culture media containing 10% FBS added to them and are used to obtain the 100% viability data point. The “dead” control is used to obtain the 0% viability data point for calculating the % viability of cells that are incubated with the different extract concentrations. After 24 hours, to a final volume of 100 μL of media in the cells, 20 of PMS activated MTS reagent is added and incubated for 90 minutes. The absorbance is measured at 490 nm using a plate reader (Spectramax M2e, Molecular Devices). Viability of cells is calculated using the absorbance measured at 1× dilution of the extract and the results of absorbance for serial dilutions 1× to 128× of the extract are plotted in MS Excel using linear regression curve fitting algorithm to obtain the IC₃₀. All the samples are tested in triplicate and results are averaged over the three repeats. A particle that results in a 70% cell viability in the cytotoxicity test is considered passing the Extractable Cytotoxicity Test.

### Example 7. Thermal Cytotoxicity Test

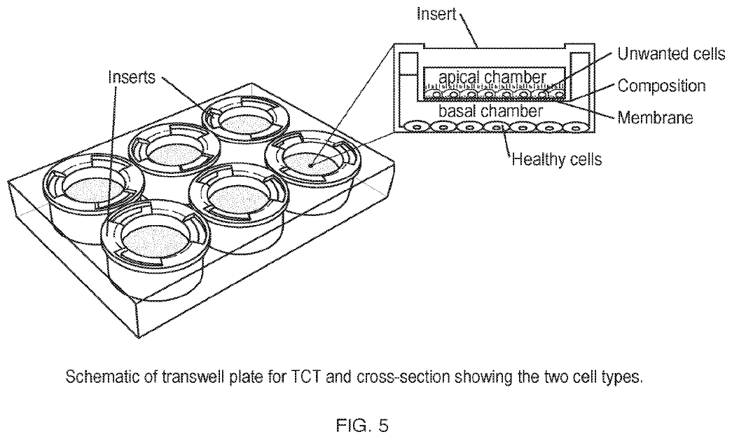

The thermal cytotoxicity test uses the 24-well Corning Transwell™ Multiple Well Plate with Permeable Polycarbonate Membrane Inserts. Normal epithelial cells, FHC (ATCC® CRL-1831™) obtained from ATCC, are plated in these 24-well culture plates at a density of 30,000 cells per well and allowed to adhere to the plate surface overnight. Microbial cells (Staphylococcus Aureus) are seeded at a density of 30,000 cells and grown on the transwell inserts of the 24-well Corning plate. The following day, the media in each well is replaced with fresh, cell growth media containing 10% fetal bovine serum. A CellCrown™ insert is used to expose the microbial cells to the particles at different concentrations for testing the thermal cytotoxicity on the microbial cells. These are placed into the trans-well of the Corning plate, such that the insert is submerged in the media but not directly in contact with the microbial cells. The particles to be irradiated are mixed with cell culture media and added on to the CellCrown™ insert (which includes a transparent PET filter with a pore size of 0.5 μm, allowing heat to easily spread out of the filter into the surrounding media). The CellCrown™ inserts are removed 1 h after incubation of the microbial cells with the theragnostic particles and media in the transwell is replaced with fresh complete cell growth media. The incubation period allows for the uptake of the theragnostic particles by the microbial cells. The microbial cells are then exposed to the exogenous source. This will include irradiation with a laser at three different fluences, each at three different pulse durations to ensure the heat generated is going to kill at least 70% of the microbial cells at different particle concentrations and light doses. The transwell inserts that have the microbial are removed 1-h after irradiation with the exogenous source and placed in a regular 24-well plate for determining the number of microbial cells killed by laser irradiation using an MTS assay, a standard colorimetric method to measure the cell viability 24 h after the irradiation. The non-diseased/normal cells are also incubated for an additional 23 hours at 37° C., in a 5% CO₂ incubator. The viability of the non-diseased, normal cells following the irradiation is also determined by performing an MTS assay to measure the cell viability 24 h after the irradiation. Controls for the thermal cytotoxicity experiment included “live”, “dead” (cells were killed due to osmotic pressure by adding D.I. water) and the particles alone, (i.e. with no laser irradiation) and “light only” for each of the two cell types used. “Live” cells will have nothing except cell culture media containing 10% FBS added to them and are used to obtain the 100% viability data. The “dead” control is used to obtain the 0% data point. “Light only” control includes exposing cells to the equivalent light dose without the composition present in the well. Light doses will be selected to ensure little to no killing of cells is observed using the light only control. At the end of the 24 hours, to a final volume of 200 μL of media in the wells, 40 μL of PMS activated MTS reagent is added and incubated for 90 minutes. The absorbance is measured at 490 nm using a plate reader (Spectramax M2e, Molecular Devices). Viability of both the cell types is calculated using the absorbance measured and the results plotted in MS Excel. The composition and light dose(s) that do not kill any more than 30% of the non-diseased cells but kill at least 70% of the diseased cells are considered passing the thermal cytotoxicity test, as shown in FIG. 5.

### Example 8. Material Process Stability Test on Theragnostic Particles

Theragnostic particles containing the second material are dispersed in a 2% solution of gelatin in warm water. The suspension is vortexed and transferred to 50 mm plastic culture dishes and allowed to gel, producing a greenish gel. The optical density is measured by reflectance spectroscopy to provide a baseline absorbance.

Areas on the culture dishes are irradiated over a range of pulse widths and fluences that span the conditions expected for use. Generally, pulse widths range from about 100 μs to about 1 second, with fluences that range from about 0.1 J/cm² to about 60 J/cm². The absorbance of the second material is measured for each exposure condition and compared to the baseline absorbance. The preservation greater than 50% absorbance of the second material after subject to such process conditions is considered to pass the Material Process Stability Test.

### Example 9. Controlled Heat Generation from Laser-Excited Particle Heaters in Gelatin

The test is to determine threshold conditions for controlled heat generation that produces a thermal increase to 50° C. Heat was generated by exposing a gelatin gel suspension of IR dye particle as in Example 1(ii) above with a red thermochromic pigment with 50° C. thermal threshold for color loss to laser irradiations with various operating parameters. The gelatin is a degradation product from collagen. The collagen is the primary extracellular matrix protein. The gelatin medium in this example mimics the soft tissue at the infection site. The dye particle as in Example 1(ii) above and the biosensors or the diagnostic particles disclosed herein can be combined to form theragnostic formulations that is useful for detecting the drug resistant bacteria and provide guidance to destruction of them via hyperthermia induced by the remotely-triggered activation of the IR dye particles.

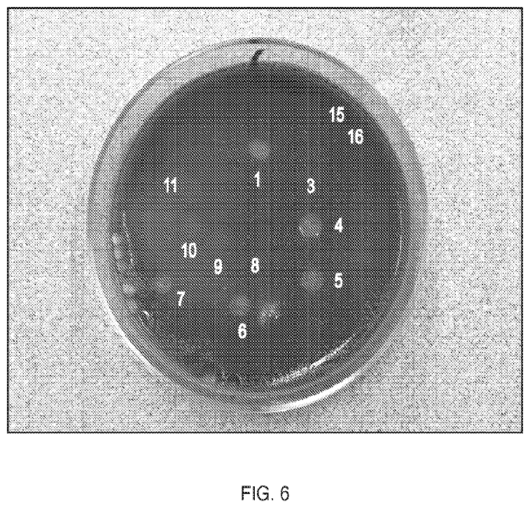

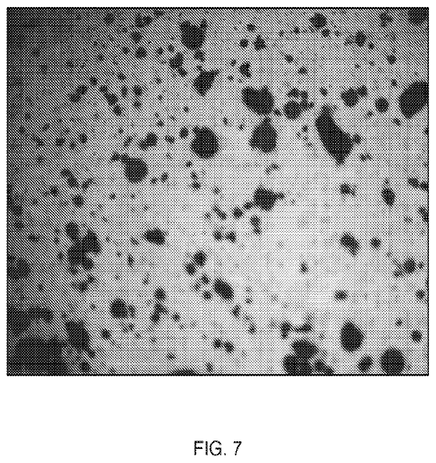

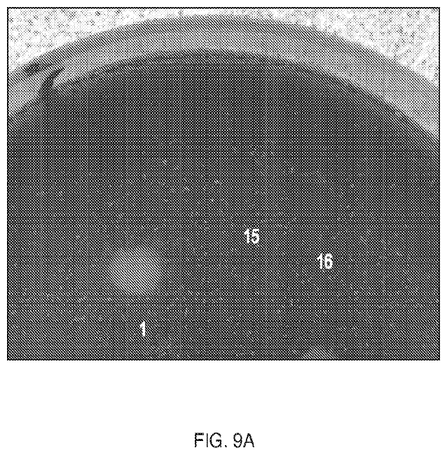

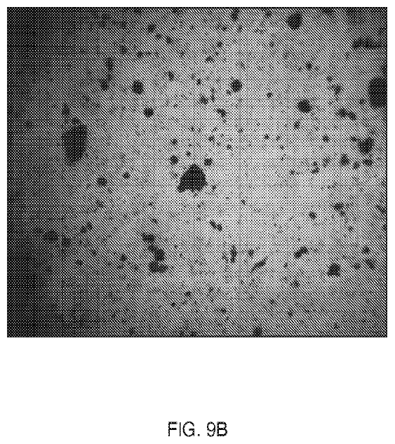

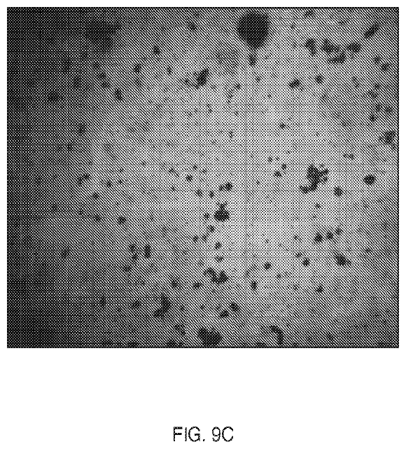

The results of the tests as summarized in the table below demonstrated the capability of the IR dye particles to absorb energy from laser irradiation and converts the photonic energy into heat. Under the laser operating parameters as set forth below, the heat traveled outside the particle and induced a localized hyperthermia in area surrounding the IR dye particle heaters (see FIGS. 6-9, Table 5).

Thermochromic MC Pigment 50° C. Red (a red thermochromic dye with a threshold temperature for color loss at 50° C., TM PD 50 3111, Lot #MC1204191) was purchased from Sandream Enterprises. Unflavored, commercial, food grade Knox® gelatin was used as received.

A 2.0 wt. % stock solution of gelatin in water was prepared by wetting one gram gelatin with 12 g of cold water, then adding 37 g of water at 75° C., and stirring until dissolved. A 30.0 wt. % stock suspension of particle heaters in water was prepared by suspending of 3.0 g of the particles from Epolight™ 1117 IR dye particles in 7.0 mL of water.

To 65.0 mg of the particle heater suspension in a 4 dram glass vial was added 25 mg of red thermochromic pigment to form a mixture. To this mixture was added 2.0 g of the 2% gelatin solution, and the glass vial was vortexed for 5 minutes and set aside for use.

The vortexed suspension was transferred by pipette to a 50 mm plastic culture dish, spread evenly, and allowed to cool to form a gel. The particle heaters were spread uniformly within the gelatin gel matrix and gave a greenish color. The particles of the red thermochromic pigment were distributed unevenly within the gelatin matrix (see FIG. 6).

A control sample of red thermochromic pigment, but lacking the particle heaters, was also prepared using the procedure described above by suspending 25 mg of dye in 2 g of 2% gelatin solution, vortexing, spreading evenly in a 50 mm plastic culture dish and allowing to gel.

After the gel had set, it was irradiated with a laser under a variety of different operating parameters. Several regions of the gel (spots 1-3) were first irradiated at 1064 nm in spots of 5 mm diameter with a Lutronic solid state laser, with exposures of 3.51 J/cm² using a 10 ns pulse (Q-switched mode) (Spot 1) and of 2.01 J/cm² (Spot 2) and 3.51 J/cm² (Spot 3) using a 350 μs pulse (Spectra mode). A second set of regions (spots 8-16) were irradiated at 980 nm in spots of about 3 mm diameter with a 10 Watt, electrically switched, CW semiconductor laser with pulse widths ranging from 10-250 ms and delivered energies ranging from 0.5-5 J. The color change effects caused by the laser exposures were photgraphically recorded using an iPhone camera or microscope camera. The visual results of color changes are shown in FIGS. 6-9. These experiments are summarized in Table 5.

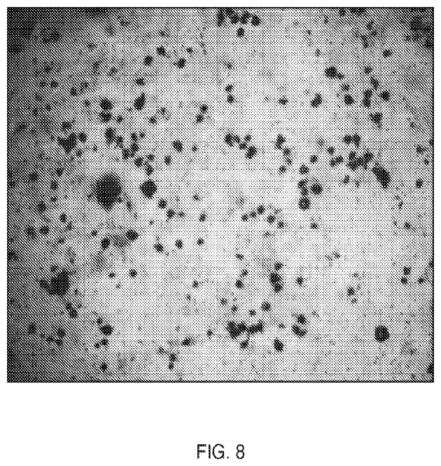

TABLE 5
Results of Laser Exposure of Particle Heaters
and Thermochromic Pigment in Gelatin

| | | Pulse | Fluence, | | |
|---|---|---|---|---|---|
| Spot | Laser | width | J/cm² | Result | Image |
| 1 | Lutronic | 10 ns | 3.51 | White spot, | |
| | (1064 nm) | | | red pigment | |
| | | | | decolorized, IR | |
| | | | | dye color gone | |
| 2 | Lutronic | 350 μs | 2.01 | Minimal disturbance | |
| | (1064 nm) | | | of gelatin | |
| 3 | Lutronic | 350 μs | 3.51 | Slight depression in | |
| | (1064 nm) | | | gelatin, IR dye | |
| | | | | not changed. | |
| | | | | Red pigment melted | |
| | | | | and color gone. | |
| 8 | Semiconductor | 200 ms | 28.3 | A spot was formed | |
| | laser (980 nm) | | | in the gelatin. IR | |
| | | | | dye was not | |
| | | | | changed, but | |
| | | | | red pigment | |
| | | | | appeared to be melted | |
| | | | | and color gone. | |
| 9 | Semiconductor | 2 × | 70.7 | Same as spot 8 but | FIG. 8 |
| | laser (980 nm) | 250 ms | | bigger spot | |
| 10 | Semiconductor | 250 ms | 35.4 | Same as spot 8 but | |
| | laser (980 nm) | | | slightly bigger spot | |
| 11 | Semiconductor | 100 ms | 14.1 | Approximately 3 | |
| | laser (980 nm) | | | mm spot, surface | |
| | | | | particles of red | |
| | | | | pigment mostly gone | |
| 12 | Semiconductor | 50 ms | 7.1 | Same effect on gelatin, | |
| | laser (980 nm) | | | smaller spot, surface | |
| | | | | particles of red | |
| | | | | pigment evident | |
| 13 | Semiconductor | 10 ms | 0.7 | Minimal disturbance | |
| | laser (980 nm) | | | of gelatin observed | |
| 14 | Semiconductor | 30 ms | 2.1 | Slight “melting” | |
| | laser (980 nm) | | | of gelatin | |
| 15 | Semiconductor | 7 × | 14.9 | Similar to spots 11 | FIG. |
| | laser (980 nm) | 30 ms | | and 16. Slightly | 9B |
| | | | | smaller spot | |
| | | | | than 16 but | |
| | | | | red pigment melted | |
| | | | | and color gone | |
| 16 | Semiconductor | 200 ms | 14.1 | Similar to spot 15 but | FIG. |
| | laser (980 nm) | | | larger spot. Red | 9C |
| | | | | pigment melted, | |
| | | | | color gone. | |

The results in Table 5 show that 1064 nm Q-switched laser irradiation of 3.51 J/cm² led to significant loss of IR dye and decolorization of red thermochromic pigment. Irradiation with a similar fluence but longer pulse width (Spectra mode) does not show IR dye degradation but does show melting and decolorization of the red thermochromic pigment. Reducing the fluence to 2.01 J/cm² led to no decolorization and little evidence of heat generation as evidenced by distortion of the gelatin.

Irradiation using the semiconductor laser at 980 nm required greater fluence to produce an equivalent decolorization of the thermochromic pigment. For example, a dose of 14 J/cm² was required to demonstrate complete loss of red color; lower fluence led to no or minimal observable effect. In all cases with this laser, no loss of IR dye was observed. The retention of the IR dye was evidenced by the ability to provide enough energy to decolorize the red pigment using several sequential with lower energy pulsed to achieve the same result as irradiation with a single pulse of equivalent total fluence.

The control sample, with red thermochromic pigment only, showed no change when exposed to the semiconductor laser using the settings described in Table 5.

### Example 10. Photothermal Antibacterial Property of the Particle Heaters

Two representative bacteria types including Gram-negative E. coli and Gram-positive S. aureus are utilized as model cells. The antibacterial effects of the theragnostic particles and theragnostic composition for both cell types are determined under NIR irradiation.

The photothermal antibacterial experiments are conducted in a 10-mL transparent glass bottle. Bacterial cells are suspended in the glass bottle containing 4.5 mL of sterilized physiological saline and then 0.5 mL of PLGA/PLGA-PEG encapsulated IR dye particle suspension is added into the saline. The concentrations of bacterial cells and the heat delivery particles are controlled at 2×10⁶ CFU/mL and 50 mg/L, respectively. The glass bottle is then placed under NIR laser irradiation (805 nm, 808 nm or 1064 nm, 0.028 W/cm²) and pulsed at 400 ms interval at a distance of 7 cm for 5 to 10 minutes. At different time intervals, 100 μL of bacteria-particle heater mixture is sampled, diluted, and measured for surviving bacterial concentration using the plate count method. Control experiment is carried out with 5 mL of bacteria suspension (2×10⁶ CFU/mL) in the absence of heat delivery particles both with and without NIR laser irradiation, and PBS buffer with heat delivery particle. Bacteria killing rate, irradiation duration curve at the near infrared irradiation at 1064 nm (0.028 W/cm²) of a 10⁸ CFU/mL bacteria sample, measure the photothermal antibacterial property. The duration of irradiation at a near infrared light is gradually increased from 0 minute to 10 minutes.

While the concepts of the present technology have been particularly shown and described above with reference to exemplary embodiments thereof, it will be understood by those of ordinary skill in the art, that various changes in form and detail can be made without departing from the spirit and scope of the concepts described herein. It is to be understood that features from any one embodiment described herein may be combined with features of any other embodiment described herein to form another embodiment of the invention.

1. A biosensor for the detection of drug resistant bacteria comprising:
a first material FG responsive to an antibacterial inactivating factor secreted by the drug resistant bacteria; and
a spectroscopic probe D,
wherein FG is coupled to the spectroscopic probe, wherein FG masks the activity of D,
wherein the antibacterial inactivating factor causes FG to decouple from D, resulting in a detectable optical response.

2. A biosensor of claim 1, wherein D is selected from the group consisting of a fluorophore, a chromophore, an infrared chromophore, a visible light chromophore, and combinations thereof.

3. The biosensor of any one of claim 1-2, wherein the antimicrobial inactivating factor is selected from the group consisting of β-lactamase, penicillinases, cephalosporinases, carbenicillinases, oxacillinases, carbapenemases including the metallo-β-lactamases, and extended spectrum β-lactamases, erythromycin (macrolide) esterase, chloramphenicol (phenicol) hydrolase, and combinations thereof.

4. A biosensor of any one of claim 1-3, wherein FG comprises a fragment derived from a β-lactam antibiotic, macrolide antibiotic, or amphenicol antibiotic.

5. A biosensor of claim 4, wherein the β-lactam antibiotic is selected from the group consisting of benzylpenicillin, phenoxymethylpenicillin, propicillin, pheneticillin, azidocillin, clometocillin, penamecillin, cloxacillin, dicloxacillin, flucloxacillin, oxacillin, nafcillin, methicillin, amoxicillin, ampicillin, pivampicillin, hetacillin, bacampicillin, metampicillin, talampicillin, epicillin, ticarcillin carbenicillin, carindacillin, temocillin, piperacillin, azlocillin, mezlocillin, mecillinam, pivmecillinam, sulbenicillin, faropenem, ritipenem, ertapenem, antipseudomonal, doripenem, imipenem, meropenem, biapenem, panipenem, cephalothin (also known as cefalotin), cefazolin, cefazaflur, cefalexin, cefadroxil, cefapirin, cefazedone, cefazaflur, cefradin, cefroxadin, ceftezole, cefaloglycin, cefacetril, cefalonium, cefaloridin, cefalotin, cefalonium, cefapirin, cefatrizine, cefazedon, cefaclor, cefotetan, cephamycin, cefoxitin, cefprozil, cefuroxime, axetil, cefamandole, cefminox, cefonicid, ceforanide, cefotiam, cefbuperazone, cefuzonam, cefmetazole, carbacephem, loracarbef, cefixime, ceftriaxone, antipseudomonal, ceftazidime, cefoperazone, cefdinir, cefcapene, cefdaloxime, ceftizoxime, cefmenoxime, cefotaxime, cefpiramide, cefpodoxime, ceftibuten, cefditoren, cefetamet, cefodizime, cefpimizole, cefsulodin, cefteram, ceftiolene, oxacephem, flomoxef, latamoxef, cefozopran, cefpirome, cefquinome, ceftaroline, fosamil, ceftolozane, ceftobiprole, ceftiofur, cefquinome, and cefovecin.

6. A biosensor of claim 4 or 5, wherein the β-lactam antibiotic is derived from the cephalosporin class of antibiotics.

7. A biosensor of claim 4 or 5, wherein the FG comprises a fragment having 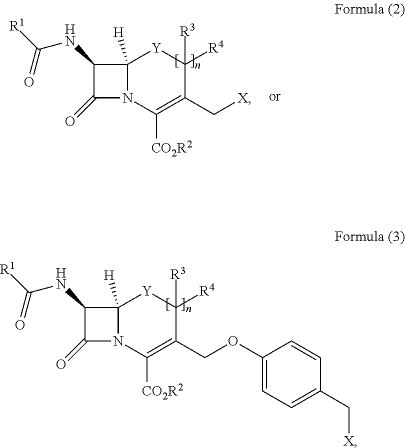
wherein R¹ is selected from the group consisting of —CH₂—CN, —CH₂—S—CH₂—CN, —CH₂—CF₃, —CH₂—CHF₂, —CH₂—O-Ph, —CH(-Me)(—O-Ph), —CH(-Et)(O-Ph), —CH₂-Ph, 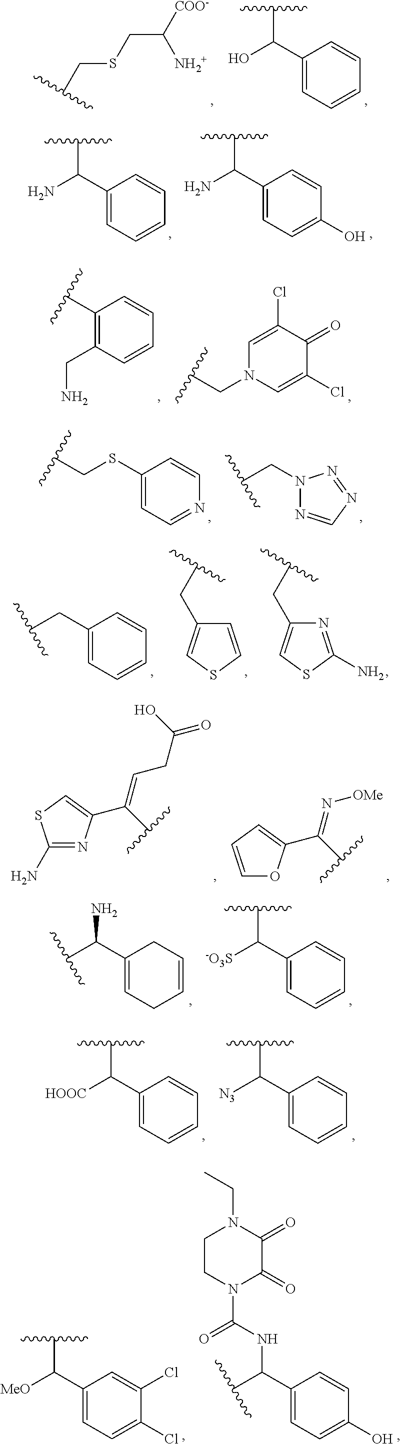 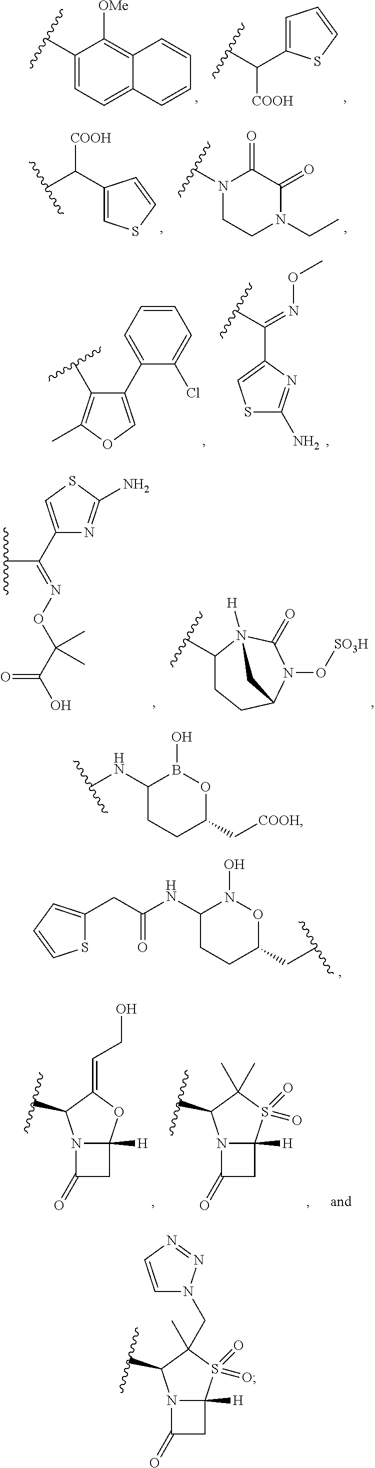
R², R³, and R⁴ are each independently selected from the group consisting of H, substituted and unsubstituted C₁-C₁₂ alkyl group, substituted and unsubstituted C₁-C₁₂ alkenyl group, substituted and unsubstituted C₁-C₁₂ alkynyl group, and substituted and unsubstituted aryl group;
Y is a bond, S, or O; and
X represents the point of attachment to the spectroscopic probe D.

8. A biosensor of claim 4, wherein the FG comprises a fragment having 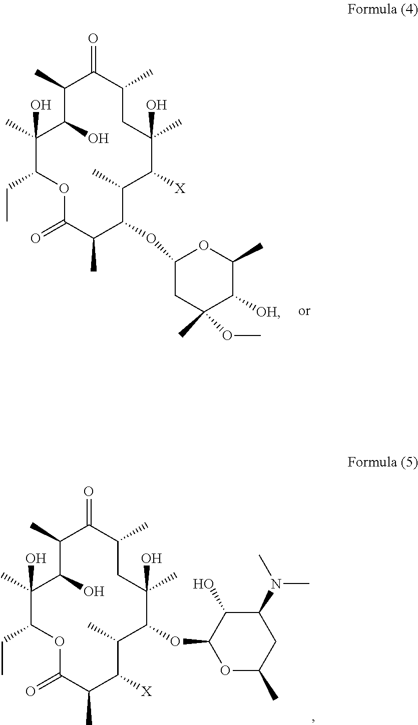
wherein X represents the point of attachment to the spectroscopic probe D.

9. A biosensor of claim 4, wherein the FG comprises a fragment having 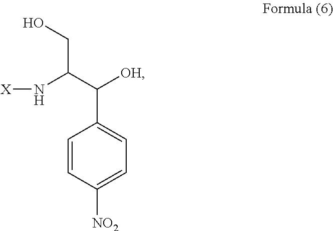
wherein X represents the point of attachment to the spectroscopic probe D.

10. A biosensor of claim 1, wherein FG is derived from 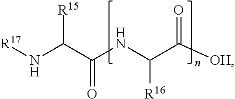
wherein the biosensor is sensitive to peptidase secreted by the drug resistant bacteria.

11. A biosensor of claim 1, wherein FG is 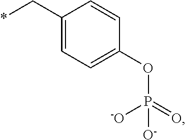
wherein the biosensor is effective for detecting bacteria secreting phosphatase.

12. A biosensor of claim 1, wherein FG is 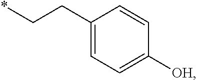 [SMILES: *CCc1ccc(O)cc1]
wherein the biosensor is effective for detecting bacteria secreting tyrosinase.

13. A biosensor of claim 1, wherein FG is 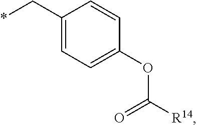
wherein the biosensor is effective for detecting bacteria secreting esterase.

14. A biosensor of claim 1, wherein FG is selected from the group consisting of 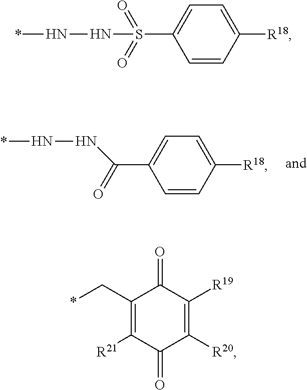
wherein the biosensor is sensitive to redox microenvironment surrounding bacteria.

15. A biosensor of claim 1, wherein the optical response produced by the biosensor is a color change from colored state to colorless state.

16. A biosensor of claim 1, wherein the optical response produced by the biosensor is a color change from colorless state to colored state.

17. A biosensor of claim 1, wherein the optical response produced by the biosensor is a change from non-fluorescent state to fluorescent state.

18. A biosensor of claim 1, wherein D comprises a structure derived from a xanthene chromophore or a triarylmethane chromophore.

19. A biosensor of claim 18, wherein D comprises a structure derived from a fluorescein, a rhodol, or a rhodamine.

20. A biosensor of claim 16, wherein D is a colorless component derived from a leuco dye, wherein reaction of the biosensor with the inactivating factor produces a fluorescein, a rhodol, or a rhodamine chromophore.

21. A biosensor of claim 19, wherein D comprises a colored component having

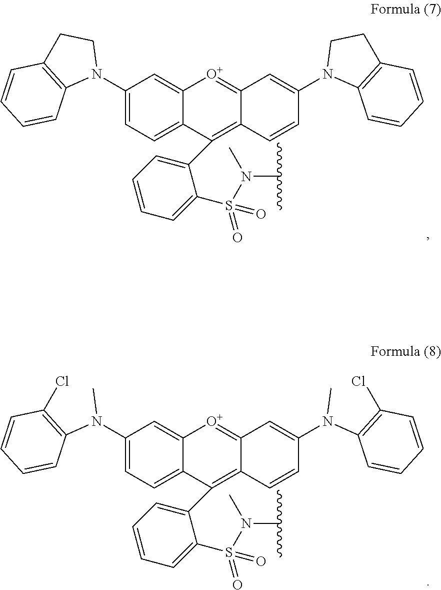 [SMILES: *N(C)S(=O)(=O)c1ccccc1-c1c2ccc(N3CCc4ccccc43)cc2[o+]c2cc(N3CCc4ccccc43)ccc12 ; *N(C)S(=O)(=O)c1ccccc1-c1c2ccc(N(C)c3ccccc3Cl)cc2[o+]c2cc(N(C)c3ccccc3Cl)ccc12]

22. A biosensor of claim 1, wherein D comprises a colorless leuco dye component having 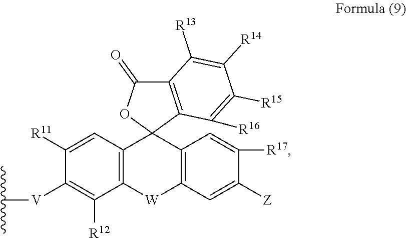
wherein
W is O, N, S or —CH₂—;
Z is —NR⁹R¹⁰, —O—CH₂-Ph, or V;
R⁹ and R¹⁰ is a substituent each independently selected from the group of H, substituted and unsubstituted C1-C12 alkyl group, substituted and unsubstituted C1-C12 alkenyl group, substituted and unsubstituted C1-C12 alkynyl group, substituted and unsubstituted aryl group, fluoroalkyl, substituted and unsubstituted carbocyclyl, substituted and unsubstituted carbocyclylalkyl, substituted and unsubstituted aralkyl, substituted and unsubstituted heterocycloalkyl, substituted and unsubstituted heterocycloalkylalkyl, substituted and unsubstituted heteroaryl, and substituted and unsubstituted heteroarylalkyl;
R¹¹, R¹², R¹³, R¹⁴, R¹⁵, R¹⁶, and R¹⁷ is each independently selected from the group of H, Cl, F, Br, CN, NO₂, —NR⁹R¹⁰, C1-C6 alkyl group, and C1-C6 alkoxyl group; and
V represents the point of attachment of the fragmentable group FG.

23. A biosensor of claim 1, wherein D comprises a structure derived from a thiazine, an oxazine, or a phenazine chromophore.

24. A biosensor of claim 23, wherein D comprises a colorless leuco dye component having 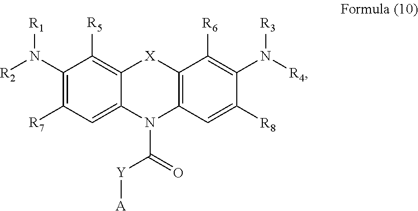
wherein
X is —CH₂, O, N, or S;
Y is a bond, O or N;
R¹, R², R³, R⁴ are each independently selected from the group consisting of H, substituted and unsubstituted C₁-C₁₂ alkyl group, substituted and unsubstituted C₁-C₁₂ alkenyl group, substituted and unsubstituted C₁-C₁₂ alkynyl group, and substituted and unsubstituted aryl group;
R⁵, R⁶, R⁷, and R⁸ is each independently selected from the group consisting of H, Cl, F, Br, CN, NO₂, —NR⁹R¹⁰, C₁-C₆ alkyl group, and C₁-C₆ alkoxyl group; and
A represents the point of attachment of the fragmentable group FG.

25. A biosensor of claim 24, wherein D comprises a structure derived from methylene blue.

26. The biosensor of any one of the claims 1-25, further comprising a solid support, wherein the R¹ of the β-lactam component is covalently bonded to the solid support.

27. The biosensor of any one of the claims 1-25, further comprising a solid support, wherein the spectroscopic probe is covalently bonded to the solid support.

28. The biosensor of any one of the claim 26 or 27, wherein the solid support is selected from the group consisting of a particle, fiber, electrospun nanofiber, a microgel, a wound dressing, a catheter, a membrane, a resin, a sponge, a paper, a cellulose filter paper, a sheet, a suture, an implant scaffold, a stent, a swab, a hydrogel, a film, a patch, a tape, a woven fabric, and a nonwoven fabric.

29. The biosensor of claim 28, wherein the solid support is a particle.

30. The biosensor of claim 28, wherein the solid support is a microgel comprising a dendritic polymer.

31. A molecular library comprised of biosensors of claim 1 that can detect specific bacterial pathogens.

32. A library of claim 31, wherein the elements of the library are based on a platform containing other leuco dyes including but not limited to, spiropyran, quinone, thiazine, phenazine, oxazine, pthalide-type, triarylmethanes, fluoran, and tetrazoliums.

33. A library of claim 31, wherein the elements of the library are based on a platform containing naturally occurring dyes including but not limited to, curcumins, hypericin, carotenes, anthocynanins, and any other phytochemical dyes.

34. A library of claim 31, wherein the elements of the library are based on a platform containing synthethic dyes that are not be leuco dyes azo dyes, xanthenes, phthalides and azomethine dyes.

35. A method for detecting the presence or absence of a drug resistant bacteria in a sample, the method comprising:
providing the biosensor of claim 1, and
contacting the biosensor with the sample, the biosensor showing the presence or absence of an optical response in the sample, wherein the presence of the optical response indicates the presence of the drug resistant bacteria.

36. The method of claim 35, wherein the optical response is a color change within the visible region of the electromagnetic spectrum.

37. The method of claim 35, wherein the optical response is fluorescence.

38. The method of claim 35, further comprising a step of quantifying the optical response using spectroscopy for determining the bacterial burden to inform antibiotic selection and dosage thereof.

39. A diagnostic composition for detecting drug resistant bacteria comprising at least one biosensor of claim 1.

40. The diagnostic composition of claim 39, wherein the spectroscopic probe, when released from the biosensor, gives a discrete color having the absorption wavelength in the visible light spectrum ranging from 400 nm to 500 nm.

41. The diagnostic composition of claim 39, wherein the spectroscopic, when released from the biosensor, probe gives a blue color having the absorption wavelength in the visible light spectrum ranging from 600 nm to 700 nm.

42. The diagnostic composition of claim 39, wherein the spectroscopic probe, when released from the biosensor, gives a discrete red color having the absorption wavelength in the visible light spectrum ranging from 400 nm to 600 nm.

43. A diagnostic composition of claim 39 comprising:
two or more biosensors of claim 1,
wherein each biosensor independently has a masked spectroscopic probe giving a discrete color after decoupling from an FG to produce a detectable optical response.

44. A diagnostic composition of claim 39 comprising:
three biosensors of claim 1;
wherein the diagnostic composition comprises three different populations of biosensors each independently having a cyan, magenta and yellow color to give a visible black color;
wherein the biosensors in the diagnostic composition each produces a detectable optical response from colored state to colorless state;
wherein the diagnostic composition turns to either of the primary color cyan, magenta and yellow after any two of the sensors being rendered colorless by the uncoupling of D from FG by the antibiotic inactivating factors.

45. The diagnostic composition of claim 44, wherein the two or more colors of the two or more biosensors are selected to give a mixed color having sufficient difference such that each of the two colors are visually discernable by naked eye.

46. The diagnostic composition of claim 44, wherein the spectroscopic probe, when released from the biosensor, gives a discrete color having the absorption wavelength in the visible light spectrum ranging from 400 nm to 500 nm.

47. The diagnostic composition of claim 44, wherein the spectroscopic probe, when released from the biosensor, gives a discrete blue color having the absorption wavelength in the visible light spectrum ranging from 500 nm to 600 nm.

48. The diagnostic composition of claim 44, wherein the spectroscopic probe, when released from the biosensor, gives a discrete red color having the absorption wavelength in the visible light spectrum ranging from 600 nm to 700 nm.

49. The diagnostic composition of claims 39-48 that is used for a sanitization.

50. The diagnostic composition of claims 39-48 that is used as a hand sanitizer.

51. A diagnostic particle for microbial detection comprising:
(1) a carrier; and
(2) a biosensor of claim 1.

52. The diagnostic particle of claim 51, wherein the particle is structured such that it passes the Extractable Cytotoxicity Test.

53. The diagnostic particle of claim 51, wherein the particle is structured such that it passes the Efficacy Determination Protocol.

54. The diagnostic particle of any one of claims 51-53, wherein the particle further comprises a shell to enclose the particle to form a core-shell particle.

55. The diagnostic particle of claim 54, wherein the shell comprises a crosslinked inorganic polymer selected from the group consisting of mesoporous silica, organo-modified silicate polymer derived from condensation of organotrisilanol or halotrisilanol, and combinations thereof.

56. A theragnostic particle comprising the diagnostic particle of claim 51-55,
wherein the diagnostic particle further comprises a second material interacting with an exogenous energy source to form the theragnostic particle.

57. The theragnostic particle of claim 56, wherein the theragnostic particle further passes the Thermal Cytotoxicity Test.

58. The theragnostic particle of claim 56, wherein the exogenous energy source is selected from the group consisting of an electromagnetic radiation, an electrical field, a microwave, a radio wave, an ultrasound, a magnetic field, and combinations thereof.

59. The theragnostic particle of claim 56, wherein the theragnostic particle structured to maintain its integrity or alters its structure after its exposure to the exogenous energy source.

60. The theragnostic particle of claim 56, wherein the theragnostic particle is porous, wherein the pores of the particle is plugged with a peptide degradable by the enzymes secreted by the microbes.

61. The theragnostic particle of any one of claim 56, wherein the shell comprises a plasmonic absorber selected from the group consisting of a thin film of noble metals including gold (Au), silver (Ag), copper (Cu), nanoporous gold thin film, and combinations thereof.

62. The theragnostic particle of claim 56, wherein the particle further comprises a coating made of polydopamine that is capable of converting exogenous energy into heat.

63. The theragnostic particle of claim 56, wherein the second material absorbs light at a wavelength ranging from 400 nm to 750 nm.

64. The theragnostic particle of claim 56, wherein the second material has significant absorption of photonic energy in the near infrared spectral region having a wavelength range from 750 nm to 1100 nm.

65. The theragnostic particle of any one of claim 63 or 64, wherein the second material is selected from the group consisting of a tetrakis aminium dye, a cyanine dye, a squarylium dye, indocyanine green (ICG), new ICG (IR 820), squaraine dye, IR 780 dye, IR 193 dye, Epolight™ 1117 dye, iron oxide thin layer coating, iron oxide, zinc iron phosphate pigment, and combinations thereof.

66. The theragnostic particle of any one of claims 56-65, wherein the carrier comprises a biocompatible substance selected from the group consisting of a lipid, polymer-lipid conjugate, carbohydrate-lipid conjugate, peptide-lipid conjugate, protein-lipid conjugate, an inorganic polymer, polyester, a polyester, a polyurea, a polyanhydride, a polysaccharide, a polyphosphoester, a poly(ortho ester), a poly(amino acid), a protein, dendritic polylysine, and combinations thereof.

67. A method for diagnosing and killing of drug resistant microbes at a site comprising:
administering an effective amount of the theragnostic particles of the claim 56 to the site;
contacting the theragnostic particles with a milieu near the site;
waiting for a period time to observe the presence or absence of optical response, and when an optical response is observed indicating the presence of the drug resistant microbes, then employing an exogenous energy source at the site;
wherein the theragnostic particles absorb energy from the exogenous energy source and converts the absorbed energy into heat;
wherein the heat travels outside the theragnostic particle to induce localized hyperthermia at a temperature ranging from about 38.0° C. to about 52.0° C. in an area surrounding the theragnostic particle;
wherein the localized hyperthermia lasts for a sufficient period of time to cause the death of the drug resistant microbes.

68. A diagnostic composition of claim 39 comprising a diagnostic particle of claim 51.

69. A diagnostic composition of claim 39 comprising a diagnostic particle of claim 56.

70. A method for detecting the presence or absence of a drug resistant microbes in a sample comprising the steps of:
(1) providing the diagnostic particles of claim 51;
(2) mixing the diagnostic particles with the sample;
(3) observing the absence or presence of an optical response in the sample;
wherein the presence of the optical response indicates the presence of the drug resistant microbes;
wherein the antimicrobial inactivating factor causes degradation of the FG to release the spectroscopic probe and result in a detectable optical response.

71. The method of claim 70, wherein the optical response is a color change within the visible region of the electromagnetic spectrum.

72. The method of claim 70, wherein the optical response is fluorescence.

73. The method of claim 70, further comprising a step of quantifying the optical response using spectroscopy for determining the bacterial burden to inform antibiotic selection and dosage thereof.

74. A kit for detecting the presence of drug resistant bacteria, comprising:
the biosensor of claim 1; and
an instruction sheet providing instructions to a human subject,
wherein the instructions comprise:
collect a sample;
contact the biosensor with the sample; and
observe the presence or absence of the optical response.

75. A kit for detecting and killing drug resistant bacteria, comprising:
a composition comprising the theragnostic particle of claim 56; and
an instruction sheet providing instructions to a human subject,
wherein the instructions comprise:
collect a sample;
contact the composition comprising the theragnostic particle with the sample;
observe the presence or absence of the optical response; and
upon observing an optical response, exposing the sample to the exogenous source.

76. A colorimetric biosensor, comprising: (1) a chromogenic probe or fluorogenic probe, (2) a bimodal sensing component for β-lactamase having a first material derived from β-lactam antibiotic and a third material derived from β-lactamase inhibitor, wherein
β-lactamase degrades the first material to release the chromogenic probe or fluorogenic probe to produce detectable optical response, and
wherein the third material in the biosensor acts to enhance the selectivity toward microbes that secrete specific type antibiotic inactivating factor.

77. The colorimetric biosensor of claim 75, wherein the optical response comprises a change of color or emission of fluorescence.

78. The colorimetric biosensor of any one of claims 75-76, wherein the third material is derived from one or more of a β-lactamase inhibitor.

79. The colorimetric biosensor of any one of claims 75-76, wherein the third material is derived from an ESBL inhibitor.

80. A biosensor for the detection of drug resistant bacteria comprising a Dithiofluorescein-Cephalosporin conjugate of Formula 12: [chemical structure]
